(19) United States
(12) Patent Application Publication
(10) Pub. No.: US 2025/0215077 A1
(43) Pub. Date: Jul. 3, 2025

(54) MONOVALENT ANTI-TREM2 BINDING MOLECULES AND METHODS OF USE THEREOF

(71) Applicant: DENALI THERAPEUTICS INC., South San Francisco, CA (US)

(72) Inventors: Mark S. DENNIS, San Carlos, CA (US); Gilbert DI PAOLO, San Francisco, CA (US); Anuja R. MODI, Burlingame, CA (US); Kathryn M. MONROE, San Francisco, CA (US); Adam P. SILVERMAN, San Carlos, CA (US); Bettina VAN LENGERICH, Burlingame, CA (US); Lihong ZHAN, Daly City, CA (US)

(73) Assignee: DENALI THERAPEUTICS INC., South San Francisco, CA (US)

(21) Appl. No.: 18/850,632

(22) PCT Filed: Mar. 28, 2023
(86) PCT No.: PCT/US2023/016578

§ 371 Date: Sep. 25, 2024

Related U.S. Application Data
(60) Provisional application No. 63/324,410, filed on Mar. 28, 2022.

(51) Int. Cl. C07K 16/28; A61K 39/00

(52) U.S. Cl. CPC C07K 16/2803; C07K 16/2881; A61K 2039/505; C07K 2317/31; C07K 2317/35; C07K 2317/52; C07K 2317/55; C07K 2317/77; C07K 2317/92

(57) ABSTRACT

In one aspect, monovalent binding molecules that specifically bind to a human triggering receptor expressed on myeloid cells 2 (TREM2) protein are provided, as well as methods of use thereof.

### CROSS-REFERENCE TO RELATED APPLICATION

This application claims priority to U.S. Provisional Application Ser. No. 63/324,410, filed Mar. 28, 2022. The entire content of the application referenced above is hereby incorporated by reference herein.

### BACKGROUND

Triggering receptor expressed on myeloid cells-2 (TREM2) is a transmembrane receptor that is expressed on microglia and is believed to function in regulating phagocytosis, cell survival, and the production of pro-inflammatory cytokines. Mutations in TREM2 have been identified in neurodegenerative diseases including Alzheimer's disease, Nasu-Hakola disease, Parkinson's disease, amyotrophic lateral sclerosis, and frontotemporal dementia. While various antibodies targeting TREM2 have been developed, anti-TREM2 activating antibodies have been reported to require bivalent binding (Schlepckow et al., EMBO Mol Med. (2020) 12(4):e11227; Ellwanger et al., Proc Natl Acad Sci USA. (2021) 118(3):e2017742118). Binding molecules with alternative configurations, such as those having monovalent binding capabilities, are a valuable resource for the development of new therapeutics. For example, monovalent binding proteins may be used to generate bispecific antibodies (bsAbs), which have the capability of recognizing two different epitopes.

Accordingly, there is a need for new monovalent anti-TREM2 binding molecules, as well as new approaches for designing such molecules.

### BRIEF SUMMARY

Certain embodiments provide a molecule comprising:
(a) a first binding region that specifically binds to TREM2; and
(b) a second binding region that specifically binds to a transferrin receptor;
wherein the molecule is monovalent for binding to TREM2.

Certain embodiments provide a pharmaceutical composition comprising a molecule as described herein and a pharmaceutically acceptable carrier.

Certain embodiments provide a method of treating a neurodegenerative disease in a subject, comprising administering to the subject a molecule as described herein or a pharmaceutical composition as described herein.

Certain embodiments provide a molecule as described herein or a pharmaceutical composition as described herein for use in treating a neurodegenerative disease in a subject.

Certain embodiments provide the use of a molecule as described herein in the preparation of a medicament for treating a neurodegenerative disease in a subject.

Certain embodiments provide a method of enhancing TREM2 activity in a subject having a neurodegenerative disease, comprising administering to the subject a molecule as described herein or a pharmaceutical composition as described herein.

Certain embodiments provide a molecule as described herein or a pharmaceutical composition as described herein for use in enhancing TREM2 activity in a subject having a neurodegenerative disease.

Certain embodiments provide the use of a molecule as described herein in the preparation of a medicament for enhancing TREM2 activity in a subject having a neurodegenerative disease.

Certain embodiments provide an isolated polynucleotide comprising a nucleotide sequence encoding a molecule as described herein.

Certain embodiments provide an isolated polynucleotide comprising a nucleotide sequence encoding a light chain, a heavy chain and/or a Fc polypeptide as described herein. Certain embodiments provide a plurality of polynucleotides, wherein the plurality comprises a polynucleotide comprising a nucleotide sequence encoding a light chain as described herein, a polynucleotide comprising a nucleotide sequence encoding an Fc polypeptide as described herein, and a polynucleotide comprising a nucleotide sequence encoding a heavy chain (e.g., a heavy chain comprising a Fc polypeptide capable of binding to a TfR; or a heavy chain linked to a second binding region) as described herein.

Certain embodiments provide a vector comprising the polynucleotide as described herein or a plurality of polynucleotides as described herein.

Certain embodiments provide one or more vectors comprising a plurality of polynucleotides as described herein.

Certain embodiments provide a host cell comprising the polynucleotide or plurality of polynucleotides as described herein or a vector(s) as described herein.

Certain embodiments provide a method of expressing a molecule that specifically binds to TREM2, comprising: culturing a host cell as described herein under conditions suitable for expression of the molecule.

Certain embodiments provide a method of enhancing the activity of a monovalent anti-TREM2 molecule, the method comprising modifying the molecule to specifically bind to a transferrin receptor.

### BRIEF DESCRIPTION OF THE DRAWINGS

FIGS. 1A-1B. FIG. 1A. Illustration of an exemplary monovalent anti-TREM2 binding molecule comprising a single Fab fragment and a Fc polypeptide that has been modified to specifically bind to a transferrin receptor. FIG. 1B. Illustration of an exemplary monovalent anti-TREM2 binding molecule, which comprises an Fc polypeptide dimer, wherein an anti-TREM2 Fab fragment is linked to the N-terminus of one of the Fc polypeptides and a single chain Fab (scFab) that specifically binds to a transferrin receptor linked to the C-terminus of the Fc polypeptide.

FIGS. 2A-2G. Monovalent ATV:TREM2 (ATV:TREM2 MV), which comprises a single Fab, 1) potentiates TREM2 receptor signaling; and 2) promotes TfR-TREM2 receptor interactions which drives endocytosis and internalization in a synergistic manner. (FIG. 2A) Schematic illustration of the impact of ATV and TREM2 Fab valency antibodies on pSyk signaling activity. Antibody versions include anti-TREM2; anti-TREM2 with a mono-Fab (MV); and ATV:TREM2 with a mono-Fab (MV). (FIG. 2B) pSyk detected by AlphaLISA in hTREM2-DAP12 HEK293 cells treated with a dose response of anti-TREM2, anti-TREM2 MV, and ATV:TREM2 MV for 5 min at 37° C. Data represents mean+/−SEM from n=3 independent experiments each normalized to vehicle control (PBS). (FIG. 2C) pSyk activation by ATV: TREM2 MV is blocked by co-treatment with anti-TfR. hTREM2-DAP12 HEK293 cells were co-dosed with 100 nM anti-TREM2, or anti-TREM2 MV, or ATV:TREM2 MV and a titration of blocking anti-TfR antibody that competes for the ATV binding site for 5 min at 37° C. pSyk was measured by AlphaLisa. Data normalized to vehicle control (PBS) (n=3 independent experiments). (FIG. 2D) Quantification of spot intensity for IgG and pSyk immunofluorescence per cell (n=3 independent experiments, data is represented as mean+/−SEM, unpaired t-test (*:p<0.05; **:p<0.01; ***:p<0.001; ****: p<0.0001). (FIGS. 2E, 2F) Quantification of percent of IgG or pSyk spots localized within EEA1-positive (FIG. 2E) or Tf-positive (FIG. 2F) endosomes. (Data is represented as n=3 independent experiments with each 3000-5000 cells per condition, mean+/−SEM, unpaired t-test (*:p<0.05; **:p<0.01; ***:p<0.001; ****:p<0.0001). (FIG. 2G) Surface and total TREM2 receptor levels measured by immunofluorescence microscopy of hTREM2-DAP12 HEK293 cells demonstrates a trend toward an increased reduction of surface TREM2 levels with ATV:TREM2 MV vs anti-TREM2 MV, yet no changes in total TREM2 levels, consistent with re-distribution of the receptor from the plasma membrane to endosomes. Data is represented as mean+/−SEM of 3-4 independent experiments, unpaired t-test p-value ***: p<0.001; ****: p<0.0001.

FIGS. 3A-3B. hTREM2 pSYK Signaling. FIG. 3A. hTREM2 pSYK signaling evaluated with bivalent (BV) molecules: anti-TREM2 BV linked to an anti-TfR scFab; and anti-TREM2 BV (n=3 bio replicates; mean±SEM). FIG. 3B. hTREM2 pSYK signaling evaluated with monovalent (MV) molecules: anti-TREM2 MV linked to an anti-TfR scFab; and ATV:TREM2 MV (n=3 bio replicates; mean±SEM).

FIG. 4. Ratio of bound and total sTREM2 in brain lysates. Bound and total sTREM2 levels were measured in the brain lysates 24 hours and 96 hours post dose and the ratio was calculated to indicate the target engagement of TREM2 antibodies in the brain. Data is represented as mean+/−SEM.

FIGS. 5A-5B. Effect of ATV:TREM2 MV on CSF1R in brain. CSF1R levels were measured in brains 24 hours (FIG. 5A) or 96 hours (FIG. 5B) post single dose. Data is represented as mean+/−SEM, unpaired t-test p-value*: p<0.05.

FIGS. 6A-6D. Effect of ATV:TREM2 MV on IP-10 and MCP-5 in brain. (FIG. 6A, 6B) Quantification of IP-10 in brains collected 24 hours or 96 hours post single dose. (FIG. 6C, 6D) Quantification of MCP-5 in brains collected 24 hours or 96 hours post single dose. Data is represented as mean+/−SEM, unpaired t-test p-value***: p<0.001; ****: p<0.0001.

### DETAILED DESCRIPTION

### I. Introduction

TREM2 is a transmembrane receptor that is expressed on the cell surface of microglia, dendritic cells, macrophages, and osteoclasts. Without being bound to a particular theory, it is believed that upon ligand binding, TREM2 forms a signaling complex with a transmembrane adapter protein, DNAX-activating protein 12 (DAP12), which in turn is tyrosine phosphorylated by the protein kinase SRC. It is believed that the activated TREM2/DAP12 signaling complex mediates intracellular signaling by recruiting and phosphorylating kinases such as Syk kinase. TREM2/DAP12 signaling modulates activities such as phagocytosis, cell growth and survival, pro-inflammatory cytokine secretion, and the migration of cells such as microglia and macrophages. TREM2 undergoes regulated intramembrane proteolysis, in which the membrane-associated full-length TREM2 is cleaved by the metalloprotease ADAM10 into a sTREM2 portion that is shed from the cell and a membrane-retained C-terminal fragment that is further degraded by a gamma-secretase. Altered levels of sTREM2 have been reported in patients having Alzheimer's disease or frontotemporal dementia and having a mutation in TREM2. Additionally, mutations in TREM2 are associated with altered functions such as impaired phagocytosis and reduced microglial function.

As detailed in the Examples section below, monovalent anti-TREM2 binding molecules, which comprise a second binding region that specifically binds to a transferrin receptor (TfR), were generated. As discussed in Example 2, a monovalent anti-TREM2 antibody (ATV:TREM2 MV) was capable of engaging both TREM2 and TfR, which resulted in enhanced TREM2 signaling at the cellular level. These results were surprising and in contrast to a monovalent anti-TREM2 antibody that had not been engineered to bind to TfR, which showed no activity.

### II. Definitions

As used herein, the singular forms “a” “an” and “the” include plural referents unless the content clearly dictates otherwise. Thus, for example, reference to “an antibody” optionally includes a combination of two or more such molecules, and the like.

As used herein, the terms “about” and “approximately,” when used to modify an amount specified in a numeric value or range, indicate that the numeric value as well as reasonable deviations from the value known to the skilled person in the art, for example±20%, ±10%, or 5%, are within the intended meaning of the recited value.

As used herein, the term “TREM2 protein” refers to a triggering receptor expressed on myeloid cells 2 protein that is encoded by the gene TREM2. As used herein, a “TREM2 protein” refers to a native (i.e., wild-type) TREM2 protein of any vertebrate, such as but not limited to humans, non-human primates (e.g., cynomolgus monkey), rodents (e.g., mice, rat), and other mammals. In some embodiments, a TREM2 protein is a human TREM2 protein having the sequence identified in UniprotKB accession number Q9NZC2 (SEQ ID NO:1).

As used herein, the term “antibody” refers to a protein with an immunoglobulin fold that specifically binds to an antigen via its variable regions. The term encompasses intact polyclonal antibodies, intact monoclonal antibodies, single chain antibodies, multispecific antibodies such as bispecific antibodies, monospecific antibodies, monovalent antibodies, chimeric antibodies, humanized antibodies, and human antibodies. The term “antibody,” as used herein, also includes antibody fragments that retain binding specificity via its variable regions, including but not limited to Fab, F(ab′)₂, Fv, scFv, and bivalent scFv. Antibodies can contain light chains that are classified as either kappa or lambda. Antibodies can contain heavy chains that are classified as gamma, mu, alpha, delta, or epsilon, which in turn define the immunoglobulin classes, IgG, IgM, IgA, IgD and IgE, respectively.

As used herein, the term “anti-TREM2 antibody” refers to an antibody that specifically binds to a TREM2 protein (e.g., human TREM2).

As used herein, the term “monovalent” refers to a molecule having a single binding region having affinity for a given epitope or antigen. Thus, a “monovalent anti-TREM2 molecule” would comprise a single binding region having affinity for TREM2. Similarly, a “monovalent anti-TfR molecule” would comprise a single binding region having affinity for TfR.

As used herein, the term “monovalent antibody” refers to an antibody that includes a binding region (e.g., a single Fv or Fab fragment) having affinity for a given epitope or antigen. The monovalent antibody may comprise only a single binding region or may comprise additional binding regions that recognize other epitopes or antigens. Thus, the term “monovalent anti-TREM2 antibody” refers to an antibody that includes a single binding region (e.g., a single Fv or Fab fragment) having affinity for TREM2.

The term “binding region” refers to a portion of a molecule that binds to an epitope or target antigen. For example, the binding region may comprise an amino acid sequence, such as an antibody variable region sequence, that binds to a target epitope or antigen. Thus, in certain embodiments, the binding region is an antigen binding fragment or antigen binding portion of an antibody.

As used herein, the term “antigen binding portion” refers to an antigen binding segment or entity that specifically binds to a given antigen or protein. The terms “antigen-binding portion” and “antigen-binding fragment” are used interchangeably herein and refer to one or more fragments of an antibody that retains the ability to specifically bind to an antigen (e.g., a TREM2 protein) via its variable region. Examples of antigen-binding fragments include, but are not limited to, a Fab fragment (a monovalent fragment consisting of the V_{L}, V_{H}, CL and CH1 domains along with at least a partial hinge sequence), F(ab′)₂ fragment (a bivalent fragment comprising two Fab fragments linked by a disulfide bridge at the hinge region), single chain Fv (scFv), disulfide-linked Fv (dsFv), complementarity determining regions (CDRs), a V_{L} (light chain variable region), and a V_{H} (heavy chain variable region).

As used herein, the term “anti-TREM2 antigen binding portion” refers to an antigen binding segment or entity that specifically binds to a TREM2 protein (e.g., human TREM2).

As used herein, the term “anti-TfR antigen binding portion” refers to an antigen binding segment or entity that specifically binds to a TfR protein (e.g., human TfR).

The term “variable region” or “variable domain” refers to a domain in an antibody heavy chain or light chain that is derived from a germline Variable (V) gene, Diversity (D) gene, or Joining (J) gene (and not derived from a Constant (Cμ and Cδ) gene segment), and that gives an antibody its specificity for binding to an antigen. Typically, an antibody variable region comprises four conserved “framework” regions interspersed with three hypervariable “complementarity determining regions.”

The term “complementarity determining region” or “CDR” refers to the three hypervariable regions in each chain that interrupt the four framework regions established by the light and heavy chain variable regions. The CDRs are primarily responsible for antibody binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 or CDR-H3 is located in the variable region of the heavy chain of the antibody in which it is found, whereas a V_{L} CDR1 or CDR-L1 is the CDR1 from the variable region of the light chain of the antibody in which it is found.

The “framework regions” or “FRs” of different light or heavy chains are relatively conserved within a species. The framework region of an antibody, that is the combined framework regions of the constituent light and heavy chains, serves to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

The amino acid sequences of the CDRs and framework regions can be determined using various well-known definitions in the art, e.g., Kabat, Chothia, international ImMunoGeneTics database (IMGT), AbM, and observed antigen contacts (“Contact”). In some embodiments, CDRs are determined according to the Contact definition. See, MacCallum et al., J. Mol. Biol., 262:732-745 (1996). In some embodiments, CDRs are determined by a combination of Kabat, Chothia, and/or Contact CDR definitions.

The term “epitope” refers to the area or region of an antigen to which the CDRs of an antibody specifically binds and can include a few amino acids or portions of a few amino acids, e.g., 5 or 6, or more, e.g., 20 or more amino acids, or portions of those amino acids. For example, where the target is a protein, the epitope can be comprised of consecutive amino acids (e.g., a linear epitope), or amino acids from different parts of the protein that are brought into proximity by protein folding (e.g., a discontinuous or conformational epitope). In some embodiments, the epitope is phosphorylated at one amino acid (e.g., at a serine or threonine residue).

As used herein, the phrase “recognizes an epitope,” as used with reference to an anti-TREM2 antibody, means that the antibody CDRs interact with or specifically bind to the antigen (i.e., the TREM2 protein) at that epitope or a portion of the antigen containing that epitope.

A “monoclonal antibody” refers to antibodies produced by a single clone of cells or a single cell line and consisting of or consisting essentially of antibody molecules that are identical in their primary amino acid sequence.

A “polyclonal antibody” refers to an antibody obtained from a heterogeneous population of antibodies in which different antibodies in the population bind to different epitopes of an antigen.

A “chimeric antibody” refers to an antibody molecule in which the constant region, or a portion thereof, is altered, replaced or exchanged so that the antigen-binding site (i.e., variable region, CDR, or portion thereof) is linked to a constant region of a different or altered class, effector function and/or species, or in which the variable region, or a portion thereof, is altered, replaced or exchanged with a variable region having a different or altered antigen specificity (e.g., CDR and framework regions from different species). In some embodiments, a chimeric antibody is a monoclonal antibody comprising a variable region from one source or species (e.g., mouse) and a constant region derived from a second source or species (e.g., human). Methods for producing chimeric antibodies are described in the art.

A “humanized antibody” is a chimeric immunoglobulin derived from a non-human source (e.g., murine) that contains minimal sequences derived from the non-human immunoglobulin outside the CDRs. In general, a humanized antibody will comprise at least one (e.g., two) antigen-binding variable domain(s), in which the CDR regions substantially correspond to those of the non-human immunoglobulin and the framework regions substantially correspond to those of a human immunoglobulin sequence. The humanized antibody can also comprise at least a portion of an immunoglobulin constant region (Fc), typically that of a human immunoglobulin sequence. Methods of antibody humanization are known in the art.

A “human antibody” or a “fully human antibody” is an antibody having human heavy chain and light chain sequences, typically derived from human germline genes. In some embodiments, the antibody is produced by a human cell, by a non-human animal that utilizes human antibody repertoires (e.g., transgenic mice that are genetically engineered to express human antibody sequences), or by phage display platforms.

The term “specifically binds” refers to a molecule (e.g., an antibody or an antigen-binding portion thereof) that binds to an epitope or target with greater affinity, greater avidity, and/or greater duration to that epitope or target in a sample than it binds to another epitope or non-target compound (e.g., a structurally different antigen). In some embodiments, an antibody (or an antigen-binding portion thereof) that specifically binds to an epitope or target is an antibody (or an antigen-binding portion thereof) that binds to the epitope or target with at least 5-fold greater affinity than other epitopes or non-target compounds, e.g., at least 5-fold, 10-fold, 100-fold, 1,000-fold, 10,000-fold, or greater affinity. The term “specific binding,” “specifically binds to,” or “is specific for” a particular epitope or target, as used herein, can be exhibited, for example, by a molecule having an equilibrium dissociation constant K_{D} for the epitope or target to which it binds of, e.g., 10⁻⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. It will be recognized by one of skill that an antibody that specifically binds to a target (e.g., a TREM2 protein) from one species may also specifically bind to orthologs of that target (e.g., the TREM2 protein).

The term “binding affinity” is used herein to refer to the strength of a non-covalent interaction between two molecules, e.g., between an antibody (or an antigen-binding portion thereof) and an antigen. Thus, for example, the term may refer to 1:1 interactions between an antibody (or an antigen-binding portion thereof) and an antigen, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet platform). As used herein, “binding affinity” includes not only formal binding affinities, such as those reflecting 1:1 interactions between an antibody (or an antigen-binding portion thereof) and an antigen, but also apparent affinities for which K_{D} values are calculated that may reflect avid binding.

The term “cross-reacts,” as used herein, refers to the ability of an antibody to bind to an antigen other than the antigen against which the antibody was raised. In some embodiments, cross-reactivity refers to the ability of an antibody to bind to an antigen from another species than the antigen against which the antibody was raised. As a non-limiting example, an anti-TREM2 antibody as described herein that is raised against a human TREM2 peptide can exhibit cross-reactivity with a TREM2 peptide or protein from a different species (e.g., monkey or mouse).

A “transferrin receptor” or “TfR” as used herein refers to transferrin receptor protein 1. The human transferrin receptor 1 polypeptide sequence is set forth in SEQ ID NO:62. Transferrin receptor protein 1 sequences from other species are also known (e.g., chimpanzee, accession number XP_003310238.1; rhesus monkey, NP_001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full length transferrin receptor protein includes a short N-terminal intracellular region, a transmembrane region, and a large extracellular domain. The extracellular domain is characterized by three domains: a protease-like domain, a helical domain, and an apical domain. The apical domain sequence of human transferrin receptor 1 is set forth in SEQ ID NO:55.

The terms “CH3 domain” and “CH2 domain” as used herein refer to immunoglobulin constant region domain polypeptides. In the context of IgG antibodies, a CH3 domain polypeptide refers to the segment of amino acids from about position 341 to about position 447 as numbered according to the EU numbering scheme, and a CH2 domain polypeptide refers to the segment of amino acids from about position 231 to about position 340 as numbered according to the EU numbering scheme and does not include hinge region sequences. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. In the context of IgG antibodies, an Fc region refers to the segment of amino acids from about position 231 to about position 447 as numbered according to the EU numbering scheme. As used herein, the term “Fc region” may also include at least a part of a hinge region of an antibody. An illustrative hinge region sequence is the human IgG1 hinge sequence EPKSCDKTHTCPPCP (SEQ ID NO:56) and an exemplary partial hinge region sequence is set forth in SEQ ID NO:57.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a polypeptide “corresponds to” an amino acid in the region of SEQ ID NO:38 from amino acids 111-217 when the residue aligns with the amino acid in SEQ ID NO:38 when optimally aligned to SEQ ID NO:38. The polypeptide that is aligned to the reference sequence need not be the same length as the reference sequence.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region, but does not contain a variable region.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

The term “isolated,” as used with reference to a nucleic acid or protein (e.g., antibody), denotes that the nucleic acid or protein is essentially free of other cellular components with which it is associated in the natural state. Purity and homogeneity are typically determined using analytical chemistry techniques such as electrophoresis (e.g., polyacrylamide gel electrophoresis) or chromatography (e.g., high performance liquid chromatography). In some embodiments, an isolated nucleic acid or protein (e.g., antibody) is at least 85% pure, at least 90% pure, at least 95% pure, or at least 99% pure.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof. “Amino acid analogs” refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions.

The terms “polynucleotide” and “nucleic acid” interchangeably refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

The terms “conservative substitution” and “conservative mutation” refer to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60%, at least 65%, at least 70%, at least 75%, at least 80%, at least 85%, at least 90%, or at least 95% or greater, that are identical over a specified region when compared and aligned for maximum correspondence over a comparison window, or designated region as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the subject, individual, or patient is a human.

The terms “treating,” “treatment,” and the like are used herein to generally mean obtaining a desired pharmacologic and/or physiologic effect. “Treating” or “treatment” may refer to any indicia of success in the treatment or amelioration of a neurodegenerative disease (e.g., Alzheimer's disease or another neurodegenerative disease described herein), including any objective or subjective parameter such as abatement, remission, improvement in patient survival, increase in survival time or rate, diminishing of symptoms or making the disease more tolerable to the patient, slowing in the rate of degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

The term “pharmaceutically acceptable excipient” refers to a non-active pharmaceutical ingredient that is biologically or pharmacologically compatible for use in humans or animals, such as, but not limited to a buffer, carrier, or preservative.

As used herein, a “therapeutic amount” or “therapeutically effective amount” of an agent (e.g., an antibody as described herein) is an amount of the agent that treats, alleviates, abates, or reduces the severity of symptoms of a disease in a subject. A “therapeutic amount” of an agent (e.g., an antibody as described herein) may improve patient survival, increase survival time or rate, diminish symptoms, make an injury, disease, or condition (e.g., a neurodegenerative disease) more tolerable, slow the rate of degeneration or decline, or improve a patient's physical or mental well-being.

The term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, an antibody as described herein is administered intravenously.

The term “control” or “control value” refers to a reference value or baseline value. Appropriate controls can be determined by one skilled in the art. In some instances, control values can be determined relative to a baseline within the same subject or experiment, e.g., a measurement of sTREM2 taken prior to treatment with a monovalent anti-TREM2 molecule that binds a TfR can be a control value for a post-treatment measurement of sTREM2 levels in the same subject. In other instances, the control value can be determined relative to a control subject (e.g., a healthy control or a disease control) or an average value in a population of control subjects (e.g., healthy controls or disease controls, e.g., a population of 10, 20, 50, 100, 200, 500, 1000 control subjects or more), e.g., a measurement of a subject's level of sTREM2 either at baseline or after treatment can be compared to a healthy control value.

### III. Monovalent Anti-TREM2 Binding Molecules that Also Bind a TfR

As described herein, certain monovalent anti-TREM2 binding molecules are provided, which have, e.g., an enhanced ability to modulate (e.g., increase) the activity of TREM2. In particular, certain embodiments provide a molecule comprising:
(a) a first binding region that specifically binds to TREM2; and
(b) a second binding region that specifically binds to a transferrin receptor (TfR);
wherein the molecule is monovalent for binding to TREM2.

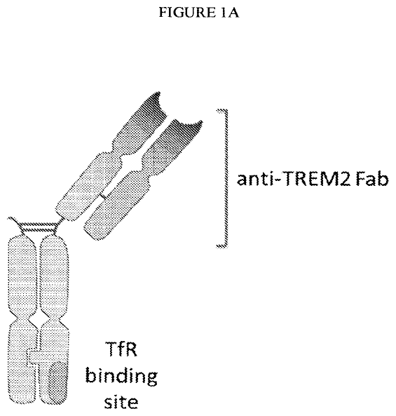

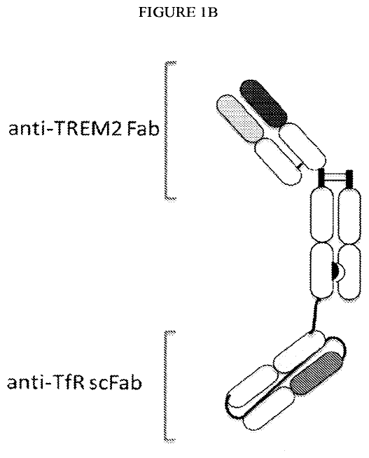

In some embodiments, the molecule is an antibody or comprises an antibody (e.g., an antibody having a traditional structure, an engineered antibody having a modified structure, or an antibody fragment (see, e.g., FIGS. 1A-1)). In certain embodiments, the molecule is or comprises a chimeric antibody. In some embodiments, the molecule is or comprises a humanized and/or affinity matured antibody.

For example, in certain embodiments, the molecule comprises an antibody variable region sequence(s). In particular, in certain embodiments, the first binding region that specifically binds to TREM2 and/or second binding region that specifically binds to a TfR comprises an antibody variable region sequence(s).

In certain embodiments, the first binding region and/or second binding region comprises or consists of a Fv region. In certain embodiments, the first binding region and/or second binding region is a single chain Fv (scFV) or a disulfide-stabilized Fv (dsFv).

In certain embodiments, the first binding region and/or second binding region comprises or consists of a Fab fragment. In certain embodiments, the first binding region and/or second binding region is a Fab fragment. In certain embodiments, the first binding region is a Fab fragment. In certain embodiments, the second binding region is a Fab fragment. In certain embodiments, the first binding region and/or second binding region is a single chain Fab fragment (scFab). In certain embodiments, the first binding region is a scFab. In certain embodiments, the second binding region is a scFab.

In certain embodiments, a molecule as described herein further comprises a first Fc polypeptide. In certain embodiments, a molecule as described herein further comprises a second Fc polypeptide, wherein the second Fc polypeptide forms an Fc dimer with the first Fc polypeptide.

In some embodiments, one or both of the Fc polypeptides may further comprise a partial or full hinge region at the N-terminal region of the Fc polypeptide. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:56) or a portion thereof (e.g., DKTHTCPPCP; SEQ ID NO:57). In certain embodiments, a binding region as described herein is linked to the N-terminus of a Fc polypeptide through a hinge region. In other embodiments, the N-terminus of an Fc polypeptide is not linked to a binding region but comprises a partial or full hinge region (e.g., a partial hinge region). For example, in certain embodiments, a binding region as described herein may be linked to the N-terminus of an Fc polypeptide such that a resulting heavy chain comprises a full hinge region; and the N-terminus of the other Fc polypeptide includes a portion of a hinge region (e.g., a portion of an IgG1 hinge region, such as DKTHTCPPCP (SEQ ID NO:57). In other embodiments, both Fc polypeptides are comprised within heavy chains that comprise a full hinge region.

In certain embodiments, the first binding region is linked to the first or the second Fc polypeptide. In certain embodiments, the first binding region is linked to the N-terminus of the first or the second Fc polypeptide. In certain embodiments, the first binding region is linked to the N-terminus of the first Fc polypeptide. In certain embodiments, the first binding region is linked to the N-terminus of the second Fc polypeptide. In certain embodiments, the first binding region is linked to the C-terminus of the first or the second Fc polypeptide. In certain embodiments, the first binding region is linked to the C-terminus of the first Fc polypeptide. In certain embodiments, the first binding region is linked to the C-terminus of the second Fc polypeptide.

In certain embodiments, the second binding region is linked to the first or the second Fc polypeptide. In certain embodiments, the second binding region is linked to the N-terminus of the first or the second Fc polypeptide. in certain embodiments, the second binding region is linked to the N-terminus of the first Fc polypeptide. In certain embodiments, the second binding region is linked to the N-terminus of the second Fc polypeptide. In certain embodiments, the second binding region is linked to the C-terminus of the first or the second Fc polypeptide. In certain embodiments, the second binding region is linked to the C-terminus of the first Fc polypeptide. In certain embodiments, the second binding region is linked to the C-terminus of the second Fc polypeptide.

In certain embodiments, the first binding region is linked to the N-terminus of the first Fc polypeptide and the second binding region is linked to the N-terminus of the second Fc polypeptide.

In certain embodiments, the first binding region is linked to the N-terminus of the first Fc polypeptide and the second binding region is linked to the C-terminus of the first Fc polypeptide. In certain embodiments, the first binding region is linked to the N-terminus of the first Fc polypeptide and the second binding region is linked to the C-terminus of the second Fc polypeptide.

In some embodiments, the second binding region is linked to an Fc polypeptide (e.g., to the C-terminus of a Fc polypeptide, such as the C-terminus of the first Fc polypeptide) by a linker, e.g., a peptide linker. In some embodiments, the Fc polypeptide is joined to the second binding region by a peptide bond or by a peptide linker, e.g., is a fusion polypeptide. The peptide linker may be configured such that it allows for the rotation of the second binding region relative to the Fc polypeptide to which it is joined; and/or is resistant to digestion by proteases. Peptide linkers may contain natural amino acids, unnatural amino acids, or a combination thereof. In some embodiments, the peptide linker may be a flexible linker, e.g., containing amino acids such as Gly, Asn, Ser, Thr, Ala, and the like (e.g., a glycine-rich linker). Such linkers are designed using known parameters and may be of any length and contain any number of repeat units of any length (e.g., repeat units of Gly and Ser residues). For example, the linker may have repeats, such as two, three, four, five, or more Gly₄-Ser (SEQ ID NO:172) repeats (see, e.g., SEQ ID NO:173) or a single Gly₄-Ser (SEQ ID NO: 172). In certain aspects, the linker may be GGSGGGGSGGGGSGGGGS (SEQ ID NO:148). In other embodiments, the second binding region is joined to the Fc polypeptide by a chemical cross-linking agent.

In certain other embodiments, the second binding region is comprised within the first or the second Fc polypeptide. In particular, as described herein, a Fc polypeptide may be modified to specifically bind to a TfR. In certain embodiments, the first binding region is linked to the first or second Fc polypeptide and the second binding region is comprised within the first or the second Fc polypeptide. In certain embodiments, the first binding region is linked to the N-terminus of the first or second Fc polypeptide and the second binding region is comprised within the first or the second Fc polypeptide. In certain embodiments, the first binding region is linked to the N-terminus of the first Fc polypeptide and the second binding region is comprised within the first or the second Fc polypeptide. In certain embodiments, the first binding region is linked to the N-terminus of the first Fc polypeptide and the second binding region is comprised within the first Fc polypeptide. In certain embodiments, the first binding region is linked to the N-terminus of the first Fc polypeptide and the second binding region is comprised within the second Fc polypeptide.

In certain embodiments, a molecule as described herein may further comprise a third binding region that specifically binds to a target protein of interest (e.g., the molecule could specifically bind to TREM2, TfR, and a third protein of interest). In certain embodiments, the third binding region comprises an antibody variable region sequence(s). In certain embodiments, the third binding region comprises or consists of a Fv region. In certain embodiments, the third binding region is a scFV or a dsFv. In certain embodiments, the third binding region comprises or consists of a Fab fragment. In certain embodiments, the third binding region is a Fab fragment. In certain embodiments, the third binding region is a scFab. In certain embodiments, the third binding region is linked to the first or the second Fc polypeptide. In certain embodiments, the third binding region is linked to the N-terminus of the first or the second Fc polypeptide. In certain embodiments, the third binding region is linked to the N-terminus of the first Fc polypeptide. In certain embodiments, the third binding region is linked to the N-terminus of the second Fc polypeptide. In certain embodiments, the third binding region is linked to the C-terminus of the first or the second Fc polypeptide. In certain embodiments, the third binding region is linked to the C-terminus of the first Fc polypeptide. In certain embodiments, the third binding region is linked to the C-terminus of the second Fc polypeptide.

In certain other embodiments, a molecule as described herein does not comprise a third binding region. For example, in certain embodiments, the molecule does not comprise a variable region sequence linked to the N-terminus of one of the Fc polypeptides present in the molecule (e.g., the N-terminus of the second Fc polypeptide is not linked to a variable region sequence).

In certain embodiments, a molecule as described herein comprises a first binding region as described herein, which is linked to the N-terminus of a first Fc polypeptide, a second binding region as described herein, which is linked to the C-terminus of the first Fc polypeptide (e.g., through a polypeptide linker), and a second Fc polypeptide that forms a dimer with the first polypeptide. In certain embodiments, the molecule further comprises a third binding region as described herein, which is linked to the N-terminus of the second Fc polypeptide. In certain other embodiments, the molecule does not comprise a third binding region. For example, in certain embodiments, the N-terminus of the second Fc polypeptide is not linked to a binding region but comprises a partial or full hinge region (e.g., a partial hinge region).

In certain embodiments, a molecule as described herein comprises a first binding region as described herein, which is linked to the N-terminus of a first Fc polypeptide, a second binding region as described herein, which is comprised within the first Fc polypeptide (e.g., the first Fc polypeptide is modified to bind to TfR), and a second Fc polypeptide that forms a dimer with the first polypeptide. In certain embodiments, the molecule further comprises a third binding region as described herein, which is linked to the N-terminus of the second Fc polypeptide. In certain other embodiments, the molecule does not comprise a third binding region. For example, in certain embodiments, the N-terminus of the second Fc polypeptide is not linked to a binding region but comprises a partial or full hinge region (e.g., a partial hinge region).

First Binding Region that Specifically Binds TREM2

Certain embodiments provide a monovalent anti-TREM2 molecule that binds TfR, which comprises a first binding region that specifically binds to TREM2. In some embodiments, the monovalent molecule is selective for TREM2 over other TREM-like receptors (e.g., TREM1).

In certain embodiments, the molecule is an agonist TREM2 binding molecule (i.e., it is capable activating TREM2 or increasing at least one biological activity of TREM2). In certain other embodiments, the molecule is an antagonist TREM2 binding molecule (i.e., it is capable inhibiting TREM2 or decreasing at least one biological activity of TREM2).

In certain embodiments, the first binding region comprises an antibody variable region sequence(s). Anti-TREM2 antibodies are known in the art and variable region sequences from such antibodies may be included in a first binding region (i.e., for specific binding to TREM2). For example, anti-TREM2 antibodies are described in, e.g., U.S. Pat. No. 11,186,636 and PCT Publication Nos. WO2021/113655, WO2021/146256, WO2020/172457, WO2020/172450, WO2019/055841, WO2019/118513, WO2019/028292, WO2018/195506, WO2017/062672, WO2017/058866, and WO2016/023019. As described herein, the activity of corresponding monovalent formats of these antibodies may be enhanced by the addition of a second binding region that specifically binds to a TfR. For example, in certain embodiments, variable region sequences from these antibodies may be incorporated into a monovalent anti-TREM2 binding molecule that comprises a second binding region as described herein. Certain examples of monovalent anti-TREM2 binding molecules and their associated sequences are described below and in Table 8.

For example, in some embodiments, a monovalent anti-TREM2 binding molecule comprises one or more complementarity determining region (CDR) sequences, a heavy chain variable region sequence, and/or a light chain variable region sequence as disclosed herein. In some embodiments, the molecule comprises one or more CDR sequences, a heavy chain variable region sequence, and/or a light chain variable region sequence as disclosed herein and further comprises one or more functional characteristics as disclosed herein, e.g., a molecule that enhances TREM2 activity.

In some embodiments, a heavy chain sequence, or a portion thereof, and/or a light chain sequence, or a portion thereof, is derived from an anti-TREM2 antibody described herein (e.g., Clone CL0020306, Clone CL0020188, Clone CL0020307, Clone A, Clone B, Clone C or Clone D). The CDR, heavy chain variable region, and light chain variable region amino acid sequences of these clones are set forth in Table 8.

### Clones CL0020188, CL0020306, CL0020307, and Variants of CL0020188

In some embodiments, the first binding region that specifically binds to TREM2 comprises one or more CDRs selected from the group consisting of:
(a) a heavy chain CDR1 (CDR-H1) comprising a sequence that has at least 90% sequence identity to the amino acid sequence of any one of SEQ ID NOS:4 and 12, or that has up to two amino acid substitutions relative to the amino acid sequence of any one of SEQ ID NOS:4 and 12;
(b) a heavy chain CDR2 (CDR-H2) comprising a sequence that has at least 90% sequence identity to the amino acid sequence of any one of SEQ ID NOS:5, 13, and 25, or that has up to two amino acid substitutions relative to the amino acid sequence of any one of SEQ ID NOS:5, 13, and 25;
(c) a heavy chain CDR3 (CDR-H3) comprising a sequence that has at least 90% sequence identity to the amino acid sequence of any one of SEQ ID NOS:6, 14, and 17, or that has up to two amino acid substitutions relative to the amino acid sequence of any one of SEQ ID NOS:6, 14, and 17;
(d) a light chain CDR1 (CDR-L1) comprising a sequence that has at least 90% sequence identity to the amino acid sequence of any one of SEQ ID NOS:7 and 23, or that has up to two amino acid substitutions relative to the amino acid sequence of any one of SEQ ID NOS:7 and 23;
(e) a light chain CDR2 (CDR-L2) comprising a sequence that has at least 90% sequence identity to the amino acid sequence of SEQ ID NO:8, or that has up to two amino acid substitutions relative to the amino acid sequence of SEQ ID NO:8; and
(f) a light chain CDR3 (CDR-L3) comprising a sequence that has at least 90% sequence identity to the amino acid sequence of any one of SEQ ID NOS:9 and 18, or that has up to two amino acid substitutions relative to the amino acid sequence of any one of SEQ ID NOS:9 and 18.

In some embodiments, the first binding region that specifically binds to TREM2 comprises two, three, four, five, or all six of (a)-(f). In some embodiments, the first binding region comprises the CDR-H1 of (a), the CDR-H2 of (b), and the CDR-H3 of (c). In some embodiments, the first binding region comprises the CDR-L1 of (d), the CDR-L2 of (e), and the CDR-L3 of (f). In some embodiments, a CDR having up to two amino acid substitutions has one amino acid substitution relative to the reference sequence. In some embodiments, a CDR having up to two amino acid substitutions has two amino acid substitutions relative to the reference sequence. In some embodiments, the up to two amino acid substitutions are conservative substitutions.

In some embodiments, the first binding region comprises one or more CDRs selected from the group consisting of:
(a) a CDR-H1 sequence comprising the amino acid sequence of any one of SEQ ID NOS:4 and 12;
(b) a CDR-H2 sequence comprising the amino acid sequence of any one of SEQ ID NOS:5, 13, and 25;
(c) a CDR-H3 sequence comprising the amino acid sequence of any one of SEQ ID NOS:6, 14, and 17;
(d) a CDR-L1 sequence comprising the amino acid sequence of any one of SEQ ID NOS:7 and 23;
(e) a CDR-L2 sequence comprising the amino acid sequence of SEQ ID NO:8; and
(f) a CDR-L3 sequence comprising the amino acid sequence of any one of SEQ ID NOS:9 and 18.

In some embodiments the first binding region comprises two, three, four, five, or all six of (a)-(f). In some embodiments, the first binding region comprises the CDR-H1 of (a), the CDR-H2 of (b), and the CDR-H3 of (c). In some embodiments, the first binding region comprises the CDR-L1 of (d), the CDR-L2 of (e), and the CDR-L3 of (f).

In some embodiments, the first binding region comprises:
(a) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:18; or
(b) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:17, a CDR-L1 comprising the amino acid sequence of SEQ ID NO:23, a CDR-L2 comprising the amino acid sequence of SEQ ID NO:8, and a CDR-L3 comprising the amino acid sequence of SEQ ID NO:18; or
(c) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO:17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:18; or
(d) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO:17, a CDR-L1 comprising the amino acid sequence of SEQ ID NO:23, a CDR-L2 comprising the amino acid sequence of SEQ ID NO:8, and a CDR-L3 comprising the amino acid sequence of SEQ ID NO:18; or
(e) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:6, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9; or
(f) a CDR-H1 comprising the amino acid sequence of SEQ ID NO:12, a CDR-H2 comprising the amino acid sequence of SEQ ID NO:13, a CDR-H3 comprising the amino acid sequence of SEQ ID NO:14, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9; or
(g) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO:17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9.

In some embodiments, the first binding region comprises a heavy chain variable region comprising an amino acid sequence that has at least 85%, 90%, 91%, 92%, 93%, 94%, 95%, 96%, 97%, 98%, or 99% sequence identity to any one of SEQ ID NOS:2, 10, 15, 19, 21, 24, and 26. In some embodiments, the first binding region comprises a heavy chain variable region comprising the amino acid sequence of any one of SEQ ID NOS:2, 10, 15, 19, 21, 24, and 26.

In some embodiments, the first binding region comprises a light chain variable region comprising an amino acid sequence that has at least 85%, 90%, 91%, 92%, 93%, 94%, 95%, 96%, 97%, 98%, or 99% sequence identity to any one of SEQ ID NOS:3, 11, 16, 20, 22, and 27. In some embodiments, the first binding region comprises a light chain variable region comprising the amino acid sequence of any one of SEQ ID NOS:3, 11, 16, 20, 22, and 27.

In some embodiments, the first binding region comprises: a heavy chain variable region comprising an amino acid sequence that has at least 85%, 90%, 91%, 92%, 93%, 94%, 95%, 96%, 97%, 98%, or 99% sequence identity to any one of SEQ ID NOS:2, 10, 15, 19, 21, 24, and 26, and a light chain variable region comprising an amino acid sequence that has at least 85%, 90%, 91%, 92%, 93%, 94%, 95%, 96%, 97%, 98%, or 99% sequence identity to any one of SEQ ID NOS:3, 11, 16, 20, 22, and 27. In some embodiments, the first binding region comprises: a heavy chain variable region comprising the amino acid sequence of any one of SEQ ID NOS:2, 10, 15, 19, 21, 24, and 26, and a light chain variable region comprising the amino acid sequence of any one of SEQ ID NOS:3, 11, 16, 20, 22, and 27.

In some embodiments, the first binding region comprises:
(a) a V_{H} sequence that has at least 85% sequence identity to SEQ ID NO:2 and a V_{L} sequence has at least 85% sequence identity to SEQ ID NO:3; or
(b) a V_{H} sequence that has at least 85% sequence identity to SEQ ID NO:10 and a V_{L} sequence has at least 85% sequence identity to SEQ ID NO:11; or
(c) a V_{H} sequence that has at least 85% sequence identity to SEQ ID NO:15 and a V_{L} sequence has at least 85% sequence identity to SEQ ID NO:16; or
(d) a V_{H} sequence that has at least 85% sequence identity to SEQ ID NO:19 and a V_{L} sequence has at least 85% sequence identity to SEQ ID NO:20; or
(e) a V_{H} sequence that has at least 85% sequence identity to SEQ ID NO:21 and a V_{L} sequence has at least 85% sequence identity to SEQ ID NO:20; or
(f) a V_{H} sequence that has at least 85% sequence identity to SEQ ID NO:19 and a V_{L} sequence has at least 85% sequence identity to SEQ ID NO:22; or
(g) a V_{H} sequence that has at least 85% sequence identity to SEQ ID NO:21 and a V_{L} sequence has at least 85% sequence identity to SEQ ID NO:22; or
(h) a V_{H} sequence that has at least 85% sequence identity to SEQ ID NO:24 and a V_{L} sequence has at least 85% sequence identity to SEQ ID NO:20; or
(i) a V_{H} sequence that has at least 85% sequence identity to SEQ ID NO:26 and a V_{L} sequence has at least 85% sequence identity to SEQ ID NO:20; or
(j) a V_{H} sequence that has at least 85% sequence identity to SEQ ID NO:24 and a V_{L} sequence has at least 85% sequence identity to SEQ ID NO:22; or
(k) a V_{H} sequence that has at least 85% sequence identity to SEQ ID NO:26 and a V_{L} sequence has at least 85% sequence identity to SEQ ID NO:22; or
(l) a V_{H} sequence that has at least 85% sequence identity to SEQ ID NO:24 and a V_{L} sequence has at least 85% sequence identity to SEQ ID NO:27.

In some embodiments, the first binding region comprises one or more sequences that are encompassed by a consensus sequence disclosed herein. As a non-limiting example, consensus sequences can be identified by aligning heavy chain or light chain sequences (e.g., CDRs) for antibodies that are from the same (or similar) germlines. In some embodiments, consensus sequences may be generated from antibodies that contain sequences that are of the same (or similar) length and/or have at least one highly similar CDR (e.g., a highly similar CDR3). In some embodiments, such sequences in these antibodies may be aligned and compared to identify conserved amino acids or motifs (i.e., where alteration in sequences may alter protein function) and/or regions where variation occurs the sequences (i.e., where variation of sequence is not likely to significantly affect protein function). Alternatively, consensus sequences can be identified by aligning heavy chain or light chain sequences (e.g., CDRs) for antibodies that bind to the same or similar (e.g., overlapping) epitopes to determine conserved amino acids or motifs (i.e., where alteration in sequences may alter protein function) and regions where variation occurs in alignment of sequences (i.e., where variation of sequence is not likely to significantly affect protein function). In some embodiments, one or more consensus sequences can be identified for antibodies that recognize the same or similar epitope as an anti-TREM2 binding molecule as disclosed herein. Exemplary consensus sequences include SEQ ID NOS:28-32. In the consensus sequences of SEQ ID NOS:28-32, the capitalized letter represents an amino acid residue that is absolutely conserved among the aligned sequences (e.g., aligned CDR sequences), while an “X” or a Greek letter (e.g., “α,” “β,” “γ,” “δ,” “ε,” or “φ”) represents an amino acid residue that is not absolutely conserved among the aligned sequences. It will be appreciated that, when selecting an amino acid to insert at a position marked by an “X” or by a Greek letter, in some embodiments the amino acid is selected from those amino acids found at the corresponding position in the aligned sequences.

Thus, in some embodiments, the first binding region comprises:
(a) a CDR-H1 sequence comprising the sequence of G-F-T-F-T-α₆-F-Y-M-S (SEQ ID NO:28), wherein α₆ is D or N;
(b) a CDR-H2 sequence comprising the sequence of V-I-R-N-β₅-β₆-N-β₈-Y-T-β₁₁-β₁₂-Y-N-P-S-V-K-G (SEQ ID NO:29), wherein β₅ is K or R; β₆ is A or P; β₈ is G or A; β₁₁ is A or T; and β₁₂ is G or D;
(c) a CDR-H3 sequence comprising the sequence of γ₁-R-L-γ₄-Y-G-F-D-Y (SEQ ID NO:30), wherein γ₁ is A or T; and γ₄ is T or S;
(d) a CDR-L1 sequence comprising the sequence of Q-S-S-K-S-L-L-H-S-δ₁₀-G-K-T-Y-L-N(SEQ ID NO:31), wherein δ₁₀ is N or T;
(e) a CDR-L2 sequence comprising the sequence of WMSTRAS (SEQ ID NO:8); and
(f) a CDR-L3 sequence comprising the sequence of Q-Q-F-L-E-ϕ₆-P-F-T (SEQ ID NO:32), wherein ϕ₆ is Y or F.

In some embodiments, the first binding region comprises a CDR-H1 sequence that is selected from SEQ ID NOS:4 and 12. In some embodiments, the first binding region comprises a CDR-H2 sequence that is selected from SEQ ID NOS:5, 13, and 25. In some embodiments, the first binding region comprises a CDR-H3 sequence that is selected from SEQ ID NOS:6, 14, and 17. In some embodiments, the first binding region comprises a CDR-L1 sequence that is selected from SEQ ID NOS:7 and 23. In some embodiments, the first binding region comprises a CDR-L3 sequence is selected from SEQ ID NOS:9 and 18.

In some embodiments, the first binding region comprises a heavy chain variable region comprising an amino acid sequence that has at least 85% sequence identity (e.g., at least 90%, 91%, 92%, 93%, 94%, 95%, 96%, 97%, 98%, or 99% sequence identity) to any one of SEQ ID NOS:2, 10, 15, 19, 21, 24, and 26. In some embodiments, the first binding region comprises a heavy chain variable region comprising the amino acid sequence of any one of SEQ ID NOS:2, 10, 15, 19, 21, 24, and 26.

In some embodiments, the first binding region comprises a light chain variable region comprising an amino acid sequence that has at least 85% sequence identity (e.g., at least 90%, 91%, 92%, 93%, 94%, 95%, 96%, 97%, 98%, or 99% sequence identity) to any one of SEQ ID NOS:3, 11, 16, 20, 22, and 27. In some embodiments, the first binding region comprises a light chain variable region comprising the amino acid sequence of any one of SEQ ID NOS:3, 11, 16, 20, 22, and 27.

### Clone CL0020188 and Associated Antibodies

In some embodiments, the first binding region comprises a CDR-H1 sequence comprising the amino acid sequence of SEQ ID NO:4, a CDR-H2 sequence comprising the amino acid sequence of SEQ ID NO:5, a CDR-H3 sequence comprising the amino acid sequence of SEQ ID NO:17, a CDR-L1 sequence comprising the amino acid sequence of SEQ ID NO:7, a CDR-L2 sequence comprising the amino acid sequence of SEQ ID NO:8, and a CDR-L3 sequence comprising the amino acid sequence of SEQ ID NO:18.

In some embodiments, the first binding region comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:15. In some embodiments, the first binding region comprises a heavy chain variable region comprising the amino acid sequence of SEQ ID NO:15.

In some embodiments, the first binding region comprises a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:16. In some embodiments, the first binding region comprises a light chain variable region comprising the amino acid sequence of SEQ ID NO:16.

In some embodiments, the first binding region comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:15 and a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:16. In some embodiments, the first binding region comprises a heavy chain variable region comprising the amino acid sequence of SEQ ID NO:15 and a light chain variable region comprising the amino acid sequence of SEQ ID NO:16.

In some embodiments, the first binding region comprises a heavy chain variable region that comprises a heavy chain CDR1-3 comprising the amino acid sequences of SEQ ID NOS:4, 5, and 17, respectively, and that has at least 85% sequence identity (e.g., at least 90%, 91%, 92%, 93%, 94%, 95%, 96%, 97%, 98%, or 99% sequence identity) to SEQ ID NO:15. In some embodiments, the first binding region comprises a light chain variable region that comprises a light chain CDR1-3 comprising the amino acid sequences of SEQ ID NOS:7, 8, and 18, respectively, and that has at least 85% sequence identity (e.g., at least 90%, 91%, 92%, 93%, 94%, 95%, 96%, 97%, 98%, or 99% sequence identity) to SEQ ID NO:16.

In some embodiments, the first binding region comprises a CDR-H1 sequence comprising the amino acid sequence of SEQ ID NO:4, a CDR-H2 sequence comprising the amino acid sequence of SEQ ID NO:25, a CDR-H3 sequence comprising the amino acid sequence of SEQ ID NO:17, a CDR-L1 sequence comprising the amino acid sequence of SEQ ID NO:23, a CDR-L2 sequence comprising the amino acid sequence of SEQ ID NO:8, and a CDR-L3 sequence comprising the amino acid sequence of SEQ ID NO:18.

In some embodiments, the first binding region comprises a heavy chain variable region comprising an amino acid sequence that has at least 85% sequence identity (e.g., at least 90%, 91%, 92%, 93%, 94%, 95%, 96%, 97%, 98%, or 99% sequence identity, such as at least 90%, 95%, or 97% sequence identity) to SEQ ID NO:24. In some embodiments, the first binding region comprises a heavy chain variable region comprising the amino acid sequence of SEQ ID NO:24.

In some embodiments, the first binding region comprises a light chain variable region comprising an amino acid sequence that has at least 85% sequence identity (e.g., at least 90%, 91%, 92%, 93%, 94%, 95%, 96%, 97%, 98%, or 99% sequence identity, such as at least 90%, 95%, or 97% sequence identity) to SEQ ID NO:22. In some embodiments, the first binding region comprises a light chain variable region comprising the amino acid sequence of SEQ ID NO:22.

In some embodiments, the first binding region comprises a heavy chain variable region comprising an amino acid sequence that has at least 85% sequence identity (e.g., at least 90%, 91%, 92%, 93%, 94%, 95%, 96%, 97%, 98%, or 99% sequence identity, such as at least 90%, 95%, or 97% sequence identity) to SEQ ID NO:24 and a light chain variable region comprising an amino acid sequence that has at least 85% sequence identity (e.g., at least 90%, 91%, 92%, 93%, 94%, 95%, 96%, 97%, 98%, or 99% sequence identity, such as at least 90%, 95%, or 97% sequence identity) to SEQ ID NO:22. In some embodiments, the first binding region comprises a heavy chain variable region comprising the amino acid sequence of SEQ ID NO:24 and a light chain variable region comprising the amino acid sequence of SEQ ID NO:22.

In some embodiments, the first binding region comprises a heavy chain variable region that comprises a heavy chain CDR1-3 comprising the amino acid sequences of SEQ ID NOS:4, 25, and 17, respectively, and that has at least 85% sequence identity (e.g., at least 90%, 91%, 92%, 93%, 94%, 95%, 96%, 97%, 98%, or 99% sequence identity, such as at least 90%, 95%, or 97% sequence identity) to SEQ ID NO:24. In some embodiments the first binding region comprises a light chain variable region that comprises a light chain CDR1-3 comprising the amino acid sequences of SEQ ID NOS:23, 8, and 18, respectively, and that has at least 85% sequence identity (e.g., at least 90%, 91%, 92%, 93%, 94%, 95%, 96%, 97%, 98%, or 99% sequence identity, such as at least 90%, 95%, or 97% sequence identity) to SEQ ID NO:22.

In some embodiments, the first binding region comprises a CDR-H1 sequence comprising the amino acid sequence of SEQ ID NO:4, a CDR-H2 sequence comprising the amino acid sequence of SEQ ID NO:25, a CDR-H3 sequence comprising the amino acid sequence of SEQ ID NO:17, a CDR-L1 sequence comprising the amino acid sequence of SEQ ID NO:7, a CDR-L2 sequence comprising the amino acid sequence of SEQ ID NO:8, and a CDR-L3 sequence comprising the amino acid sequence of SEQ ID NO:9.

In some embodiments, the first binding region comprises a heavy chain variable region comprising an amino acid sequence that has at least 85% sequence identity (e.g., at least 90%, 91%, 92%, 93%, 94%, 95%, 96%, 97%, 98%, or 99% sequence identity, such as at least 90%, 95%, or 97% sequence identity) to SEQ ID NO:24. In some embodiments, the first binding region comprises a heavy chain variable region comprising the amino acid sequence of SEQ ID NO:24.

In some embodiments, the first binding region comprises a light chain variable region comprising an amino acid sequence that has at least 85% sequence identity (e.g., at least 90%, 91%, 92%, 93%, 94%, 95%, 96%, 97%, 98%, or 99% sequence identity, such as at least 90%, 95%, or 97% sequence identity) to SEQ ID NO:27. In some embodiments, the first binding region comprises a light chain variable region comprising the amino acid sequence of SEQ ID NO:27.

In some embodiments, the first binding region comprises a heavy chain variable region comprising an amino acid sequence that has at least 85% sequence identity (e.g., at least 90%, 91%, 92%, 93%, 94%, 95%, 96%, 97%, 98%, or 99% sequence identity, such as at least 90%, 95%, or 97% sequence identity) to SEQ ID NO:24 and a light chain variable region comprising an amino acid sequence that has at least 85% sequence identity (e.g., at least 90%, 91%, 92%, 93%, 94%, 95%, 96%, 97%, 98%, or 99% sequence identity, such as at least 90%, 95%, or 97% sequence identity) to SEQ ID NO:27. In some embodiments, the first binding region comprises a heavy chain variable region comprising the amino acid sequence of SEQ ID NO:24 and a light chain variable region comprising the amino acid sequence of SEQ ID NO:27.

In some embodiments, the first binding region comprises a heavy chain variable region that comprises a heavy chain CDR1-3 comprising the amino acid sequences of SEQ ID NOS:4, 25, and 17, respectively, and that has at least 85% sequence identity (e.g., at least 90%, 91%, 92%, 93%, 94%, 95%, 96%, 97%, 98%, or 99% sequence identity, such as at least 90%, 95%, or 97% sequence identity) to SEQ ID NO:24. In some embodiments, the first binding region comprises a light chain variable region that comprises a light chain CDR1-3 comprising the amino acid sequences of SEQ ID NOS:7, 8, and 9, respectively, and that has at least 85% sequence identity (e.g., at least 90%, 91%, 92%, 93%, 94%, 95%, 96%, 97%, 98%, or 99% sequence identity, such as at least 90%, 95%, or 97% sequence identity) to SEQ ID NO:27.

### Clone CL0020306

In some embodiments, the first binding region comprises a CDR-H1 sequence comprising the amino acid sequence of SEQ ID NO:4, a CDR-H2 sequence comprising the amino acid sequence of SEQ ID NO:5, a CDR-H3 sequence comprising the amino acid sequence of SEQ ID NO:6, a CDR-L1 sequence comprising the amino acid sequence of SEQ ID NO:7, a CDR-L2 sequence comprising the amino acid sequence of SEQ ID NO:8, and a CDR-L3 sequence comprising the amino acid sequence of SEQ ID NO:9.

In some embodiments, the first binding region comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:2. In some embodiments, the first binding region comprises a heavy chain variable region comprising the amino acid sequence of SEQ ID NO:2.

In some embodiments, the first binding region comprises a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:3. In some embodiments, the first binding region comprises a light chain variable region comprising the amino acid sequence of SEQ ID NO:3.

In some embodiments, the first binding region comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:2 and a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:3. In some embodiments, the first binding region comprises a heavy chain variable region comprising the amino acid sequence of SEQ ID NO:2 and a light chain variable region comprising the amino acid sequence of SEQ ID NO:3.

In some embodiments, the first binding region comprises a heavy chain variable region that comprises a heavy chain CDR1-3 comprising the amino acid sequences of SEQ ID NOS:4, 5, and 6, respectively, and that has at least 85% sequence identity (e.g., at least 90%, 91%, 92%, 93%, 94%, 95%, 96%, 97%, 98%, or 99% sequence identity) to SEQ ID NO:2. In some embodiments, the first binding region comprises a light chain variable region that comprises a light chain CDR1-3 comprising the amino acid sequences of SEQ ID NOS:7, 8, and 9, respectively, and that has at least 85% sequence identity (e.g., at least 90%, 91%, 92%, 93%, 94%, 95%, 96%, 97%, 98%, or 99% sequence identity) to SEQ ID NO:3.

### Clone CL0020307

In some embodiments, the first binding region comprises a CDR-H1 sequence comprising the amino acid sequence of SEQ ID NO:12, a CDR-H2 sequence comprising the amino acid sequence of SEQ ID NO:13, a CDR-H3 sequence comprising the amino acid sequence of SEQ ID NO:14, a CDR-L1 sequence comprising the amino acid sequence of SEQ ID NO:7, a CDR-L2 sequence comprising the amino acid sequence of SEQ ID NO:8, and a CDR-L3 sequence comprising the amino acid sequence of SEQ ID NO:9.

In some embodiments, the first binding region comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:10. In some embodiments, the first binding region comprises a heavy chain variable region comprising the amino acid sequence of SEQ ID NO:10.

In some embodiments, the first binding region comprises a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:11. In some embodiments, the first binding region comprises a light chain variable region comprising the amino acid sequence of SEQ ID NO:11.

In some embodiments, the first binding region comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:10 and a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:11. In some embodiments, the first binding region comprises a heavy chain variable region comprising the amino acid sequence of SEQ ID NO:10 and a light chain variable region comprising the amino acid sequence of SEQ ID NO: 11.

In some embodiments, the first binding region comprises a heavy chain variable region that comprises a heavy chain CDR1-3 comprising the amino acid sequences of SEQ ID NOS:12, 13, and 14, respectively, and that has at least 85% sequence identity (e.g., at least 90%, 91%, 92%, 93%, 94%, 95%, 96%, 97%, 98%, or 99% sequence identity) to SEQ ID NO:10. In some embodiments, the first binding region comprises a light chain variable region that comprises a light chain CDR1-3 comprising the amino acid sequences of SEQ ID NOS:7, 8, and 9, respectively, and that has at least 85% sequence identity (e.g., at least 90%, 91%, 92%, 93%, 94%, 95%, 96%, 97%, 98%, or 99% sequence identity) to SEQ ID NO:11.

### Clone A

In some embodiments, the first binding region comprises one or more CDRs selected from the group consisting of:
(a) a heavy chain CDR1 (CDR-H1) comprising a sequence that has at least 90% sequence identity to the amino acid sequence of SYWIG (SEQ ID NO:114), or that has up to two amino acid substitutions relative to the amino acid sequence of SYWIG (SEQ ID NO:114);
(b) a heavy chain CDR2 (CDR-H2) comprising a sequence that has at least 90% sequence identity to the amino acid sequence of IIYPGDADARYSPSFQG (SEQ ID NO:115 or that has u to two amino acid substitutions relative to the amino acid sequence of

(SEQ ID NO: 115)
IIYPGDADARYSPSFQG;

(c) a heavy chain CDR3 (CDR-H3) comprising a sequence that has at least 90% sequence identity to the amino acid sequence of RRQGIFGDALDF (SEQ ID NO:116), or that has up to two amino acid substitutions relative to the amino acid sequence of

(SEQ ID NO: 116)
RRQGIFGDALDF;

(d) a light chain CDR1 (CDR-L1) comprising a sequence that has at least 90% sequence identity to the amino acid sequence of RASQSVSSNLA (SEQ ID NO:110), or that has up to two amino acid substitutions relative to the amino acid sequence of RASQSVSSNLA (SEQ ID NO:110);
(e) a light chain CDR2 (CDR-L2) comprising a sequence that has at least 90% sequence identity to the amino acid sequence of GASTRAT (SEQ ID NO:111), or that has up to two amino acid substitutions relative to the amino acid sequence of GASTRAT (SEQ ID NO:111); and
(f) a light chain CDR3 (CDR-L3) comprising a sequence that has at least 90% sequence identity to the amino acid sequence of LQDNNFPPT (SEQ ID NO:112), or that has up to two amino acid substitutions relative to the amino acid sequence of LQDNNFPPT (SEQ ID NO:112).

In some embodiments, the first binding region comprises two, three, four, five, or all six of (a)-(f). In some embodiments, the first binding region comprises the CDR-H1 of (a), the CDR-H2 of (b), and the CDR-H3 of (c). In some embodiments, the first binding region comprises the CDR-L1 of (d), the CDR-L2 of (e), and the CDR-L3 of (f). In some embodiments, a CDR having up to two amino acid substitutions has one amino acid substitution relative to the reference sequence. In some embodiments, a CDR having up to two amino acid substitutions has two amino acid substitutions relative to the reference sequence. In some embodiments, the up to two amino acid substitutions are conservative substitutions.

In some embodiments, the first binding region comprises one or more CDRs selected from the group consisting of:
(a) a CDR-H1 sequence comprising the amino acid sequence of SEQ ID NO:114;
(b) a CDR-H2 sequence comprising the amino acid sequence of SEQ ID NO:115;
(c) a CDR-H3 sequence comprising the amino acid sequence of SEQ ID NO:116;
(d) a CDR-L1 sequence comprising the amino acid sequence of SEQ ID NO:110;
(e) a CDR-L2 sequence comprising the amino acid sequence of SEQ ID NO:111; and
(f) a CDR-L3 sequence comprising the amino acid sequence of SEQ ID NO: 112.

In some embodiments, the first binding region comprises two, three, four, five, or all six of (a)-(f). In some embodiments, the first binding region comprises the CDR-H1 of (a), the CDR-H2 of (b), and the CDR-H3 of (c). In some embodiments, the first binding region comprises the CDR-L1 of (d), the CDR-L2 of (e), and the CDR-L3 of (f).

In some embodiments, the first binding region comprises a CDR-H1 comprising the amino acid sequence of SEQ ID NO:114, a CDR-H2 comprising the amino acid sequence of SEQ ID NO:115, a CDR-H3 comprising the amino acid sequence of SEQ ID NO:116, a CDR-L1 comprising the amino acid sequence of SEQ ID NO:110, a CDR-L2 comprising the amino acid sequence of SEQ ID NO:111, and a CDR-L3 comprising the amino acid sequence of SEQ ID NO:112.

In some embodiments, the first binding region comprises a heavy chain variable region comprising an amino acid sequence that has at least 85%, 90%, 91%, 92%, 93%, 94%, 95%, 96%, 97%, 98%, or 99% sequence identity to SEQ ID NO:113. In some embodiments, the first binding region comprises a heavy chain variable region comprising the amino acid sequence of SEQ ID NO:113.

In some embodiments, the first binding region comprises a light chain variable region comprising an amino acid sequence that has at least 85%, 90%, 91%, 92%, 93%, 94%, 95%, 96%, 97%, 98%, or 99% sequence identity to SEQ ID NO:109. In some embodiments, the first binding region comprises a light chain variable region comprising the amino acid sequence of SEQ ID NO:109.

In some embodiments, the first binding region comprises: a heavy chain variable region comprising an amino acid sequence that has at least 85%, 90%, 91%, 92%, 93%, 94%, 95%, 96%, 97%, 98%, or 99% sequence identity to SEQ ID NO:113, and a light chain variable region comprising an amino acid sequence that has at least 85%, 90%, 91%, 92%, 93%, 94%, 95%, 96%, 97%, 98%, or 99% sequence identity to SEQ ID NO:109. In some embodiments, the first binding region comprises: a heavy chain variable region comprising the amino acid sequence of SEQ ID NO:113, and a light chain variable region comprising the amino acid sequence of SEQ ID NO:109.

In some embodiments, the first binding region comprises a heavy chain variable region that comprises a heavy chain CDR1-3 comprising the amino acid sequences of SEQ ID NOS:114, 115, and 116, respectively, and that has at least 85% sequence identity (e.g., at least 90%, 91%, 92%, 93%, 94%, 95%, 96%, 97%, 98%, or 99% sequence identity) to SEQ ID NO:113. In some embodiments, the first binding region comprises a light chain variable region that comprises a light chain CDR1-3 comprising the amino acid sequences of SEQ ID NOS:110, 111, and 112, respectively, and that has at least 85% sequence identity (e.g., at least 90%, 91%, 92%, 93%, 94%, 95%, 96%, 97%, 98%, or 99% sequence identity) to SEQ ID NO:109.

### Clone B

In some embodiments, the first binding region comprises one or more CDRs selected from the group consisting of:
(a) a heavy chain CDR1 (CDR-H1) comprising a sequence that has at least 90% sequence identity to the amino acid sequence NYWIA (SEQ ID NO:122), or that has up to two amino acid substitutions relative to the amino acid sequence of NYWIA (SEQ ID NO:122);
(b) a heavy chain CDR2 (CDR-H2) comprising a sequence that has at least 90% sequence identity to the amino acid sequence of IIYPGDSDTRYSPSFQG (SEQ ID NO:123), or that has up to two amino acid substitutions relative to the amino acid sequence of

(SEQ ID NO: 123)
IIYPGDSDTRYSPSFQG;

(c) a heavy chain CDR3 (CDR-H3) comprising a sequence that has at least 90% sequence identity to the amino acid sequence of QRTFYYDSSGYFDY (SEQ ID NO:124), or that has up to two amino acid substitutions relative to the amino acid sequence of

(SEQ ID NO: 124)
QRTFYYDSSGYFDY;

(d) a light chain CDR1 (CDR-L1) comprising a sequence that has at least 90% sequence identity to the amino acid sequence of RASQGISNWLA (SEQ ID NO:118), or that has up to two amino acid substitutions relative to the amino acid sequence of RASQGISNWLA (SEQ ID NO:118);
(e) a light chain CDR2 (CDR-L2) comprising a sequence that has at least 90% sequence identity to the amino acid sequence of AASSLQV (SEQ ID NO:119), or that has up to two amino acid substitutions relative to the amino acid sequence of AASSLQV (SEQ ID NO:119); and
(f) a light chain CDR3 (CDR-L3) comprising a sequence that has at least 90% sequence identity to the amino acid sequence of QQADSFPRN (SEQ ID NO:120), or that has up to two amino acid substitutions relative to the amino acid sequence of QQADSFPRN (SEQ ID NO:120).

In some embodiments, the first binding region comprises two, three, four, five, or all six of (a)-(f). In some embodiments, the first binding region comprises the CDR-H1 of (a), the CDR-H2 of (b), and the CDR-H3 of (c). In some embodiments, the first binding region comprises the CDR-L1 of (d), the CDR-L2 of (e), and the CDR-L3 of (f). In some embodiments, a CDR having up to two amino acid substitutions has one amino acid substitution relative to the reference sequence. In some embodiments, a CDR having up to two amino acid substitutions has two amino acid substitutions relative to the reference sequence. In some embodiments, the up to two amino acid substitutions are conservative substitutions.

In some embodiments, the first binding region comprises one or more CDRs selected from the group consisting of:
(a) a CDR-H1 sequence comprising the amino acid sequence of SEQ ID NO:122;
(b) a CDR-H2 sequence comprising the amino acid sequence of SEQ ID NO:123;
(c) a CDR-H3 sequence comprising the amino acid sequence of SEQ ID NO:124;
(d) a CDR-L1 sequence comprising the amino acid sequence of SEQ ID NO:118;
(e) a CDR-L2 sequence comprising the amino acid sequence of SEQ ID NO:119; and
(f) a CDR-L3 sequence comprising the amino acid sequence of SEQ ID NO:120.

In some embodiments, the first binding region comprises two, three, four, five, or all six of (a)-(f). In some embodiments, the first binding region comprises the CDR-H1 of (a), the CDR-H2 of (b), and the CDR-H3 of (c). In some embodiments, the first binding region comprises the CDR-L1 of (d), the CDR-L2 of (e), and the CDR-L3 of (f).

In some embodiments, the first binding region comprises a CDR-H1 comprising the amino acid sequence of SEQ ID NO:122, a CDR-H2 comprising the amino acid sequence of SEQ ID NO:123, a CDR-H3 comprising the amino acid sequence of SEQ ID NO:124, a CDR-L1 comprising the amino acid sequence of SEQ ID NO:118, a CDR-L2 comprising the amino acid sequence of SEQ ID NO:119, and a CDR-L3 comprising the amino acid sequence of SEQ ID NO:120.

In some embodiments, the first binding region comprises a heavy chain variable region comprising an amino acid sequence that has at least 85%, 90%, 91%, 92%, 93%, 94%, 95%, 96%, 97%, 98%, or 99% sequence identity to SEQ ID NO:121. In some embodiments, the first binding region comprises a heavy chain variable region comprising the amino acid sequence of SEQ ID NO:121.

In some embodiments, the first binding region comprises a light chain variable region comprising an amino acid sequence that has at least 85%, 90%, 91%, 92%, 93%, 94%, 95%, 96%, 97%, 98%, or 99% sequence identity to SEQ ID NO:117. In some embodiments, the first binding region comprises a light chain variable region comprising the amino acid sequence of SEQ ID NO:117.

In some embodiments, the first binding region comprises: a heavy chain variable region comprising an amino acid sequence that has at least 85%, 90%, 91%, 92%, 93%, 94%, 95%, 96%, 97%, 98%, or 99% sequence identity to SEQ ID NO:121, and a light chain variable region comprising an amino acid sequence that has at least 85%, 90%, 91%, 92%, 93%, 94%, 95%, 96%, 97%, 98%, or 99% sequence identity to SEQ ID NO:117. In some embodiments, the first binding region comprises: a heavy chain variable region comprising the amino acid sequence of SEQ ID NO:121, and a light chain variable region comprising the amino acid sequence of SEQ ID NO:117.

In some embodiments, the first binding region comprises a heavy chain variable region that comprises a heavy chain CDR1-3 comprising the amino acid sequences of SEQ ID NOS:122, 123, and 124, respectively, and that has at least 85% sequence identity (e.g., at least 90%, 91%, 92%, 93%, 94%, 95%, 96%, 97%, 98%, or 99% sequence identity) to SEQ ID NO:121. In some embodiments, the first binding region comprises a light chain variable region that comprises a light chain CDR1-3 comprising the amino acid sequences of SEQ ID NOS:118, 119, and 120, respectively, and that has at least 85% sequence identity (e.g., at least 90%, 91%, 92%, 93%, 94%, 95%, 96%, 97%, 98%, or 99% sequence identity) to SEQ ID NO:117.

### Clone C

In some embodiments, the first binding region comprises one or more CDRs selected from the group consisting of:
(a) a heavy chain CDR1 (CDR-H1) comprising a sequence that has at least 90% sequence identity to the amino acid sequence SYWIA (SEQ ID NO:130), or that has up to two amino acid substitutions relative to the amino acid sequence of SYWIA (SEQ ID NO:130);
(b) a heavy chain CDR2 (CDR-H2) comprising a sequence that has at least 90% sequence identity to the amino acid sequence of IIYPGDSDTRYSPSFQG (SEQ ID NO:123, or that has u to two amino acid substitutions relative to the amino acid sequence of

(SEQ ID NO: 123)
IIYPGDSDTRYSPSFQG;

(c) a heavy chain CDR3 (CDR-H3) comprising a sequence that has at least 90% sequence identity to the amino acid sequence of QRTFYYDSSDYFDY (SEQ ID NO:131), or that has up to two amino acid substitutions relative to the amino acid sequence of

(SEQ ID NO: 131)
QRTFYYDSSDYFDY;

(d) a light chain CDR1 (CDR-L1) comprising a sequence that has at least 90% sequence identity to the amino acid sequence of RASQGISSWLA (SEQ ID NO:126), or that has up to two amino acid substitutions relative to the amino acid sequence of RASQGISSWLA (SEQ ID NO:126);
(e) a light chain CDR2 (CDR-L2) comprising a sequence that has at least 90% sequence identity to the amino acid sequence of AASSLQN (SEQ ID NO:127), or that has up to two amino acid substitutions relative to the amino acid sequence of AASSLQN (SEQ ID NO:127); and
(f) a light chain CDR3 (CDR-L3) comprising a sequence that has at least 90% sequence identity to the amino acid sequence of QQADSFPRT (SEQ ID NO:128), or that has up to two amino acid substitutions relative to the amino acid sequence of QQADSFPRT (SEQ ID NO:128).

In some embodiments, the first binding region comprises two, three, four, five, or all six of (a)-(f). In some embodiments, the first binding region comprises the CDR-H1 of (a), the CDR-H2 of (b), and the CDR-H3 of (c). In some embodiments, the first binding region comprises the CDR-L1 of (d), the CDR-L2 of (e), and the CDR-L3 of (f). In some embodiments, a CDR having up to two amino acid substitutions has one amino acid substitution relative to the reference sequence. In some embodiments, a CDR having up to two amino acid substitutions has two amino acid substitutions relative to the reference sequence. In some embodiments, the up to two amino acid substitutions are conservative substitutions.

In some embodiments, the first binding region comprises one or more CDRs selected from the group consisting of:
(a) a CDR-H1 sequence comprising the amino acid sequence of SEQ ID NO:130;
(b) a CDR-H2 sequence comprising the amino acid sequence of SEQ ID NO:123;
(c) a CDR-H3 sequence comprising the amino acid sequence of SEQ ID NO:131;
(d) a CDR-L1 sequence comprising the amino acid sequence of SEQ ID NO:126;
(e) a CDR-L2 sequence comprising the amino acid sequence of SEQ ID NO:127; and
(f) a CDR-L3 sequence comprising the amino acid sequence of SEQ ID NO:128.

In some embodiments, the first binding region comprises two, three, four, five, or all six of (a)-(f). In some embodiments, the first binding region comprises the CDR-H1 of (a), the CDR-H2 of (b), and the CDR-H3 of (c). In some embodiments, the first binding region comprises the CDR-L1 of (d), the CDR-L2 of (e), and the CDR-L3 of (f).

In some embodiments, the first binding region comprises a CDR-H1 comprising the amino acid sequence of SEQ ID NO:130, a CDR-H2 comprising the amino acid sequence of SEQ ID NO:123, a CDR-H3 comprising the amino acid sequence of SEQ ID NO:131, a CDR-L1 comprising the amino acid sequence of SEQ ID NO:126, a CDR-L2 comprising the amino acid sequence of SEQ ID NO:127, and a CDR-L3 comprising the amino acid sequence of SEQ ID NO:128.

In some embodiments, the first binding region comprises a heavy chain variable region comprising an amino acid sequence that has at least 85%, 90%, 91%, 92%, 93%, 94%, 95%, 96%, 97%, 98%, or 99% sequence identity to SEQ ID NO:129. In some embodiments, the first binding region comprises a heavy chain variable region comprising the amino acid sequence of SEQ ID NO:129.

In some embodiments, the first binding region comprises a light chain variable region comprising an amino acid sequence that has at least 85%, 90%, 91%, 92%, 93%, 94%, 95%, 96%, 97%, 98%, or 99% sequence identity to SEQ ID NO:125. In some embodiments, the first binding region comprises a light chain variable region comprising the amino acid sequence of SEQ ID NO:125.

In some embodiments, the first binding region comprises: a heavy chain variable region comprising an amino acid sequence that has at least 85%, 90%, 91%, 92%, 93%, 94%, 95%, 96%, 97%, 98%, or 99% sequence identity to SEQ ID NO:129, and a light chain variable region comprising an amino acid sequence that has at least 85%, 90%, 91%, 92%, 93%, 94%, 95%, 96%, 97%, 98%, or 99% sequence identity to SEQ ID NO:125. In some embodiments, the first binding region comprises: a heavy chain variable region comprising the amino acid sequence of SEQ ID NO:129, and a light chain variable region comprising the amino acid sequence of SEQ ID NO:125.

In some embodiments, the first binding region comprises a heavy chain variable region that comprises a heavy chain CDR1-3 comprising the amino acid sequences of SEQ ID NOS:130, 123, and 131, respectively, and that has at least 85% sequence identity (e.g., at least 90%, 91%, 92%, 93%, 94%, 95%, 96%, 97%, 98%, or 99% sequence identity) to SEQ ID NO:129. In some embodiments, the first binding region comprises a light chain variable region that comprises a light chain CDR1-3 comprising the amino acid sequences of SEQ ID NOS:126, 127, and 128, respectively, and that has at least 85% sequence identity (e.g., at least 90%, 91%, 92%, 93%, 94%, 95%, 96%, 97%, 98%, or 99% sequence identity) to SEQ ID NO:125.

### Clone D

In some embodiments, the first binding region comprises one or more CDRs selected from the group consisting of:
(a) a heavy chain CDR1 (CDR-H1) comprising a sequence that has at least 90% sequence identity to the amino acid sequence YAFSSQWMN (SEQ ID NO:137), or that has up to two amino acid substitutions relative to the amino acid sequence of YAFSSQWMN (SEQ ID NO:137);
(b) a heavy chain CDR2 (CDR-H2) comprising a sequence that has at least 90% sequence identity to the amino acid sequence of RIYPGGGDTNYAGKFQG (SEQ ID NO:138), or that has up to two amino acid substitutions relative to the amino acid sequence of

(SEQ ID NO: 138)
RIYPGGGDTNYAGKFQG;

(c) a heavy chain CDR3 (CDR-H3) comprising a sequence that has at least 90% sequence identity to the amino acid sequence of ARLLRNQPGESYAMDY (SEQ ID NO:139), or that has up to two amino acid substitutions relative to the amino acid sequence of

(SEQ ID NO: 139)
ARLLRNQPGESYAMDY;

(d) a light chain CDR1 (CDR-L1) comprising a sequence that has at least 90% sequence identity to the amino acid sequence of RSSQSLVHSNRYTYLH (SEQ ID NO:133), or that has up to two amino acid substitutions relative to the amino acid sequence of

(SEQ ID NO: 133)
RSSQSLVHSNRYTYLH;

(e) a light chain CDR2 (CDR-L2) comprising a sequence that has at least 90% sequence identity to the amino acid sequence of KVSNRFS (SEQ ID NO:134), or that has up to two amino acid substitutions relative to the amino acid sequence of KVSNRFS (SEQ ID NO:134); and
(f) a light chain CDR3 (CDR-L3) comprising a sequence that has at least 90% sequence identity to the amino acid sequence of SQSTRVPYT (SEQ ID NO:135), or that has up to two amino acid substitutions relative to the amino acid sequence of SQSTRVPYT (SEQ ID NO:135).

In some embodiments, the first binding region comprises two, three, four, five, or all six of (a)-(f). In some embodiments, the first binding region comprises the CDR-H1 of (a), the CDR-H2 of (b), and the CDR-H3 of (c). In some embodiments, the first binding region comprises the CDR-L1 of (d), the CDR-L2 of (e), and the CDR-L3 of (f). In some embodiments, a CDR having up to two amino acid substitutions has one amino acid substitution relative to the reference sequence. In some embodiments, a CDR having up to two amino acid substitutions has two amino acid substitutions relative to the reference sequence. In some embodiments, the up to two amino acid substitutions are conservative substitutions.

In some embodiments, the first binding region comprises one or more CDRs selected from the group consisting of:
(a) a CDR-H1 sequence comprising the amino acid sequence of SEQ ID NO:137;
(b) a CDR-H2 sequence comprising the amino acid sequence of SEQ ID NO:138;
(c) a CDR-H3 sequence comprising the amino acid sequence of SEQ ID NO:139;
(d) a CDR-L1 sequence comprising the amino acid sequence of SEQ ID NO:133;
(e) a CDR-L2 sequence comprising the amino acid sequence of SEQ ID NO:134; and
(f) a CDR-L3 sequence comprising the amino acid sequence of SEQ ID NO:135.

In some embodiments, the first binding region comprises two, three, four, five, or all six of (a)-(f). In some embodiments, the first binding region comprises the CDR-H1 of (a), the CDR-H2 of (b), and the CDR-H3 of (c). In some embodiments, the first binding region comprises the CDR-L1 of (d), the CDR-L2 of (e), and the CDR-L3 of (f).

In some embodiments, the first binding region comprises a CDR-H1 comprising the amino acid sequence of SEQ ID NO:137, a CDR-H2 comprising the amino acid sequence of SEQ ID NO:138, a CDR-H3 comprising the amino acid sequence of SEQ ID NO:139, a CDR-L1 comprising the amino acid sequence of SEQ ID NO:133, a CDR-L2 comprising the amino acid sequence of SEQ ID NO:134, and a CDR-L3 comprising the amino acid sequence of SEQ ID NO:135.

In some embodiments, the first binding region comprises a heavy chain variable region comprising an amino acid sequence that has at least 85%, 90%, 91%, 92%, 93%, 94%, 95%, 96%, 97%, 98%, or 99% sequence identity to SEQ ID NO:136. In some embodiments, the first binding region comprises a heavy chain variable region comprising the amino acid sequence of SEQ ID NO:136.

In some embodiments, the first binding region comprises a light chain variable region comprising an amino acid sequence that has at least 85%, 90%, 91%, 92%, 93%, 94%, 95%, 96%, 97%, 98%, or 99% sequence identity to SEQ ID NO:132. In some embodiments, the first binding region comprises a light chain variable region comprising the amino acid sequence of SEQ ID NO:132.

In some embodiments, the first binding region comprises: a heavy chain variable region comprising an amino acid sequence that has at least 85%, 90%, 91%, 92%, 93%, 94%, 95%, 96%, 97%, 98%, or 99% sequence identity to SEQ ID NO:136, and a light chain variable region comprising an amino acid sequence that has at least 85%, 90%, 91%, 92%, 93%, 94%, 95%, 96%, 97%, 98%, or 99% sequence identity to SEQ ID NO:132. In some embodiments, the first binding region comprises: a heavy chain variable region comprising the amino acid sequence of SEQ ID NO:136, and a light chain variable region comprising the amino acid sequence of SEQ ID NO:132.

In some embodiments, the first binding region comprises a heavy chain variable region that comprises a heavy chain CDR1-3 comprising the amino acid sequences of SEQ ID NOS:137, 138, and 139, respectively, and that has at least 85% sequence identity (e.g., at least 90%, 91%, 92%, 93%, 94%, 95%, 96%, 97%, 98%, or 99% sequence identity) to SEQ ID NO:136. In some embodiments, the first binding region comprises a light chain variable region that comprises a light chain CDR1-3 comprising the amino acid sequences of SEQ ID NOS:133, 134, and 135, respectively, and that has at least 85% sequence identity (e.g., at least 90%, 91%, 92%, 93%, 94%, 95%, 96%, 97%, 98%, or 99% sequence identity) to SEQ ID NO:132.

### Binding Characteristics

In some embodiments, a molecule as described herein that specifically binds to a TREM2 protein binds to TREM2 that is expressed on a cell (e.g., a primary cell or cell line that endogenously expresses TREM2, such as human macrophages, or a primary cell or cell line that has been engineered to express TREM2). In some embodiments, a molecule that specifically binds to a TREM2 protein as described herein binds to purified or recombinant TREM2 protein of a portion thereof, or to a chimeric protein comprising TREM2 or a portion thereof (e.g., an Fc-fusion protein comprising TREM2 or an Fc-fusion protein comprising the ecto-domain of TREM2).

In some embodiments, a molecule that specifically binds to human TREM2 protein exhibits cross-reactivity with one or more other TREM2 proteins of another species. In some embodiments, a molecule that specifically binds to human TREM2 protein exhibits cross-reactivity with a cynomolgus monkey (“cyno”) TREM2 protein. In some embodiments, a molecule that specifically binds to human TREM2 protein exhibits cross-reactivity with a mouse TREM2 protein. In some embodiments, a molecule as described herein exhibits cross-reactivity with human TREM2, cyno TREM2, and mouse TREM2.

Methods for analyzing binding affinity, binding kinetics, and cross-reactivity are known in the art. These methods include, but are not limited to, solid-phase binding assays (e.g., ELISA assay), immunoprecipitation, surface plasmon resonance (e.g., Biacore™ (GE Healthcare, Piscataway, NJ)), kinetic exclusion assays (e.g., KinExA®), flow cytometry, fluorescence-activated cell sorting (FACS), BioLayer interferometry (e.g., Octet™ (FortéBio, Inc., Menlo Park, CA)), and western blot analysis. In some embodiments, ELISA is used to determine binding affinity and/or cross-reactivity. Methods for performing ELISA assays are known in the art. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross-reactivity.

Epitopes Recognized by Monovalent Anti-TREM2 Molecules that Also Bind TfR

In some embodiments, a molecule as described herein recognizes an epitope of human TREM2 that is the same or substantially the same as the epitope recognized by a clone as described herein or competes for binding with the clone. As used herein, the term “substantially the same,” as used with reference to an epitope recognized by a clone as described herein, means that a monovalent anti-TREM2 binding molecule as described herein recognizes an epitope that is identical, within, or nearly identical to (e.g., has at least 90% sequence identity to, or has one, two, or three amino acid substitutions, e.g., conservative substitutions, relative to), or has substantial overlap with (e.g., at least 50%, 60%, 70%, 80%, 90%, or 95% overlap with) the epitope recognized by the clone as described herein.

In some embodiments, a monovalent anti-TREM2 binding molecule recognizes an epitope of human TREM2 that is the same or substantially the same as the epitope recognized by a clone, or competes for binding with a clone, selected from the group consisting of Clone CL0020306, Clone CL0020188, Clone CL0020307, Clone A, Clone B, Clone C, Clone D, and variants of the same.

In some embodiments, a monovalent anti-TREM2 binding molecule binds to human TREM2 at an epitope within the stalk region of TREM2. In some embodiments, a monovalent anti-TREM2 binding molecule recognizes an epitope of human TREM2 comprising, within, or consisting of residues 129-172 or residues 131-169 of SEQ ID NO:1. In some embodiments, a monovalent anti-TREM2 binding molecule recognizes an epitope of human TREM2 comprising, within, or consisting of residues 129-148 of SEQ ID NO:1 (e.g., 143-148 of SEQ ID NO:1). In some embodiments, a monovalent anti-TREM2 binding molecule is an agonist that activates TREM2/DAP12 signaling (e.g., by inducing phosphorylation of a kinase such as Syk) and binds to human TREM2 at an epitope within the stalk region of TREM2. In some embodiments, a monovalent anti-TREM2 binding molecule binds to human TREM2 at an epitope within the stalk region of TREM2 and inhibits cleavage of TREM2 by a protease (e.g., ADAM17).

### Certain Fc Polypeptide Modifications

As described herein, a monovalent anti-TREM2 binding molecule may comprise two Fc polypeptides, wherein one or both of which may each comprise independently selected modifications (e.g., mutations) or may be a wild-type Fc polypeptide, e.g., a human IgG1 Fc polypeptide. Non-limiting examples of mutations that can be introduced into one or both Fc polypeptides include, e.g., mutations to increase serum stability, to modulate effector function, to influence glycosylation, to reduce immunogenicity in humans, and/or to provide for knob and hole heterodimerization of the Fc polypeptides. Additionally, as described below, one or both of the Fc polypeptides may be modified to comprise a second binding region that specifically binds to a transferrin receptor (e.g., a human or cynomolgus TfR, such as may be expressed on a brain endothelial cell).

In some embodiments, the Fc polypeptides present in a molecule as disclosed herein include knob and hole mutations to promote heterodimer formation and hinder homodimer formation. Generally, the modifications introduce a protuberance (“knob”) at the interface of a first polypeptide and a corresponding cavity (“hole”) in the interface of a second polypeptide, such that the protuberance can be positioned in the cavity so as to promote heterodimer formation and thus hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). In some embodiments, such additional mutations are at a position in the Fc polypeptide that does not have a negative (e.g., inhibitory) effect on binding of a Fc polypeptide to a TfR.

In one illustrative embodiment of a knob and hole approach for dimerization, position 366 (numbered according to the EU numbering scheme) of one of the Fc polypeptides present in the proteins described herein comprises a tryptophan in place of a native threonine. The other Fc polypeptide in the dimer has a valine at position 407 (numbered according to the EU numbering scheme) in place of the native tyrosine. The other Fc polypeptide may further comprise a substitution in which the native threonine at position 366 (numbered according to the EU numbering scheme) is substituted with a serine and a native leucine at position 368 (numbered according to the EU numbering scheme) is substituted with an alanine. Thus, one of the Fc polypeptides of a monovalent anti-TREM2 binding molecule of the disclosure has the T366W knob mutation and the other Fc polypeptide has the Y407V mutation, which is typically accompanied by the T366S and L368A hole mutations.

In some embodiments, one or both Fc polypeptides may also be engineered to contain other modifications for heterodimerization, e.g., electrostatic engineering of contact residues within a CH3-CH3 interface that are naturally charged or hydrophobic patch modifications.

In some embodiments, one or both Fc polypeptides in a molecule as described herein may comprise modifications that reduce effector function, i.e., having a reduced ability to induce certain biological functions upon binding to an Fc receptor expressed on an effector cell that mediates the effector function. Examples of antibody effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, one Fc polypeptide may include one or more modifications that modulate effector function or both Fc polypeptides may each independently include one or more modifications that modulate effector function.

In some embodiments, one or both Fc polypeptides may comprise independently selected modifications that reduce or eliminate effector function. Illustrative Fc polypeptide mutations that reduce effector function include, but are not limited to, substitutions in a CH2 domain, e.g., at positions 234 and 235, according to the EU numbering scheme. For example, in some embodiments, one or both Fc polypeptides can comprise alanine residues at positions 234 and 235. Thus, one or both Fc polypeptides may have L234A and L235A (LALA) substitutions.

Additional Fc polypeptide mutations that modulate an effector function include, but are not limited to, the following: position 329 may have a mutation in which proline is substituted with a glycine, arginine, serine, or an amino acid residue large enough to destroy the Fc/Fcγ receptor interface that is formed between proline 329 of the Fc and tryptophan residues Trp 87 and Trp 110 of FcγRIII. Additional illustrative substitutions include S228P, E233P, L235E, N297A, N297D, N297G, and P331S, according to the EU numbering scheme. Multiple substitutions may also be present, e.g., L234A and L235A of a human IgG1 Fc region; L234A, L235A, and P329G of a human IgG1 Fc region (LALAPG); L234A, L235A, and P329S of a human IgG1 Fc region (LALAPS); L234A, L235A, N297G, and P329G of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, according to the EU numbering scheme. In some embodiments, one or both Fc polypeptides may have one or more amino acid substitutions that modulate ADCC, e.g., substitutions at positions 298, 333, and/or 334, according to the EU numbering scheme.

In certain aspects, one or both of the Fc polypeptides (e.g., modified Fc polypeptides) present in a molecule as described herein, can comprise an FcRn binding site. In some embodiments, the FcRn binding site is within the Fc polypeptide or a fragment thereof.

In some embodiments, the FcRn binding site comprises a native FcRn binding site. In some embodiments, the FcRn binding site does not comprise amino acid changes relative to the amino acid sequence of a native FcRn binding site. In some embodiments, the native FcRn binding site is an IgG binding site, e.g., a human IgG binding site. In some embodiments, the FcRn binding site comprises a modification that alters FcRn binding.

In some embodiments, an FcRn binding site has one or more amino acid residues that are mutated, e.g., substituted, wherein the mutation(s) increase serum half-life or do not substantially reduce serum half-life (i.e., reduce serum half-life by no more than 25% compared to a counterpart Fc polypeptide having the wild-type residues at the mutated positions when assayed under the same conditions). In some embodiments, an FcRn binding site has one or more amino acid residues that are substituted at positions 251-256, 428, and 433-436, according to the EU numbering scheme.

In some embodiments, one or more residues at or near an FcRn binding site are mutated, relative to a native human IgG sequence, to extend serum half-life of the polypeptide.

In some embodiments, the mutations are M428L and/or N434S. In some embodiments, an Fc polypeptide further comprises the mutation N434S with or without M428L. In some embodiments, an Fc polypeptide comprises a mutation at one, two, or all three of positions T307, E380, and N434, according to the EU numbering scheme. In some embodiments, the mutations are T307Q and N434A. In some embodiments, an Fc polypeptide comprises mutations T307A, E380A, and N434A. In some embodiments, an Fc polypeptide comprises mutations at positions T250 and M428, according to the EU numbering scheme. In some embodiments, the Fc polypeptide comprises mutations T250Q and/or M428L. In some embodiments, an Fc polypeptide comprises mutations at positions M428 and N434, according to the EU numbering scheme. In some embodiments, the Fc polypeptide comprises mutations M428L and N434S (LS). In some embodiments, a molecule as described herein can comprise two Fc polypeptides, wherein each of the two Fc polypeptides comprises M428L and/or N434S substitutions. In some embodiments, the Fc polypeptide comprises an N434S or N434A mutation. In some embodiments, a molecule as described herein can comprise two Fc polypeptides, wherein each of the two Fc polypeptides comprises an N434S or N434A substitution.

By way of a non-limiting example, one or both of the Fc polypeptide sequences may independently comprise one or more modifications selected from the group consisting of a knob mutation (e.g., T366W as numbered according to the EU numbering scheme), hole mutations (e.g., T366S, L368A, and Y407V as numbered according to the EU numbering scheme), L234A, L235A, R292C, N297G, V302C, P329G, P331S, D356E, L358M, M428L, E430G, and N434S.

In some embodiments, the C-terminal Lys residue is removed in an Fc polypeptide described herein (i.e., the Lys residue at position 447, according to the EU numbering scheme).

As described herein, one or both of the Fc polypeptides may also further comprise a partial or full hinge region at the N-terminal region of the Fc polypeptide (e.g., a portion of an IgG1 hinge region, such as DKTHTCPPCP (SEQ ID NO:57). For example, in certain embodiments, the N-terminus of the second Fc polypeptide may include a portion of an IgG1 hinge region, such as DKTHTCPPCP (SEQ ID NO:57). In some embodiments, a Fc polypeptide comprises a sequence having at least 90%, 95%, 96%, 97%, 98% or 99% identity to an amino acid sequence set forth in any one of SEQ ID NOS:85, 86, 88, 97, 98, 100, 141, and 164. In some embodiments, a Fc polypeptide comprises or consists of the amino acid sequence set forth in any one of SEQ ID NOS:85, 86, 88, and 141. In some embodiments, a Fc polypeptide comprises or consists of the amino acid sequence set forth in any one of SEQ ID NOS:97, 98, 100, and 164.

Second Binding Region that Specifically Binds a TfR

As described herein, a molecule that is monovalent for specifically binding to TREM2 also comprises a second binding domain for specifically binding to a transferrin receptor. This second binding region may be comprised within a Fc polypeptide that is also present in the molecule (e.g., the Fc polypeptide comprises mutations that permit binding to a TfR) or may be a separate binding sequence (e.g., a sequence that is linked to the N-terminus or C-terminus of a Fc polypeptide described herein). In certain embodiments, a molecule as described herein may comprise more than one TfR binding region. In other embodiments, a molecule as described herein may be monovalent for binding to TfR (i.e., the molecule comprises a single second binding region).

Briefly, binding to a TfR protein (e.g., to the apical domain thereof) that is expressed on, for example, a brain endothelial cell, can, in some embodiments, permit a modified Fc polypeptide of this disclosure or a molecule comprising the same to cross the blood-brain barrier via receptor-mediated transcytosis. In certain embodiments, receptor-mediated transcytosis can enhance or improve the ability of the protein comprising the Fc polypeptide to be present in the brain (i.e., on the luminal side of the blood-brain barrier), which can allow for improved binding to TREM2 in the CNS, and other functions, e.g., clearance, neutralization, or immunodepletion of the target, or the like. Additionally, in certain embodiments, TfR binding in combination with TREM2 binding may result in enhanced modulation of TREM2 activity.

Examples of binding regions that specifically bind to a transferrin receptor are included below.

### Fc Modifications for Transferrin Receptor Binding

In some embodiments, a monovalent anti-TREM2 molecule that binds TfR includes an Fc polypeptide that comprises modifications (e.g., amino acid substitutions) that permit binding of the Fc polypeptide to a TfR protein (i.e., the Fc polypeptide has been modified to comprise a second binding region as described herein).

Exemplary TfR-binding amino acid modifications to an Fc (e.g., CH2 and/or CH3 portion, fragment, or domain), and Fc polypeptides and portions thereof that comprise the amino acid modifications, are described in PCT patent publication No. WO 2018/152326A1. These amino acid modifications, TfR-binding Fc polypeptide sequences and TfR-binding Fc polypeptides, and techniques for generating and testing the same are incorporated herein by reference. One or two Fc polypeptides of an Fc dimer of the present disclosure can be engineered to comprise modifications to permit binding to TfR. In certain embodiments, one Fc polypeptide of an Fc dimer comprises modifications to permit binding to TfR, and the other Fc polypeptide does not. In certain embodiments, the first Fc polypeptide is modified to comprise the second binding region.

In some embodiments, a Fc polypeptide modified to bind to a TfR comprises a YxTEWSS (SEQ ID NO:58) motif. In some embodiments, a Fc polypeptide modified to bind to a TfR comprises a TxxExxxxF (SEQ ID NO:59) motif. In some embodiments, a Fc polypeptide modified to bind to a TfR comprises a YxTEWSS (SEQ ID NO:58) and a TxxExxxxF (SEQ ID NO:59) motif.

In some embodiments, a Fc polypeptide modified to bind to a TfR comprises a wild-type amino acid residue at positions 380, 389, 390, and 415, according to EU numbering, wherein the wild-type amino acid residue is found at a corresponding position in SEQ ID NO:38.

In some embodiments, a molecule as described herein includes an Fc polypeptide having the following amino acids: Trp, Leu, or Glu at position 380; Tyr or Phe at position 384; Thr at position 386; Glu at position 387; Trp at position 388; Ser, Ala, or Val at position 389; Ser or Asn at position 390; Thr or Ser at position 413; Glu or Ser at position 415; Glu at position 416; and Phe at position 421, according to EU numbering.

In some embodiments, a molecule as described herein includes an Fc polypeptide that 1) comprises a sequence that has at least 80%, 85%, 90%, or 95% sequence identity to a sequence selected from the group consisting of SEQ ID NOS:38, 39, 46, 47, 49, 50, 61, 63, 68, 70, 84, 87, 93, 95, 99, 105, 107, 140, 143, 146, 147, 160-163, 166, 169, and 170; and 2) comprises: Trp, Leu, or Glu at position 380; Tyr or Phe at position 384; Thr at position 386; Glu at position 387; Trp at position 388; Ser, Ala, or Val at position 389; Ser or Asn at position 390; Thr or Ser at position 413; Glu or Ser at position 415; Glu at position 416; and Phe at position 421, according to EU numbering.

In some embodiments, a molecule as described herein includes an Fc polypeptide that 1) comprises a sequence that has at least 80%, 85%, 90% or 95% sequence identity to a sequence selected from the group consisting of SEQ ID NOS: 46, 47, 49, 50, 68, 70, 93, 95, 105, 107, 146, 147, 161, 162, 169, and 170; and 2) comprises: Trp, Leu, or Glu at position 380; Tyr or Phe at position 384; Thr at position 386; Glu at position 387; Trp at position 388; Ser, Ala, or Val at position 389; Ser or Asn at position 390; Thr or Ser at position 413; Glu or Ser at position 415; Glu at position 416; and Phe at position 421, according to EU numbering.

In some embodiments, a molecule as described herein includes an Fc polypeptide that 1) comprises a sequence that has at least 80%, 85%, 90% or 95% sequence identity to a sequence selected from the group consisting of SEQ ID NOS: 47, 50, 68, 70, 93, 95, 105, 107, 146, 147, 169, and 170; and 2) comprises: Trp, Leu, or Glu at position 380; Tyr or Phe at position 384; Thr at position 386; Glu at position 387; Trp at position 388; Ser, Ala, or Val at position 389; Ser or Asn at position 390; Thr or Ser at position 413; Glu or Ser at position 415; Glu at position 416; and Phe at position 421, according to EU numbering.

In some embodiments, the Fc polypeptide has the following amino acids: Trp at position 380; Tyr at position 384; Thr at position 386; Glu at position 387; Trp at position 388; Ser at position 389; Ser at position 390; Thr at position 413; Glu at position 415; Glu at position 416; and Phe at position 421, according to EU numbering.

In some embodiments, the Fc polypeptide has the following amino acids: Glu at position 380; Phe at position 384; Thr at position 386; Glu at position 387; Trp at position 388; Ser at position 389; Asn at position 390; Ser at position 413; Glu at position 415; Glu at position 416; and Phe at position 421, according to EU numbering.

In some embodiments, the Fc polypeptide has the following amino acids: Glu at position 380; Tyr at position 384; Thr at position 386; Glu at position 387; Trp at position 388; Val at position 389; Asn at position 390; Thr at position 413; Glu at position 415; Glu at position 416; and Phe at position 421, according to EU numbering.

In some embodiments, the Fc polypeptide has the following amino acids: Glu at position 380; Tyr at position 384; Thr at position 386; Glu at position 387; Trp at position 388; Ser at position 389; Asn at position 390; Ser at position 413; Glu at position 415; Glu at position 416; and Phe at position 421, according to EU numbering.

In some embodiments, the Fc polypeptide comprises a sequence having at least 90%, 95%, 96%, 97%, 98% or 99% identity to an amino acid sequence set forth in any one of SEQ ID NOS: 46, 47, 49, 50, 68, 70, 93, 95, 105, 107, 146, 147, 161, 162, 169, and 170. In some embodiments, the Fc polypeptide comprises a sequence having at least 90%, 95%, 96%, 97%, 98% or 99% identity to an amino acid sequence set forth in any one of SEQ ID NOS: 47, 50, 68, 70, 93, 95, 105, 107, 146, 147, 169, and 170. In some embodiments, the Fc polypeptide comprises or consists of the amino acid sequence set forth in any one of SEQ ID NOS: 47, 50, 68, 70, 93, 95, 105, 107, 146, 147, 169, and 170.

Additional examples of Fc polypeptides modified to bind to TfR are described in Table 8.

In some embodiments, a molecule as described herein comprises a first and a second Fc polypeptide, wherein the first polypeptide is modified to comprise the second binding region (i.e., the first polypeptide is modified to specifically bind to a TfR as described herein). In certain embodiments, the second Fc polypeptide is not modified to specifically bind to TfR. Thus, in certain embodiments, the first Fc polypeptide comprises a second binding region as described herein and the second Fc polypeptide comprises a sequence that has at least 90%, 95%, 96%, 97%, 98% or 99% sequence identity to a sequence selected from the group consisting of SEQ ID NOS: 38, 39, 61, 63, 84, 87, 99, 140, 143, 160, 163, and 166. In certain embodiments, the first Fc polypeptide comprises a second binding region as described herein and the second Fc polypeptide comprises a sequence that has at least 90%, 95%, 96%, 97%, 98% or 99% sequence identity to a sequence selected from the group consisting of SEQ ID NOS: 39, 61, 63, 84, 87, 99, 140, and 163. In some embodiments, the first Fc polypeptide comprises a second binding region as described herein and the second Fc polypeptide comprises or consists of the amino acid sequence set forth in any one of SEQ ID NOS: 39, 61, 63, 84, 87, 99, 140, and 163.

### Alternative Transferrin Receptor Binding Regions

In certain embodiments, rather than modifying a Fc polypeptide to specifically bind to a TfR, the second binding region comprises an antibody variable region sequence(s), which enables binding to TfR. For example, in certain embodiments, a molecule as described herein comprises a first and a second Fc polypeptide and the second binding region is linked to the first or the second Fc polypeptide. In certain embodiments, the second binding region is linked to the N-terminus of the first or second Fc polypeptide. In certain embodiments, the second binding region is linked to the C-terminus of the first or second Fc polypeptide. In certain embodiments, the first binding region is linked to the N-terminus of the first Fc polypeptide and the second binding region is linked to the C-terminus of the first Fc polypeptide.

In certain embodiments, the second binding region comprises an antibody variable region sequence(s). Anti-TfR antibodies are known in the art and variable region sequences from such antibodies may be included in a second binding region (i.e., for specific binding to TfR). In one example, a binding region comprising exemplary anti-TfR variable region sequences is described below and are included in Table 8.

In some embodiments, a second binding region comprises one or more complementarity determining region (CDR) sequences, a heavy chain variable region sequence, and/or a light chain variable region sequence as disclosed herein.

In some embodiments, the second binding region comprises one or more CDRs selected from the group consisting of:
(a) a heavy chain CDR1 (CDR-H1) comprising a sequence that has at least 90% sequence identity to the amino acid sequence TVSGFSLSSYAMS (SEQ ID NO:157), or that has up to two amino acid substitutions relative to the amino acid sequence of TVSGFSLSSYAMS (SEQ ID NO:157);
(b) a heavy chain CDR2 (CDR-H2) comprising a sequence that has at least 90% sequence identity to the amino acid sequence of YIWSGGSTD (SEQ ID NO:158), or that has up to two amino acid substitutions relative to the amino acid sequence of YIWSGGSTD (SEQ ID NO:158);
(c) a heavy chain CDR3 (CDR-H3) comprising a sequence that has at least 90% sequence identity to the amino acid sequence of ARRYGTSYPDYGDAQGFDP (SEQ ID NO:159), or that has up to two amino acid substitutions relative to the amino acid sequence of

(SEQ ID NO: 159)
ARRYGTSYPDYGDAQGFDP;

(d) a light chain CDR1 (CDR-L1) comprising a sequence that has at least 90% sequence identity to the amino acid sequence of RASQSISSYLA (SEQ ID NO:152), or that has up to two amino acid substitutions relative to the amino acid sequence of RASQSISSYLA (SEQ ID NO:152);
(e) a light chain CDR2 (CDR-L2) comprising a sequence that has at least 90% sequence identity to the amino acid sequence of YRASTLAS (SEQ ID NO:153), or that has up to two amino acid substitutions relative to the amino acid sequence of YRASTLAS (SEQ ID NO:153); and
(f) a light chain CDR3 (CDR-L3) comprising a sequence that has at least 90% sequence identity to the amino acid sequence of QQNYASSNVDNT (SEQ ID NO: 154), or that has up to two amino acid substitutions relative to the amino acid sequence of QQNYASSNVDNT (SEQ ID NO:154).

In some embodiments, the second binding region comprises two, three, four, five, or all six of (a)-(f). In some embodiments, the second binding region comprises the CDR-H1 of (a), the CDR-H2 of (b), and the CDR-H3 of (c). In some embodiments, the second binding region comprises the CDR-L1 of (d), the CDR-L2 of (e), and the CDR-L3 of (f). In some embodiments, a CDR having up to two amino acid substitutions has one amino acid substitution relative to the reference sequence. In some embodiments, a CDR having up to two amino acid substitutions has two amino acid substitutions relative to the reference sequence. In some embodiments, the up to two amino acid substitutions are conservative substitutions.

In some embodiments, the second binding region comprises one or more CDRs selected from the group consisting of:
(a) a CDR-H1 sequence comprising the amino acid sequence of SEQ ID NO:157;
(b) a CDR-H2 sequence comprising the amino acid sequence of SEQ ID NO:158;
(c) a CDR-H3 sequence comprising the amino acid sequence of SEQ ID NO:159;
(d) a CDR-L1 sequence comprising the amino acid sequence of SEQ ID NO: 152;
(e) a CDR-L2 sequence comprising the amino acid sequence of SEQ ID NO:153; and
(f) a CDR-L3 sequence comprising the amino acid sequence of SEQ ID NO:154.

In some embodiments, the second binding region comprises two, three, four, five, or all six of (a)-(f). In some embodiments, the second binding region comprises the CDR-H1 of (a), the CDR-H2 of (b), and the CDR-H3 of (c). In some embodiments, the second binding region comprises the CDR-L1 of (d), the CDR-L2 of (e), and the CDR-L3 of (f).

In some embodiments, the second binding region comprises a CDR-H1 comprising the amino acid sequence of SEQ ID NO:157, a CDR-H2 comprising the amino acid sequence of SEQ ID NO:158, a CDR-H3 comprising the amino acid sequence of SEQ ID NO:159, a CDR-L1 comprising the amino acid sequence of SEQ ID NO:152, a CDR-L2 comprising the amino acid sequence of SEQ ID NO:153, and a CDR-L3 comprising the amino acid sequence of SEQ ID NO:154.

In some embodiments, the second binding region comprises a heavy chain variable region comprising an amino acid sequence that has at least 85%, 90%, 91%, 92%, 93%, 94%, 95%, 96%, 97%, 98%, or 99% sequence identity to SEQ ID NO:156. In some embodiments, the second binding region comprises a heavy chain variable region comprising the amino acid sequence of SEQ ID NO:156.

In some embodiments, the second binding region comprises a light chain variable region comprising an amino acid sequence that has at least 85%, 90%, 91%, 92%, 93%, 94%, 95%, 96%, 97%, 98%, or 99% sequence identity to SEQ ID NO:151. In some embodiments, the second binding region comprises a light chain variable region comprising the amino acid sequence of SEQ ID NO:151.

In some embodiments, the second binding region comprises: a heavy chain variable region comprising an amino acid sequence that has at least 85%, 90%, 91%, 92%, 93%, 94%, 95%, 96%, 97%, 98%, or 99% sequence identity to SEQ ID NO:156, and a light chain variable region comprising an amino acid sequence that has at least 85%, 90%, 91%, 92%, 93%, 94%, 95%, 96%, 97%, 98%, or 99% sequence identity to SEQ ID NO:151. In some embodiments, the second binding region comprises: a heavy chain variable region comprising the amino acid sequence of SEQ ID NO:156, and a light chain variable region comprising the amino acid sequence of SEQ ID NO:151.

In some embodiments, the second binding region comprises a heavy chain variable region that comprises a heavy chain CDR1-3 comprising the amino acid sequences of SEQ ID NOS:157, 158, and 159, respectively, and that has at least 85% sequence identity (e.g., at least 90%, 91%, 92%, 93%, 94%, 95%, 96%, 97%, 98%, or 99% sequence identity) to SEQ ID NO:156. In some embodiments, the second binding region comprises a light chain variable region that comprises a light chain CDR1-3 comprising the amino acid sequences of SEQ ID NOS:152, 153, and 154, respectively, and that has at least 85% sequence identity (e.g., at least 90%, 91%, 92%, 93%, 94%, 95%, 96%, 97%, 98%, or 99% sequence identity) to SEQ ID NO:151.

In certain embodiments, the second binding region comprises a scFab. Thus, in some embodiments, the second binding region, comprises from N-terminus to C-terminus: a) a light chain comprising or consisting of an amino acid sequence that has at least 85%, 90%, 91%, 92%, 93%, 94%, 95%, 96%, 97%, 98%, 99% or 100% sequence identity to SEQ ID NO: 150; b) a polypeptide linker; and c) a Fd region comprising or consisting of an amino acid sequence that has at least 85%, 90%, 91%, 92%, 93%, 94%, 95%, 96%, 97%, 98%, 99% or 100% sequence identity to SEQ ID NO: 155. In some embodiments, the second binding region, comprises from N-terminus to C-terminus: a) a light chain comprising or consisting of an amino acid sequence set forth in SEQ ID NO: 150; b) a polypeptide linker; and c) a Fd region comprising or consisting of the amino acid sequence set forth in SEQ ID NO: 155.

In some embodiments, the light chain of the second binding region is linked to an Fd region (e.g., the C-terminus of the light chain is linked to the N-terminus of the Fd region) by a linker, e.g., a peptide linker. The peptide linker may be configured such that it allows for the proper orientation of the variable region sequences to facilitate binding to a TfR. Peptide linkers may contain natural amino acids, unnatural amino acids, or a combination thereof. In some embodiments, the peptide linker may be a flexible linker, e.g., containing amino acids such as Gly, Asn, Ser, Thr, Ala, and the like (e.g., a glycine-rich linker). Such linkers are designed using known parameters and may contain, e.g., a number of repeat units (e.g., repeat units of Gly and Ser residues). For example, the linker may have repeats, such as two, three, four, five, six, or more Gly₄-Ser (SEQ ID NO:172) repeats (see, e.g., SEQ ID NO:173). In certain aspects, the linker may be GGGGSGGGGSGGGGSGGGGSGGGGSGGGGSGG (SEQ ID NO:149).

### IV. Certain Embodiments of Monovalent Anti-TREM2 Binding Molecules that Bind to a Transferrin Receptor

As described herein, certain embodiments provide a molecule comprising:
(a) a first binding region that specifically binds to TREM2; and
(b) a second binding region that specifically binds to a transferrin receptor;

wherein the molecule is monovalent for binding to TREM2. In certain embodiments, the molecule further comprises a first and a second Fc polypeptide, wherein the second Fc polypeptide forms a dimer with the first Fc polypeptide.

Variable regions for TREM2 and TfR binding, as well as Fc modifications for binding TfR are described above and in Table 8. Certain exemplary combinations of these sequences are included below.

### Embodiments of Molecules Comprising a Modified TfR-Binding Fc Polypeptide

In certain embodiments, an Fc polypeptide present within a molecule described herein is modified to comprise the second binding region (i.e., is modified to bind to a TfR). For example, in certain embodiments, the first Fc polypeptide comprises a sequence having at least 85%, 90%, or 95% identity to an amino acid sequence selected from the group consisting of SEQ ID NOS: 47, 93, 68, and 105, and the second Fc polypeptide comprises a sequence having at least 85%, 90%, or 95% identity to an amino acid sequence selected from the group consisting of SEQ ID NOS: 39, 87, 63, 99, 61 and 84. In certain embodiments, the first Fc polypeptide comprises a sequence selected from the group consisting of SEQ ID NOS:47, 93, 68, and 105, and the second Fc polypeptide comprises a sequence selected from the group consisting of SEQ ID NOS: 39, 87, 63, 99, 61 and 84.

In certain embodiments, the molecule comprises:
(i) a heavy chain (HC) comprising 1) a V_{H} comprising a sequence that has at least 85%, 90%, 95% or 100% identity to any one of SEQ ID NOS:24, 113, 121, 129, and 136; and 2) the first Fc polypeptide comprising a sequence having at least 85%, 90%, 95% or 100% identity to any one of SEQ ID NOS:47, 93, 68, and 105 (e.g., 100% identity to SEQ ID NO:47);
(ii) the second Fc polypeptide comprising a sequence having at least 85%, 90%, 95% or 100% identity to any one of SEQ ID NOS:39, 87, 63, 99, 61 and 84 (e.g., 100% identity to SEQ ID NO:39); and
(iii) a light chain comprising a V_{L} comprising a sequence that has at least 85%, 90%, 95% or 100% identity to any one of SEQ ID NOS:22, 109, 117, 125, and 132.

In certain embodiments, the molecule comprises:
(i) a heavy chain (HC) comprising 1) a V_{H} comprising a sequence selected from the group consisting of SEQ ID NOS:24, 113, 121, 129, and 136; and 2) the first Fc polypeptide comprising a sequence having at least 85%, 90%, 95% or 100% identity to SEQ ID NO:47 (e.g., 100% identity to SEQ ID NO:47);
(ii) the second Fc polypeptide comprising a sequence having at least 85%, 90%, 95% or 100% identity to SEQ ID NO:39 (e.g., 100% identity to SEQ ID NO:39); and
(iii) a light chain comprising a V_{L} comprising a sequence selected from the group consisting of SEQ ID NOS:22, 109, 117, 125, and 132.

In certain embodiments, the molecule comprises:
(i) a heavy chain (HC) comprising 1) a V_{H} comprising a sequence selected from the group consisting of SEQ ID NOS:24, 113, 121, 129, and 136; and 2) the first Fc polypeptide comprising a sequence having at least 85%, 90%, 95% or 100% identity to SEQ ID NO:47 (e.g., 100% identity to SEQ ID NO:47);
(ii) the second Fc polypeptide comprising a sequence having at least 85%, 90%, 95% or 100% identity to SEQ ID NO:61 (e.g., 100% identity to SEQ ID NO:61); and
(iii) a light chain comprising a V_{L} comprising a sequence selected from the group consisting of SEQ ID NOS:22, 109, 117, 125, and 132.

In certain embodiments, the molecule comprises:
(i) a heavy chain (HC) comprising 1) a V_{H} comprising a sequence selected from the group consisting of SEQ ID NOS:24, 113, 121, 129, and 136; and 2) the first Fc polypeptide comprising a sequence having at least 85%, 90%, 95% or 100% identity to SEQ ID NO:68 (e.g., 100% identity to SEQ ID NO:68);
(ii) the second Fc polypeptide comprising a sequence having at least 85%, 90%, 95% or 100% identity to SEQ ID NO:63 (e.g., 100% identity to SEQ ID NO:63); and
(iii) a light chain comprising a V_{L} comprising a sequence selected from the group consisting of SEQ ID NOS:22, 109, 117, 125, and 132.

In certain embodiments, the molecule comprises:
(i) a heavy chain (HC) comprising 1) a V_{H} comprising a sequence selected from the group consisting of SEQ ID NOS:24, 113, 121, 129, and 136; and 2) the first Fc polypeptide comprising a sequence having at least 85%, 90%, 95% or 100% identity to SEQ ID NO:68 (e.g., 100% identity to SEQ ID NO:68);
(ii) the second Fc polypeptide comprising a sequence having at least 85%, 90%, 95% or 100% identity to SEQ ID NO:84 (e.g., 100% identity to SEQ ID NO:84); and
(iii) a light chain comprising a V_{L} comprising a sequence selected from the group consisting of SEQ ID NOS:22, 109, 117, 125, and 132.

In certain embodiments, the molecule comprises:
(i) a heavy chain (HC) comprising 1) a V_{H} comprising a sequence selected from the group consisting of SEQ ID NOS:24, 113, 121, 129, and 136; and 2) the first Fc polypeptide comprising a sequence having at least 85%, 90%, 95% or 100% identity to SEQ ID NO:93 (e.g., 100% identity to SEQ ID NO:93);
(ii) the second Fc polypeptide comprising a sequence having at least 85%, 90%, 95% or 100% identity to SEQ ID NO:87 (e.g., 100% identity to SEQ ID NO:87); and
(iii) a light chain comprising a V_{L} comprising a sequence selected from the group consisting of SEQ ID NOS:22, 109, 117, 125, and 132.

In certain embodiments, the molecule comprises:
(i) a heavy chain (HC) comprising 1) a V_{H} comprising a sequence selected from the group consisting of SEQ ID NOS:24, 113, 121, 129, and 136; and 2) the first Fc polypeptide comprising a sequence having at least 85%, 90%, 95% or 100% identity to SEQ ID NO: 105 (e.g., 100% identity to SEQ ID NO:105);
(ii) the second Fc polypeptide comprising a sequence having at least 85%, 90%, 95% or 100% identity to SEQ ID NO:99 (e.g., 100% identity to SEQ ID NO:99); and
(iii) a light chain comprising a V_{L} comprising a sequence selected from the group consisting of SEQ ID NOS:22, 109, 117, 125, and 132.

In certain embodiments:
(a) the V_{H} comprises SEQ ID NO:24 and the V_{L} comprises SEQ ID NO:22;
(b) the V_{H} comprises SEQ ID NO:113 and the V_{L} comprises SEQ ID NO: 109;
(c) the V_{H} comprises SEQ ID NO:121 and the V_{L} comprises SEQ ID NO:117;
(d) the V_{H} comprises SEQ ID NO:129 and the V_{L} comprises SEQ ID NO:125; or
(e) the V_{H} comprises SEQ ID NO:136 and the V_{L} comprises SEQ ID NO:132.

In certain embodiments, the molecule comprises:
(i) a heavy chain (HC) that comprises a sequence having at least 90%, 95% or 100% identity to any one of SEQ ID NOS:48, 69, 94, 106;
(ii) the second Fc polypeptide that comprises a sequence having at least 90%, 95% or 100% identity to any one of SEQ ID NOS:85, 86, 88, 97, 98, and 100; and
(iii) a light chain (LC) that comprises a sequence having at least 90%, 95% or 100% identity to SEQ ID NO:54.

In certain embodiments, the molecule comprises:
(i) a heavy chain (HC) that comprises or consists of the amino acid sequence set forth in SEQ ID NO:48;
(ii) the second Fc polypeptide that comprises or consists of the amino acid sequence set forth in SEQ ID NO:86; and
(iii) a light chain (LC) that comprises or consists of the amino acid sequence set forth in SEQ ID NO:54.

In certain embodiments, the molecule comprises:
(i) a heavy chain (HC) that comprises or consists of the amino acid sequence set forth in SEQ ID NO:48;
(ii) the second Fc polypeptide that comprises or consists of the amino acid sequence set forth in SEQ ID NO:85; and
(iii) a light chain (LC) that comprises or consists of the amino acid sequence set forth in SEQ ID NO:54.

In certain embodiments, the molecule comprises:
(i) a heavy chain (HC) that comprises or consists of the amino acid sequence set forth in SEQ ID NO:69;
(ii) the second Fc polypeptide that comprises or consists of the amino acid sequence set forth in SEQ ID NO:98; and
(iii) a light chain (LC) that comprises or consists of the amino acid sequence set forth in SEQ ID NO:54.

In certain embodiments, the molecule comprises:
(i) a heavy chain (HC) that comprises or consists of the amino acid sequence set forth in SEQ ID NO:69;
(ii) the second Fc polypeptide that comprises or consists of the amino acid sequence set forth in SEQ ID NO:97; and
(iii) a light chain (LC) that comprises or consists of the amino acid sequence set forth in SEQ ID NO:54.

In certain embodiments, the molecule comprises:
(i) a heavy chain (HC) that comprises or consists of the amino acid sequence set forth in SEQ ID NO:94;
(ii) the second Fc polypeptide that comprises or consists of the amino acid sequence set forth in SEQ ID NO:88; and
(iii) a light chain (LC) that comprises or consists of the amino acid sequence set forth in SEQ ID NO:54.

In certain embodiments, the molecule comprises:
(i) a heavy chain (HC) that comprises or consists of the amino acid sequence set forth in SEQ ID NO:106;
(ii) the second Fc polypeptide that comprises or consists of the amino acid sequence set forth in SEQ ID NO:100; and
(iii) a light chain (LC) that comprises or consists of the amino acid sequence set forth in SEQ ID NO:54.

In certain embodiments the first Fc polypeptide comprises a sequence having at least 85%, 90%, or 95% identity to an amino acid sequence selected from the group consisting of SEQ ID NOS: 50, 95, 70, and 107, and the second Fc polypeptide comprises a sequence having at least 85%, 90%, or 95% identity to an amino acid sequence selected from the group consisting of SEQ ID NOS: 39, 87, 63, and 99. In certain embodiments, the first Fc polypeptide comprises a sequence selected from the group consisting of SEQ ID NOS:50, 95, 70, and 107, and the second Fc polypeptide comprises a sequence selected from the group consisting of SEQ ID NO:39, 87, 63, and 99.

In certain embodiments, the molecule comprises:
(i) a heavy chain (HC) comprising 1) a V_{H} comprising a sequence that has at least 85%, 90%, 95% or 100% identity to any one of SEQ ID NOS:24, 113, 121, 129, and 136; and 2) the first Fc polypeptide comprising a sequence having at least 85%, 90%, 95% or 100% identity to any one of SEQ ID NOS: 50, 95, 70, and 107 (e.g., 100% identity to SEQ ID NO:50);
(ii) the second Fc polypeptide comprising a sequence having at least 85%, 90%, 95% or 100% identity to any one of SEQ ID NOS: 39, 87, 63, and 99 (e.g., 100% identity to SEQ ID NO:39); and
(iii) a light chain comprising a V_{L} comprising a sequence that has at least 85%, 90%, 95% or 100% identity to any one of SEQ ID NOS:22, 109, 117, 125, and 132.

In certain embodiments, the molecule comprises:
(i) a heavy chain (HC) comprising 1) a V_{H} comprising a sequence selected from the group consisting of SEQ ID NOS:24, 113, 121, 129, and 136; and 2) the first Fc polypeptide comprising a sequence having at least 85%, 90%, 95% or 100% identity to SEQ ID NO:50 (e.g., 100% identity to SEQ ID NO:50);
(ii) the second Fc polypeptide comprising a sequence having at least 85%, 90%, 95% or 100% identity to SEQ ID NO:39 (e.g., 100% identity to SEQ ID NO:39); and
(iii) a light chain comprising a V_{L} comprising a sequence selected from the group consisting of SEQ ID NOS:22, 109, 117, 125, and 132.

In certain embodiments, the molecule comprises:
(i) a heavy chain (HC) comprising 1) a V_{H} comprising a sequence selected from the group consisting of SEQ ID NOS:24, 113, 121, 129, and 136; and 2) the first Fc polypeptide comprising a sequence having at least 85%, 90%, 95% or 100% identity to SEQ ID NO:70 (e.g., 100% identity to SEQ ID NO:70);
(ii) the second Fc polypeptide comprising a sequence having at least 85%, 90%, 95% or 100% identity to SEQ ID NO:63 (e.g., 100% identity to SEQ ID NO:63); and
(iii) a light chain comprising a V_{L} comprising a sequence selected from the group consisting of SEQ ID NOS:22, 109, 117, 125, and 132.

In certain embodiments, the molecule comprises:
(i) a heavy chain (HC) comprising 1) a V_{H} comprising a sequence selected from the group consisting of SEQ ID NOS:24, 113, 121, 129, and 136; and 2) the first Fc polypeptide comprising a sequence having at least 85%, 90%, 95% or 100% identity to SEQ ID NO:95 (e.g., 100% identity to SEQ ID NO:95);
(ii) the second Fc polypeptide comprising a sequence having at least 85%, 90%, 95% or 100% identity to SEQ ID NO:87 (e.g., 100% identity to SEQ ID NO:87); and
(iii) a light chain comprising a V_{L} comprising a sequence selected from the group consisting of SEQ ID NOS:22, 109, 117, 125, and 132.

In certain embodiments, the molecule comprises:
(i) a heavy chain (HC) comprising 1) a V_{H} comprising a sequence selected from the group consisting of SEQ ID NOS:24, 113, 121, 129, and 136; and 2) the first Fc polypeptide comprising a sequence having at least 85%, 90%, 95% or 100% identity to SEQ ID NO: 107 (e.g., 100% identity to SEQ ID NO:107);
(ii) the second Fc polypeptide comprising a sequence having at least 85%, 90%, 95% or 100% identity to SEQ ID NO:99 (e.g., 100% identity to SEQ ID NO:99); and
(iii) a light chain comprising a V_{L} comprising a sequence selected from the group consisting of SEQ ID NOS:22, 109, 117, 125, and 132.

In certain embodiments:
(a) the V_{H} comprises SEQ ID NO:24 and the V_{L} comprises SEQ ID NO:22;
(b) the V_{H} comprises SEQ ID NO:113 and the V_{L} comprises SEQ ID NO: 109;
(c) the V_{H} comprises SEQ ID NO:121 and the V_{L} comprises SEQ ID NO: 117;
(d) the V_{H} comprises SEQ ID NO:129 and the V_{L} comprises SEQ ID NO:125; or
(e) the V_{H} comprises SEQ ID NO:136 and the V_{L} comprises SEQ ID NO:132.

In certain embodiments, the molecule comprises:
(i) a heavy chain (HC) that comprises a sequence having at least 90%, 95% or 100% identity to any one of SEQ ID NOS: 51, 71, 96, and 108;
(ii) the second Fc polypeptide that comprises a sequence having at least 90%, 95% or 100% identity to any one of SEQ ID NOS: 86, 88, 98, and 100; and
(iii) a light chain (LC) that comprises a sequence having at least 90%, 95% or 100% identity to SEQ ID NO:54.

In certain embodiments, the molecule comprises:
(i) a heavy chain (HC) that comprises or consists of the amino acid sequence set forth in SEQ ID NO:51;
(ii) the second Fc polypeptide that comprises or consists of the amino acid sequence set forth in SEQ ID NO:86; and
(iii) a light chain (LC) that comprises or consists of the amino acid sequence set forth in SEQ ID NO:54.

In certain embodiments, the molecule comprises:
(i) a heavy chain (HC) that comprises or consists of the amino acid sequence set forth in SEQ ID NO:71;
(ii) the second Fc polypeptide that comprises or consists of the amino acid sequence set forth in SEQ ID NO:98; and
(iii) a light chain (LC) that comprises or consists of the amino acid sequence set forth in SEQ ID NO:54.

In certain embodiments, the molecule comprises:
(i) a heavy chain (HC) that comprises or consists of the amino acid sequence set forth in SEQ ID NO:96;
(ii) the second Fc polypeptide that comprises or consists of the amino acid sequence set forth in SEQ ID NO:88; and
(iii) a light chain (LC) that comprises or consists of the amino acid sequence set forth in SEQ ID NO:54.

In certain embodiments, the molecule comprises:
(i) a heavy chain (HC) that comprises or consists of the amino acid sequence set forth in SEQ ID NO:108;
(ii) the second Fc polypeptide that comprises or consists of the amino acid sequence set forth in SEQ ID NO: 100; and
(iii) a light chain (LC) that comprises or consists of the amino acid sequence set forth in SEQ ID NO:54.

### Embodiments of Molecules Comprising Fc Polypeptides Linked a Second Binding Region

In certain other embodiments, a molecule described herein comprises a second binding region that is linked to the first and/or second Fc polypeptide comprised within the molecule. In certain embodiments, the first and/or second Fc polypeptide comprises a sequence that has at least 85%, 90% or 95% sequence identity to a sequence selected from the group consisting of SEQ ID NOS: 38, 140, 143, 160, 163, and 166. In certain embodiments, the first Fc polypeptide comprises a sequence that has at least 85%, 90% or 95% sequence identity to a sequence selected from the group consisting of 143 and 166. In certain embodiments, the second Fc polypeptide comprises a sequence that has at least 85%, 90% or 95% sequence identity to a sequence selected from the group consisting of 140 and 163. In certain embodiments, the first Fc polypeptide comprises a sequence that has at least 85%, 90% or 95% sequence identity to a sequence selected from the group consisting of SEQ ID NO:143 or 166, and the second Fc polypeptide comprises a sequence that has at least 85%, 90% or 95% sequence identity to a sequence selected from the group consisting of SEQ ID NO:140 or 163. In certain embodiments, the first Fc polypeptide comprises SEQ ID NO: 143 or 166, and the second Fc polypeptide comprises SEQ ID NO:140 or 163.

In certain embodiments, the molecule comprises:
(i) a heavy chain (HC) linked to the second binding region (SBR) comprising 1) a V_{H} comprising a sequence that has at least 85%, 90%, 95% or 100% identity to any one of SEQ ID NOS:24, 113, 121, 129, and 136; 2) the first Fc polypeptide comprising a sequence having at least 85%, 90%, 95% or 100% identity to SEQ ID NO: 143 or 166; and 3) the SBR comprising a SBR V_{H} comprising a sequence that has at least 85%, 90%, 95% or 100% identity to SEQ ID NO:156 and a SBR V_{L} comprising a sequence that has at least 85%, 90%, 95% or 100% identity to SEQ ID NO:151;
(ii) the second Fc polypeptide comprising a sequence having at least 85%, 90%, 95% or 100% identity to SEQ ID NO:140 or 163; and
(iii) a light chain comprising a V_{L} comprising a sequence that has at least 85%, 90%, 95% or 100% identity to any one of SEQ ID NOS:22, 109, 117, 125, and 132.

In certain embodiments, the molecule comprises:
(i) a heavy chain (HC) linked to the second binding region (SBR) comprising 1) a V_{H} comprising a sequence selected from the group consisting of SEQ ID NOS:24, 113, 121, 129, and 136; 2) the first Fc polypeptide comprising a sequence having at least 85%, 90%, 95% or 100% identity to SEQ ID NO:166 (e.g., 100% identity to SEQ ID NO:166); and 3) the SBR comprising a SBR V_{H} sequence comprising SEQ ID NO:156 and a SBR V_{L} sequence comprising SEQ ID NO:151;
(ii) the second Fc polypeptide comprising a sequence having at least 85%, 90%, 95% or 100% identity to SEQ ID NO:140 (e.g., 100% identity to SEQ ID NO:140); and
(iii) a light chain comprising a V_{L} comprising a sequence selected from the group consisting of SEQ ID NOS:22, 109, 117, 125, and 132.

In certain embodiments, the molecule comprises:
(i) a heavy chain (HC) linked to the second binding region (SBR) comprising 1) a V_{H} comprising a sequence selected from the group consisting of SEQ ID NOS:24, 113, 121, 129, and 136; 2) the first Fc polypeptide comprising a sequence having at least 85%, 90%, 95% or 100% identity to SEQ ID NO:166 (e.g., 100% identity to SEQ ID NO:166); and 3) the SBR comprising a SBR V_{H} sequence comprising SEQ ID NO:156 and a SBR V_{L} sequence comprising SEQ ID NO:151;
(ii) the second Fc polypeptide comprising a sequence having at least 85%, 90%, 95% or 100% identity to SEQ ID NO:163 (e.g., 100% identity to SEQ ID NO:163); and
(iii) a light chain comprising a V_{L} comprising a sequence selected from the group consisting of SEQ ID NOS:22, 109, 117, 125, and 132.

In certain embodiments:
(a) the V_{H} comprises SEQ ID NO:24 and the V_{L} comprises SEQ ID NO:22;
(b) the V_{H} comprises SEQ ID NO:113 and the V_{L} comprises SEQ ID NO:109;
(c) the V_{H} comprises SEQ ID NO:121 and the V_{L} comprises SEQ ID NO:117;
(d) the V_{H} comprises SEQ ID NO:129 and the V_{L} comprises SEQ ID NO: 125; or
(e) the V_{H} comprises SEQ ID NO:136 and the V_{L} comprises SEQ ID NO:132.

In certain embodiments, the molecule comprises:
(i) a heavy chain (HC) linked to a second binding region (SBR), which comprises from N-terminus to C-terminus: 1) the heavy chain comprising a sequence having at least 90%, 95% or 100% identity to SEQ ID NO: 168; 2) a first polypeptide linker; and 3) the SBR, which comprises from N-terminus to C-terminus: a) a SBR light chain comprising a sequence having at least 90%, 95% or 100% identity to SEQ ID NO: 150; b) a second polypeptide linker; and c) a Fd region comprising a sequence having at least 90%, 95% or 100% identity to any one of SEQ ID NO: 155;
(ii) the second Fc polypeptide that comprises a sequence having at least 90%, 95% or 100% identity to SEQ ID NO:141 or 164; and
(iii) a light chain (LC) that comprises a sequence having at least 90%, 95% or 100% identity to SEQ ID NO:54. In certain embodiments, the first and/or the second polypeptide linker are a linker described herein, such as a Gly- and/or Ser-rich linker.

In certain embodiments, the molecule comprises:
(i) a heavy chain (HC) linked to a second binding region (SBR), which comprises from N-terminus to C-terminus: 1) the heavy chain comprising or consisting of the amino acid sequence set forth in SEQ ID NO:168; 2) a first polypeptide linker; and 3) the SBR, which comprises from N-terminus to C-terminus: a) a SBR light chain comprising or consisting of the amino acid sequence set forth in SEQ ID NO: 150; b) a second polypeptide linker; and c) a Fd region comprising or consisting of the amino acid sequence set forth in SEQ ID NO: 155;
(ii) the second Fc polypeptide that comprises or consists of the amino acid sequence set forth in SEQ ID NO:141; and
(iii) a light chain (LC) that comprises or consists of the amino acid sequence set forth in SEQ ID NO:54. In certain embodiments, the first and/or the second polypeptide linker are a linker described herein, such as a Gly- and/or Ser-rich linker.

In certain embodiments, the molecule comprises:
(i) a heavy chain (HC) linked to a second binding region (SBR), which comprises from N-terminus to C-terminus: 1) the heavy chain comprising or consisting of the amino acid sequence set forth in SEQ ID NO: 168; 2) a first polypeptide linker; and 3) the second binding region, which comprises from N-terminus to C-terminus: a) a SBR light chain comprising or consisting of the amino acid sequence set forth in SEQ ID NO: 150; b) a second polypeptide linker; and c) a Fd region comprising or consisting of the amino acid sequence set forth in SEQ ID NO: 155;
(ii) the second Fc polypeptide that comprises or consists of the amino acid sequence set forth in SEQ ID NO: 164; and
(iii) a light chain (LC) that comprises or consists of the amino acid sequence set forth in SEQ ID NO:54. In certain embodiments, the first and/or the second polypeptide linker are a linker described herein, such as a Gly- and/or Ser-rich linker.

In certain embodiments, the molecule comprises:
(i) a heavy chain (HC) linked to the second binding region that comprises a sequence having at least 90%, 95% or 100% identity to SEQ ID NO:171;
(ii) the second Fc polypeptide that comprises a sequence having at least 90%, 95% or 100% identity to SEQ ID NO:141 or 164; and
(iii) a light chain (LC) that comprises a sequence having at least 90%, 95% or 100% identity to SEQ ID NO:54.

In certain embodiments, the molecule comprises:
(i) a heavy chain (HC) linked to the second binding region that comprises or consists of SEQ ID NO:171;
(ii) the second Fc polypeptide that comprises or consists of the amino acid sequence set forth in SEQ ID NO:141; and
(iii) a light chain (LC) that comprises or consists of the amino acid sequence set forth in SEQ ID NO:54.

In certain embodiments, the molecule comprises:
(i) a heavy chain (HC) linked to the second binding region that comprises or consists of SEQ ID NO:171;
(ii) the second Fc polypeptide that comprises or consists of the amino acid sequence set forth in SEQ ID NO: 164; and
(iii) a light chain (LC) that comprises or consists of the amino acid sequence set forth in SEQ ID NO:54.

### V. Certain Characteristics of Monovalent Anti-TREM2 Binding Molecules that Bind a TfR

As described herein, the combination of TREM2 and TfR specific binding in a single molecule enhances the modulating TREM2 capabilities of the molecule, enabling monovalent anti-TREM2 formats.

Accordingly, in certain embodiments, a monovalent anti-TREM2 molecule that binds TfR is capable of modulating (i.e., increasing or decreasing) TREM2 activity to a greater degree than a corresponding molecule that does not bind to a transferrin receptor.

The term “TREM2 activity” may be used to refer to a single function of the protein or to a combination of activities. For example, the TREM2 activity may be an activity known in the art or described herein, such as a TREM2 activity described below (e.g., pSyk signaling activity). A skilled artisan is capable of evaluating TREM2 activity using an assay known in the art or described herein.

As used herein, the term “corresponding molecule” refers to a molecule having the same structure and sequence as its counterpart, with the exception that the corresponding molecule does not comprise a second binding region that specifically binds to a transferrin receptor.

Comparison of the effects of a molecule of the disclosure and its counterpart on TREM2 activity also takes into consideration access to the protein; the difference in modulating activity does not simply result from differing access to the protein. Thus, the second binding region that specifically binds to a transferrin receptor not only may enhance delivery of the molecule (e.g., delivery across the BBB) but is also capable of enhancing the signaling capabilities of the molecule itself when contacted with TREM2.

In certain embodiments, the monovalent anti-TREM2 molecule that binds TfR is an agonist that increases or enhances TREM2 activity to a greater degree than a corresponding molecule that does not bind to a transferrin receptor. In certain embodiments, the molecule increases TREM2 activity by at least about 1.25-fold, 1.5-fold, 1.75-fold, 2-fold, 3-fold, 4-fold, 5-fold, 6-fold, 7-fold, 8-fold, 9-fold, 10-fold or more as compared the corresponding molecule.

In certain embodiments, a monovalent anti-TREM2 molecule that binds TfR is capable of modulating TREM2 activity (e.g., enhancing TREM2 activity) by at least the same degree as a corresponding molecule that is bivalent for TREM2 binding and that does not bind to a transferrin receptor.

In certain embodiments, a monovalent anti-TREM2 molecule that binds TfR has an increased avidity to TREM2 as compared to a corresponding molecule that does not bind to a transferrin receptor. In certain embodiments, the molecule's avidity to TREM2 is at least about 1.25-fold, 1.5-fold, 1.75-fold, 2-fold, 3-fold, 4-fold, 5-fold, 6-fold, 7-fold, 8-fold, 9-fold, 10-fold or more as compared to the corresponding molecule.

In certain embodiments, a monovalent anti-TREM2 molecule that binds TfR is capable of enhancing signaling of TREM2 on a cell surface as compared to a corresponding molecule that does not bind to a transferrin receptor. As used herein, the term “signaling” refers to an increase, e.g., in the phosphorylation of Syk, a signaling mediator of TREM2 and the co-receptor DAP12. In certain embodiments, the molecule increases signaling of TREM2 by at least about 1.25-fold, 1.5-fold, 1.75-fold, 2-fold, 3-fold, 4-fold, 5-fold, 6-fold, 7-fold, 8-fold, 9-fold, 10-fold or more as compared the corresponding molecule.

In certain embodiments, a monovalent anti-TREM2 molecule that binds TfR has enhanced cellular internalization and/or endosomal localization as compared to a corresponding molecule that does not bind to a transferrin receptor. In certain embodiments, internalization of the molecule is greater by at least about 1.25-fold, 1.5-fold, 1.75-fold, 2-fold, 3-fold, 4-fold, 5-fold, 6-fold, 7-fold, 8-fold, 9-fold, 10-fold or more as compared the corresponding molecule. In certain embodiments, endosomal localization of the molecule is greater by at least about 1.25-fold, 1.5-fold, 1.75-fold, 2-fold, 3-fold, 4-fold, 5-fold, 6-fold, 7-fold, 8-fold, 9-fold, 10-fold or more as compared the corresponding molecule. In certain embodiments, a monovalent anti-TREM2 molecule that binds TfR enhances endosomal signaling as compared to a corresponding molecule that does not bind to a transferrin receptor. In certain embodiments, the molecule increases endosomal signaling at least more than about 1.25-fold, 1.5-fold, 1.75-fold, 2-fold, 3-fold, 4-fold, 5-fold, 6-fold, 7-fold, 8-fold, 9-fold, 10-fold or more as compared the corresponding molecule. Methods for measuring receptor internalization, endosomal localization and endosomal signaling are known in the art and described herein (see, e.g., Example 2).

### Exemplary TREM2 Activities

As described above, a monovalent anti-TREM2 molecule that binds TfR may have enhanced capabilities for modulating one or more TREM2 activities. Various non-limiting examples of TREM2 activities are described below. For example, in some embodiments, a molecule of the disclosure may modulate levels of sTREM2 protein (e.g., levels of sTREM2 that are shed from the cell surface into an extracellular sample), modulate recruitment or phosphorylation of a kinase that interacts with a TREM2/DAP12 signaling complex (e.g., Syk kinase), and/or modulate one or more activities downstream of the signaling complex, such as phagocytosis, cell growth, cell survival, cell differentiation, cytokine secretion, or cell migration.

In some embodiments, a molecule as described herein may modulate (e.g., enhance) one or more TREM2 activities (e.g., those described herein) that are induced by a ligand. In some embodiments, the ligand is a lipid ligand. Examples of TREM2 lipid ligands include, but are not limited to, 1-palmitoyl-2-(5′-oxo-valeroyl)-sn-glycero-3-phosphocholine (POVPC), 2-Arachidonoylglycerol (2-AG), 7-ketocholesterol (7-KC), 24(S)hydroxycholesterol (24OHC), 25(S)hydroxycholesterol (25OHC), 27-hydroxycholesterol (27OHC), Acyl Carnitine (AC), alkylacylglycerophosphocholine (PAF), α-galactosylceramide (KRN7000), Bis(monoacylglycero)phosphate (BMP), Cardiolipin (CL), Ceramide, Ceramide-1-phosphate (C1P), Cholesteryl ester (CE), Cholesterol phosphate (CP), Diacylglycerol 34:1 (DG 34:1), Diacylglycerol 38:4 (DG 38:4), Diacylglycerol pyrophosphate (DGPP), Dihyrdoceramide (DhCer), Dihydrosphingomyelin (DhSM), Ether phosphatidylcholine (PCe), Free cholesterol (FC), Galactosylceramide (GalCer), Galactosylsphingosine (GalSo), Ganglioside GM1, Ganglioside GM3, Glucosylsphingosine (GlcSo), Hank's Balanced Salt Solution (HBSS), Kdo2-Lipid A (KLA), Lactosylceramide (LacCer), lysoalkylacylglycerophosphocholine (LPAF), Lysophosphatidic acid (LPA), Lysophosphatidylcholine (LPC), Lysophosphatidylethanolamine (LPE), Lysophosphatidylglycerol (LPG), Lysophosphatidylinositol (LPI), Lysosphingomyelin (LSM), Lysophosphatidylserine (LPS), N-Acyl-phosphatidylethanolamine (NAPE), N-Acyl-Serine (NSer), Oxidized phosphatidylcholine (oxPC), Palmitic-acid-9-hydroxy-stearic-acid (PAHSA), Phosphatidylethanolamine (PE), Phosphatidylethanol (PEtOH), Phosphatidic acid (PA), Phosphatidylcholine (PC), Phosphatidylglycerol (PG), Phosphatidylinositol (PI), Phosphatidylserine (PS), Sphinganine, Sphinganine-1-phosphate (Sa1P), Sphingomyelin (SM), Sphingosine, Sphingosine-1-phosphate (So1P), and Sulfatide.

Assays for evaluating certain activities described herein are known in art, for example, as described in PCT publication No. WO2021/146256, which is incorporated by reference herein for all purposes.

Modulation of sTREM2 Shedding

In some embodiments, a monovalent anti-TREM2 molecule that binds TfR may alter levels of sTREM2 protein in a sample, e.g., levels of sTREM2 that are shed from the cell surface into an extracellular sample, to a greater degree than a corresponding molecule that does not bind to a transferrin receptor. In some embodiments, a monovalent anti-TREM2 molecule that binds TfR decreases levels of sTREM2 to a greater degree than a corresponding molecule that does not bind to a transferrin receptor.

In some embodiments, a monovalent anti-TREM2 molecule that binds TfR decreases levels of sTREM2 if the amount of sTREM2 in a treated sample is decreased by at least 10%, at least 20%, at least 30%, at least 40%, at least 50%, at least 60%, at least 70%, at least 80%, at least 90% or more as compared to a control value. In some embodiments, a monovalent anti-TREM2 molecule that binds TfR decreases levels of sTREM2 if the amount of sTREM2 in a treated sample is decreased by at least 2-fold, 3-fold, 4-fold, 5-fold, 6-fold, 7-fold, 8-fold, 9-fold, 10-fold or more as compared to a control value. In some embodiments, the control value is the amount of sTREM2 in an untreated sample (e.g., a supernatant from a TREM2-expressing cell that has not been treated with a molecule as described herein, or a sample from a subject that has not been treated with a molecule as described herein) or a sample treated with an appropriate non-TREM2-binding binding molecule.

In some embodiments, sTREM2 shedding is measured using a sample that comprises a fluid, e.g., blood, plasma, serum, urine, or cerebrospinal fluid. In some embodiments, the sample comprises cerebrospinal fluid. In some embodiments, the sample comprises supernatant from cell cultures (e.g., supernatant from a primary cell or cell line that endogenously expresses TREM2, such as human macrophages, or a primary cell or cell line that has been engineered to express TREM2, e.g., as described in the Examples section below).

In some embodiments, the level of sTREM2 in a sample is measured using an immunoassay. Immunoassays are known in the art and include, but are not limited to, enzyme immunoassays (EIA) such as enzyme multiplied immunoassay (EMIA), enzyme-linked immunosorbent assay (ELISA), microparticle enzyme immunoassay (MEIA), immunohistochemistry (IHC), immunocytochemistry, capillary electrophoresis immunoassays (CEIA), radioimmunoassays (RIA), immunofluorescence, chemiluminescence immunoassays (CL), and electrochemiluminescence immunoassays (ECL). In some embodiments, sTREM2 levels are measuring using an ELISA assay. In some embodiments, sTREM2 levels are measured using an ELISA assay.

### Modulation of Kinase Recruitment or Phosphorylation

In some embodiments, a monovalent anti-TREM2 molecule that binds TfR modulates (e.g., induces) phosphorylation of a kinase that interacts with the TREM2/DAP12 signaling complex (such as, but not limited to, Syk, ZAP70, PI3K, Erk, AKT, or GSK3b) to a greater degree than a corresponding molecule that does not bind to a transferrin receptor. In some embodiments, the molecule modulates (e.g., induces) phosphorylation of a kinase that interacts with the TREM2/DAP12 signaling complex without blocking binding of a native TREM2 ligand. In some embodiments, the molecule modulates (e.g., enhances) phosphorylation of a kinase that interacts with the TREM2/DAP12 signaling complex that is induced by a TREM2 ligand (e.g., a lipid ligand).

In some embodiments, a monovalent anti-TREM2 molecule that binds TfR induces or enhances phosphorylation of Syk to a greater degree than a corresponding molecule that does not bind to a transferrin receptor. In certain embodiments, the molecule increases phosphorylation of Syk by at least about 1.25-fold, 1.5-fold, 1.75-fold, 2-fold, 3-fold, 4-fold, 5-fold, 6-fold, 7-fold, 8-fold, 9-fold, 10-fold or more as compared the corresponding molecule.

In some embodiments, a monovalent anti-TREM2 molecule that binds TfR induces or enhances phosphorylation of Syk if the level of Syk phosphorylation in a sample treated with the molecule is increased by at least 10%, at least 20%, at least 30%, at least 40%, at least 50%, at least 60%, at least 70%, at least 80%, at least 90% or more as compared to a control value. In some embodiments, the molecule induces phosphorylation of Syk if the level of Syk phosphorylation in a sample treated with the molecule is increased by at least 2-fold, 3-fold, 4-fold, 5-fold, 6-fold, 7-fold, 8-fold, 9-fold, 10-fold, or more as compared to a control value. In some embodiments, the control value is the level of Syk phosphorylation in an untreated sample (e.g., a sample comprising a TREM2-expressing cell that has not been treated with a molecule as described herein, or a sample from a subject that has not been treated with a molecule as described herein), or a sample that has been treated with a TREM2 ligand but not a molecule as described herein, or a sample treated with an appropriate non-TREM2-binding molecule.

For detecting and/or quantifying phosphorylation (e.g., Syk phosphorylation) in a sample, in some embodiments, an immunoassay is used. In some embodiments, the immunoassay is an enzyme immunoassay (EIA), enzyme multiplied immunoassay (EMIA), enzyme-linked immunosorbent assay (ELISA), microparticle enzyme immunoassay (MEIA), immunohistochemistry (IHC), immunocytochemistry, capillary electrophoresis immunoassay (CEIA), radioimmunoassay (RIA), immunofluorescence, chemiluminescence immunoassay (CL), or electrochemiluminescence immunoassay (ECL). In some embodiments, phosphorylation is detected and/or quantified using an immunoassay that utilizes an amplified luminescent proximity homogenous assay (AlphaLISA®, PerkinElmer Inc.).

In some embodiments, phosphorylation is measured using a sample that comprises one or more cells, e.g., one or more TREM2-expressing cells (e.g., a primary cell or cell line that endogenously expresses TREM2, such as human macrophages or iPSC-derived microglia, or a primary cell or cell line that has been engineered to express TREM2, e.g., as described in the Examples section below). In some embodiments, the sample comprises a fluid, e.g., blood, plasma, serum, urine, or cerebrospinal fluid. In some embodiments, the sample comprises tissue (e.g., lung, brain, kidney, spleen, nervous tissue, or skeletal muscle) or cells from such tissue. In some embodiments, the sample comprises endogenous fluid, tissue, or cells (e.g., from a human or non-human subject).

### Modulation of Phagocytosis

In some embodiments, a monovalent anti-TREM2 molecule that binds TfR modulates (e.g., enhances) phagocytosis of dead cell debris, tissue debris, amyloid beta particles, or foreign material to a greater degree than a corresponding molecule that does not bind to a transferrin receptor. In some embodiments, a monovalent anti-TREM2 antibody modulates (e.g., enhances) phagocytosis without blocking binding of a native TREM2 ligand. In some embodiments, a monovalent anti-TREM2 antibody modulates (e.g., enhances) phagocytosis that is induced by a TREM2 ligand (e.g., a lipid ligand).

In some embodiments a monovalent anti-TREM2 molecule that binds TfR enhances phagocytosis if the level of phagocytosis in a sample treated with the molecule is increased by at least 10%, at least 20%, at least 30%, at least 40%, at least 50%, at least 60%, at least 70%, at least 80%, at least 90% or more as compared to a control value. In some embodiments, a monovalent anti-TREM2 molecule that binds TfR enhances phagocytosis if the level of phagocytosis in a sample treated with the molecule is increased by at least 2-fold, 3-fold, 4-fold, 5-fold, 6-fold, 7-fold, 8-fold, 9-fold, 10-fold, or more as compared to a control value. In some embodiments, the control value is the level of phagocytosis in an untreated sample, a sample that has been treated with a TREM2 ligand but not a molecule as described herein, or a sample treated with an appropriate non-TREM2-binding molecule.

In some embodiments, phagocytosis is measured using a phagocytosis assay with a labeled substrate. Phagocytosis assays are known in the art. In some embodiments, the phagocytosis assay is performed on a sample comprising cells that endogenously express TREM2, such as human macrophages or microglia. In some embodiments, the phagocytosis assay is performed on a sample comprising cells that have been engineered to express TREM2. In some embodiments, phagocytosis is measured using a human macrophage phagocytosis assay.

### Modulation of Cell Differentiation, Function, Migration, and Survival and Cytokine Secretion

In some embodiments, a monovalent anti-TREM2 molecule that binds TfR modulates (e.g., enhances) cell migration, cell survival, cell function, or cell differentiation (e.g., for myeloid cells, macrophages, and microglia, including iPSC-derived microglia and disease-associated microglia) to a greater degree than a corresponding molecule that does not bind to a transferrin receptor. Disease-associated microglia and methods of detecting disease-associated microglia are described in Keren-Shaul et al., Cell, 2017, 169:1276-1290.

In some embodiments, a monovalent anti-TREM2 molecule that binds TfR enhances cell migration of one or more cell types (e.g., myeloid cells, macrophages, or microglia) to a greater degree than a corresponding molecule that does not bind to a transferrin receptor. In some embodiments, an anti-TREM2 antibody enhances cell survival of one or more cell types (e.g., myeloid cells, macrophages, or microglia) to a greater degree than a corresponding molecule that does not bind to a transferrin receptor. In some embodiments, a monovalent anti-TREM2 molecule that binds TfR enhances cell function of one or more cell types (e.g., myeloid cells, macrophages, or microglia) to a greater degree than a corresponding molecule that does not bind to a transferrin receptor. In some embodiments, a monovalent anti-TREM2 molecule that binds TfR enhances cell differentiation of one or more cell types (e.g., myeloid cells, macrophages, or microglia) to a greater degree than a corresponding molecule that does not bind to a transferrin receptor. In some embodiments, a monovalent anti-TREM2 molecule that binds TfR enhances the migration, survival, function, and/or differentiation of myeloid cells to a greater degree than a corresponding molecule that does not bind to a transferrin receptor. In some embodiments, a monovalent anti-TREM2 molecule that binds TfR enhances the migration, survival, function, and/or differentiation of macrophages to a greater degree than a corresponding molecule that does not bind to a transferrin receptor. In some embodiments, a monovalent anti-TREM2 molecule that binds TfR enhances the migration, survival, function, and/or differentiation of microglia to a greater degree than a corresponding molecule that does not bind to a transferrin receptor. In some embodiments, a monovalent anti-TREM2 molecule that binds TfR enhances microglia activation to a greater degree than a corresponding molecule that does not bind to a transferrin receptor. In some embodiments, a monovalent anti-TREM2 molecule that binds TfR enhances the migration, survival, function, and/or differentiation of disease-associated microglia to a greater degree than a corresponding molecule that does not bind to a transferrin receptor. In some embodiments, a monovalent anti-TREM2 molecule that binds TfR enhances cell migration, cell survival, cell function, or cell differentiation without blocking binding of a native TREM2 ligand. In some embodiments, a monovalent anti-TREM2 molecule that binds TfR enhances cell migration, cell survival, cell function, or cell differentiation that is induced by a TREM2 ligand (e.g., a lipid ligand).

In some embodiments a monovalent anti-TREM2 molecule that binds TfR enhances cell migration, cell survival, cell function, or cell differentiation if the level of activity (e.g., migration, survival, function, or differentiation) in a sample treated with the molecule is increased by at least 10%, at least 20%, at least 30%, at least 40%, at least 50%, at least 60%, at least 70%, at least 80%, at least 90% or more as compared to a control value. In some embodiments, a monovalent anti-TREM2 molecule that binds TfR enhances cell migration, cell survival, cell function, or cell differentiation if the level of activity (e.g., migration, survival, function, or differentiation) in a sample treated with the molecule is increased by at least 2-fold, 3-fold, 4-fold, 5-fold, 6-fold, 7-fold, 8-fold, 9-fold, 10-fold, or more as compared to a control value. In some embodiments, the control value is the level of activity (e.g., migration, survival, function, or differentiation) in an untreated sample (e.g., a sample that has not been treated with a molecule as described herein), a sample that has been treated with a TREM2 ligand but not a molecule as described herein, or a sample treated with an appropriate non-TREM2-binding antibody.

In some embodiments, a given cellular activity or response is evaluated using a biomarker. For example, colony stimulating factor 1 receptor (CSF1R) expression may be used to evaluate microglial activity/response (e.g., myeloid cell survival, proliferation or differentiation). Thus, in certain embodiments, a monovalent anti-TREM2 molecule that binds TfR modulates (e.g., enhances) CSF1R expression to a greater degree than a corresponding molecule that does not bind to a transferrin receptor.

In some embodiments a monovalent anti-TREM2 molecule that binds TfR enhances a microglial response if the level of CSF1R expression in a sample treated with the molecule is increased by at least 10%, at least 20%, at least 30%, at least 40%, at least 50%, at least 60%, at least 70%, at least 80%, at least 90% or more as compared to a control value. In some embodiments, a monovalent anti-TREM2 molecule that binds TfR enhances a microglial response if the level of CSF1R expression in a sample treated with the molecule is increased by at least 2-fold, 3-fold, 4-fold, 5-fold, 6-fold, 7-fold, 8-fold, 9-fold, 10-fold, or more as compared to a control value. In some embodiments, the control value is the level of CSF1R expression in an untreated sample, a sample that has been treated with a TREM2 ligand but not a molecule as described herein, or a sample treated with an appropriate non-TREM2-binding molecule.

In some embodiments, a monovalent anti-TREM2 molecule that binds TfR modulates (e.g., enhances) cytokine secretion and/or expression to a greater degree than a corresponding molecule that does not bind to a transferrin receptor. In certain embodiments, the cytokine is interferon gamma-induced protein 10 (IP-10). In certain embodiments, the cytokine is monocyte chemotactic protein 5 (MCP-5).

In some embodiments a monovalent anti-TREM2 molecule that binds TfR enhances cytokine secretion and/or expression if the level of cytokine secretion/expression in a sample treated with the molecule is increased by at least 10%, at least 20%, at least 30%, at least 40%, at least 50%, at least 60%, at least 70%, at least 80%, at least 90% or more as compared to a control value. In some embodiments, a monovalent anti-TREM2 molecule that binds TfR enhances cytokine secretion and/or expression if the level of cytokine secretion/expression in a sample treated with the molecule is increased by at least 2-fold, 3-fold, 4-fold, 5-fold, 6-fold, 7-fold, 8-fold, 9-fold, 10-fold, or more as compared to a control value. In some embodiments, the control value is the level of cytokine secretion in an untreated sample, a sample that has been treated with a TREM2 ligand but not a molecule as described herein, or a sample treated with an appropriate non-TREM2-binding molecule.

In some embodiments, a biomarker or cytokine secretion/expression is measured using an assay as described herein, such as in Example 4.

In some embodiments, cell migration is measured using a chemotaxis assay. Chemotaxis assays are known in the art. In some embodiments, the cell migration assay (e.g., chemotaxis assay) is performed on a sample comprising cells that endogenously express TREM2, such as human macrophages. In some embodiments, the cell migration assay (e.g., chemotaxis assay) is performed on a sample comprising cells that have been engineered to express TREM2. In some embodiments, cell migration is measured using a human macrophage chemotaxis assay.

In some embodiments, cell survival is measured using a cell viability assay. Cell viability assays are known in the art. In some embodiments, the cell survival assay (e.g., cell viability assay) is performed on a sample comprising cells that endogenously express TREM2, such as human macrophages. In some embodiments, the cell survival assay (e.g., cell viability assay) is performed on a sample comprising cells that have been engineered to express TREM2. In some embodiments, cell survival is measured using a human macrophage viability assay.

In some embodiments, cell function is measured using a functional assay that is appropriate for that cell. For example, in some embodiments, macrophage cell function is evaluated using a phagocytosis assay.

In some embodiments, cell differentiation is measured by evaluating the ability of cells that endogenously express TREM2 to differentiate. For example, in some embodiments, cell differentiation is measured by evaluating the ability of macrophages to differentiate from monocytes.

In some embodiments, activation of microglia is measured in vivo. In some embodiments, microglia activation is measured using TSPO-PET imaging. TSPO-PET imaging methods are known in the art.

In some embodiments, a monovalent anti-TREM2 molecule that binds TfR enhances microglia function without increasing neuroinflammation. Levels of neuroinflammation can be determined by measuring levels of cytokines (e.g., inflammatory cytokines), such as but not limited to TNF-α, IL-1β, IL-6, IL-1ra, TGFβ, IL-15, or IFN-γ. In some embodiments, cytokine levels are measured using immunoassays, for example, an enzyme immunoassay (EIA), enzyme multiplied immunoassay (EMIA), enzyme-linked immunosorbent assay (ELISA), microparticle enzyme immunoassay (MEIA), immunohistochemistry (IHC), immunocytochemistry, capillary electrophoresis immunoassay (CEIA), radioimmunoassay (RIA), immunofluorescence, chemiluminescence immunoassay (CL), or electrochemiluminescence immunoassay (ECL).

### VI. Nucleic Acids, Vectors, and Host Cells

In some embodiments, the monovalent binding molecules as disclosed herein are prepared using recombinant methods. Accordingly, in some aspects, the disclosure provides isolated nucleic acids comprising a nucleic acid sequence encoding any of the monovalent molecules as described herein, or a polypeptide chain(s) comprised within such a molecule, (e.g., any one or more of the CDRs, Fc polypeptides, heavy chain variable regions, and light chain variable regions described herein); vectors comprising such nucleic acids; and host cells into which the nucleic acids are introduced that are used to replicate the molecule-encoding nucleic acids and/or to express the molecules.

In some embodiments, a polynucleotide (e.g., an isolated polynucleotide) comprises a nucleotide sequence encoding a molecule as described herein or encoding a polypeptide chain comprised within a molecule described herein. In some embodiments, the polynucleotide comprises a nucleotide sequence encoding one or more amino acid sequences (e.g., CDR, Fc polypeptide, heavy chain, or light chain sequences) disclosed in Table 8 below.

In some embodiments, the polynucleotide comprises a nucleotide sequence encoding an amino acid sequence having at least 85% sequence identity (e.g., at least 85%, at least 90%, at least 91%, at least 92%, at least 93%, at least 94%, at least 95%, at least 96%, at least 97%, at least 98%, or at least 99% sequence identity) to a sequence (e.g., a CDR, Fc polypeptide, heavy chain, or light chain sequence) disclosed in Table 8 below. For example, certain embodiments provide a polynucleotide comprising a nucleotide sequence encoding a light chain, an Fc polypeptide and/or heavy chain described herein. Certain embodiments also provide a plurality of polynucleotides, wherein the plurality comprises a polynucleotide comprising a nucleotide sequence encoding a light chain described herein, a polynucleotide comprising a nucleotide sequence encoding an Fc polypeptide as described herein, and a polynucleotide comprising a nucleotide sequence encoding a heavy chain described herein (e.g., a heavy chain comprising a Fc polypeptide capable of binding to a TfR; or a heavy chain linked to a second binding region).

For example, certain embodiments provide an isolated polynucleotide comprising one or more nucleotide sequences encoding any one of SEQ ID NOS: 48, 51, 94, 96, 85, 86, 88, 141, 171, and 54.

Thus, certain embodiments provide an isolated polynucleotide comprising a nucleotide sequence encoding SEQ ID NOS:94, 88, and 54. Certain embodiments also provide a plurality of polynucleotides, wherein the plurality comprises a polynucleotide comprising a nucleotide sequence encoding SEQ ID NO:54, a polynucleotide comprising a nucleotide sequence encoding SEQ ID NO:88, and a polynucleotide comprising a nucleotide sequence encoding a SEQ ID NO:94.

Certain embodiments provide an isolated polynucleotide comprising a nucleotide sequence encoding SEQ ID NOS:96, 88, and 54. Certain embodiments also provide a plurality of polynucleotides, wherein the plurality comprises a polynucleotide comprising a nucleotide sequence encoding SEQ ID NO:54, a polynucleotide comprising a nucleotide sequence encoding SEQ ID NO:88, and a polynucleotide comprising a nucleotide sequence encoding a SEQ ID NO:96.

Certain embodiments provide an isolated polynucleotide comprising a nucleotide sequence encoding SEQ ID NOS:48, 85, and 54. Certain embodiments also provide a plurality of polynucleotides, wherein the plurality comprises a polynucleotide comprising a nucleotide sequence encoding SEQ ID NO:54, a polynucleotide comprising a nucleotide sequence encoding SEQ ID NO:85, and a polynucleotide comprising a nucleotide sequence encoding a SEQ ID NO48.

Certain embodiments provide an isolated polynucleotide comprising a nucleotide sequence encoding SEQ ID NOS:48, 86, and 54. Certain embodiments also provide a plurality of polynucleotides, wherein the plurality comprises a polynucleotide comprising a nucleotide sequence encoding SEQ ID NO:54, a polynucleotide comprising a nucleotide sequence encoding SEQ ID NO:86, and a polynucleotide comprising a nucleotide sequence encoding a SEQ ID NO:48.

Certain embodiments provide an isolated polynucleotide comprising a nucleotide sequence encoding SEQ ID NOS:54, 141, and 171. Certain embodiments also provide a plurality of polynucleotides, wherein the plurality comprises a polynucleotide comprising a nucleotide sequence encoding SEQ ID NO:54, a polynucleotide comprising a nucleotide sequence encoding SEQ ID NO:141, and a polynucleotide comprising a nucleotide sequence encoding a SEQ ID NO:171.

Certain embodiments provide an isolated polynucleotide comprising a nucleotide sequence encoding SEQ ID NOS:51, 86, and 54. Certain embodiments also provide a plurality of polynucleotides, wherein the plurality comprises a polynucleotide comprising a nucleotide sequence encoding SEQ ID NO:54, a polynucleotide comprising a nucleotide sequence encoding SEQ ID NO:86, and a polynucleotide comprising a nucleotide sequence encoding a SEQ ID NO:51.

In some embodiments, a polynucleotide as described herein is operably linked to a heterologous nucleic acid, e.g., a heterologous promoter.

Suitable vectors containing polynucleotide(s) encoding molecules of the present disclosure, or fragments thereof, include cloning vectors and expression vectors are provided. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicate in the host cells either as episomes or as an integral part of the chromosomal DNA. Suitable expression vectors include but are not limited to plasmids, viral vectors, including adenoviruses, adeno-associated viruses, retroviruses, and any other vector.

Suitable host cells for cloning or expressing a polynucleotide or vector as described herein include prokaryotic or eukaryotic cells. In some embodiments, the host cell is prokaryotic. In some embodiments, the host cell is eukaryotic, e.g., Chinese Hamster Ovary (CHO) cells or lymphoid cells. In some embodiments, the host cell is a human cell, e.g., a Human Embryonic Kidney (HEK) cell.

In another aspect, methods of making a monovalent anti-TREM2 molecule that binds a TfR are provided. In some embodiments, the method includes culturing a host cell as described herein (e.g., a host cell expressing a polynucleotide or vector as described herein) under conditions suitable for expression of the molecule. In some embodiments, the molecule is subsequently recovered from the host cell (or host cell culture medium).

### VII. Therapeutic Methods Using Monovalent Binding Molecules

In another aspect, therapeutic methods using a monovalent anti-TREM2 molecule that binds a TfR as disclosed herein are provided. In some embodiments, methods of treating a disease or modulating one or more activities TREM2 are provided. In some embodiments, the method comprises administering to a subject a monovalent anti-TREM2 molecule that binds a TfR as described herein, or a pharmaceutical composition comprising such a molecule.

In some embodiments, methods of treating a neurodegenerative disease are provided. In some embodiments, methods of modulating one or more TREM2 activities (e.g., in a subject having a neurodegenerative disease) are provided.

In some embodiments, methods of treating a neurodegenerative disease are provided. In some embodiments, the neurodegenerative disease is selected from the group consisting of Alzheimer's disease, primary age-related tauopathy, progressive supranuclear palsy (PSP), frontotemporal dementia, frontotemporal dementia with parkinsonism linked to chromosome 17, argyrophilic grain dementia, amyotrophic lateral sclerosis, amyotrophic lateral sclerosis/parkinsonism-dementia complex of Guam (ALS-PDC), corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, hereditary diffuse leukoencephalopathy with spheroids (HDLS), Huntington's disease, inclusion-body myositis, multiple system atrophy, myotonic dystrophy, Nasu-Hakola disease, neurofibrillary tangle-predominant dementia, Niemann-Pick disease type C, pallido-ponto-nigral degeneration, Parkinson's disease, Pick's disease, postencephalitic parkinsonism, prion protein cerebral amyloid angiopathy, progressive subcortical gliosis, subacute sclerosing panencephalitis, adult-onset leukoencephalopathy with axonal spheroids and pigmented glia (ALSP), and tangle only dementia. In some embodiments, the neurodegenerative disease is Alzheimer's disease. In some embodiments, the neurodegenerative disease is Nasu-Hakola disease. In some embodiments, the neurodegenerative disease is frontotemporal dementia. In some embodiments, the neurodegenerative disease is Parkinson's disease. In some embodiments, the neurodegenerative disease is ALSP. In some embodiments, the method comprises administering to a subject an isolated monovalent antibody, or an antigen-binding fragment thereof, that specifically binds to a human TREM2 protein, e.g., a monovalent anti-TREM2 antibody as described herein, or a pharmaceutical composition comprising a monovalent anti-TREM2 antibody as described herein.

In some embodiments, a monovalent anti-TREM2 molecule that binds a TfR as described herein (or pharmaceutical composition thereof) as described herein is used in treating a neurodegenerative disease that is characterized by a mutation in TREM2. In some embodiments, the neurodegenerative disease that is characterized by a mutation in TREM2 is Alzheimer's disease, e.g., Alzheimer's disease that is characterized by a R47H mutation in TREM2.

In some embodiments, methods of modulating one or more TREM2 activities in a subject (e.g., a subject having a neurodegenerative disease) are provided. In some embodiments, the method comprises modulating levels of sTREM2; modulating recruitment or phosphorylation of a kinase that interacts with a TREM2/DAP12 signaling complex (e.g., Syk kinase); modulating phagocytosis (e.g., phagocytosis of cell debris, amyloid beta particles, etc.); modulating cell migration (e.g., migration of myeloid cells, macrophages, microglia, and disease associated microglia); and/or modulating cell differentiation (e.g., for myeloid cells, macrophages, microglia, and disease associated microglia). In some embodiments, methods of enhancing one or more TREM2 activities in a subject having a neurodegenerative disease are provided. In some embodiments, methods of decreasing levels of sTREM2 in a subject having a neurodegenerative disease are provided. In some embodiments, the method of modulating one or more TREM2 activities in a subject comprises administering to the subject a monovalent anti-TREM2 molecule that binds a TfR as described herein, or a pharmaceutical composition comprising such a molecule.

In some embodiments, the subject to be treated is a human, e.g., a human adult or a human child.

### VIII. Pharmaceutical Compositions

In another aspect, pharmaceutical compositions comprising a monovalent anti-TREM2 molecule that binds a TfR as described herein are provided. In some embodiments, the pharmaceutical compositions are for use in treating a disease, such as a neurodegenerative disease.

In some embodiments, a pharmaceutical composition comprises a monovalent anti-TREM2 molecule that binds a TfR as described herein and further comprises one or more pharmaceutically acceptable carriers and/or excipients. A pharmaceutically acceptable carrier includes any solvents, dispersion media, or coatings that are physiologically compatible and that does not interfere with or otherwise inhibit the activity of the active agent. Various pharmaceutically acceptable excipients are well-known in the art.

A monovalent anti-TREM2 molecule that binds a TfR as described herein can be formulated for parenteral administration by injection, e.g., by bolus injection or continuous infusion.

### IX. Certain Alternative Embodiments

Certain alternative embodiments of the disclosure include certain bivalent anti-TREM2 molecules, which comprise a second binding region that specifically binds to a transferrin receptor (TfR), and that may be based on sequences described herein. For example, in some embodiments, a bivalent anti-TREM2 binding molecule may comprise one or more complementarity determining region (CDR) sequences, a heavy chain variable region sequence, and/or a light chain variable region sequence as disclosed herein. In some embodiments, a heavy chain sequence, or a portion thereof, and/or a light chain sequence, or a portion thereof, is derived from an anti-TREM2 antibody described herein (e.g., Clone CL0020306, Clone CL0020188, Clone CL0020307, Clone A, Clone B, Clone C or Clone D). The CDR, heavy chain variable region, and light chain variable region amino acid sequences of these clones are set forth in Table 8. The bivalent anti-TREM2 molecules may have a structure/sequence based on a corresponding monovalent molecule as described herein but would have two sets of variable regions that have affinity for TREM2. Similarly, TfR binding in such a bivalent molecule may be based on a second binding region that has modifications and/or sequences described herein (see, e.g., Table 8). For Example, certain embodiments provide a bivalent anti-TREM2 molecule that comprises a second binding region as described herein linked to the C-terminus of one of its heavy chains. For example, the second binding region can be an scFab or an scFv.

In certain embodiments, the molecule comprises:
(i) a first heavy chain (HC) linked to a second binding region (SBR) comprising 1) a V_{H} comprising a sequence that has at least 85%, 90%, 95% or 100% identity to any one of SEQ ID NOS:24, 113, 121, 129, and 136; 2) the first Fc polypeptide comprising a sequence having at least 85%, 90%, 95% or 100% identity to SEQ ID NO:166; and 3) the SBR comprising an scFv, an scFab, or a SBR V_{H} comprising a sequence that has at least 85%, 90%, 95% or 100% identity to SEQ ID NO:156 and a SBR V_{L} comprising a sequence that has at least 85%, 90%, 95% or 100% identity to SEQ ID NO:151;
(ii) a second HC comprising 1) a V_{H} comprising a sequence that has at least 85%, 90%, 95% or 100% identity to any one of SEQ ID NOS:24, 113, 121, 129, and 136; and 2) a second Fc polypeptide comprising a sequence having at least 85%, 90%, 95% or 100% identity to SEQ ID NO:140 or 163; and
(iii) two light chains each independently comprising a V_{L} comprising a sequence that has at least 85%, 90%, 95% or 100% identity to any one of SEQ ID NOS:22, 109, 117, 125, and 132.

In certain embodiments, the molecule comprises:
(i) a first heavy chain (HC) linked to a second binding region (SBR) comprising 1) a V_{H} comprising a sequence selected from the group consisting of SEQ ID NOS:24, 113, 121, 129, and 136; 2) the first Fc polypeptide comprising a sequence having at least 85%, 90%, 95% or 100% identity to SEQ ID NO:166 (e.g., 100% identity to SEQ ID NO:166); and 3) the SBR comprising an scFv, an scFab, or a SBR V_{H} sequence comprising SEQ ID NO:156 and a SBR V_{L} sequence comprising SEQ ID NO:151;
(ii) a second HC comprising 1) a V_{H} comprising a sequence selected from the group consisting of SEQ ID NOS:24, 113, 121, 129, and 136; and 2) a second Fc polypeptide comprising a sequence having at least 85%, 90%, 95% or 100% identity to SEQ ID NO:140 (e.g., 100% identity to SEQ ID NO:140); and
(iii) two light chains each comprising a V_{L} comprising a sequence selected from the group consisting of SEQ ID NOS:22, 109, 117, 125, and 132.

In certain embodiments, the molecule comprises:
(i) a first heavy chain (HC) linked to the second binding region (SBR) comprising 1) a V_{H} comprising a sequence selected from the group consisting of SEQ ID NOS:24, 113, 121, 129, and 136; 2) the first Fc polypeptide comprising a sequence having at least 85%, 90%, 95% or 100% identity to SEQ ID NO:166 (e.g., 100% identity to SEQ ID NO:166); and 3) the SBR comprising an scFv, scFab, or a SBR V_{H} sequence comprising SEQ ID NO:156 and a SBR V_{L} sequence comprising SEQ ID NO:151;
(ii) a second HC comprising 1) a V_{H} comprising a sequence selected from the group consisting of SEQ ID NOS:24, 113, 121, 129, and 136; and 2) a second Fc polypeptide comprising a sequence having at least 85%, 90%, 95% or 100% identity to SEQ ID NO:163 (e.g., 100% identity to SEQ ID NO:163); and
(iii) two light chains each comprising a V_{L} comprising a sequence selected from the group consisting of SEQ ID NOS:22, 109, 117, 125, and 132.

In certain embodiments:
(a) each V_{H} comprises SEQ ID NO:24 and each V_{L} comprises SEQ ID NO:22;
(b) each V_{H} comprises SEQ ID NO:113 and each V_{L} comprises SEQ ID NO: 109;
(c) each V_{H} comprises SEQ ID NO:121 and each V_{L} comprises SEQ ID NO: 117;
(d) each V_{H} comprises SEQ ID NO:129 and each V_{L} comprises SEQ ID NO: 125; or
(e) each V_{H} comprises SEQ ID NO:136 and each V_{L} comprises SEQ ID NO:132.

In certain embodiments, the molecule comprises:
(i) a first heavy chain (HC) linked to the second binding region (SBR), which comprises from N-terminus to C-terminus: 1) the heavy chain comprising a sequence having at least 90%, 95% or 100% identity to SEQ ID NO:168; 2) a first polypeptide linker; and 3) the SBR, which comprises from N-terminus to C-terminus: a) a SBR light chain comprising a sequence having at least 90%, 95% or 100% identity to SEQ ID NO: 150; b) a second polypeptide linker; and c) a Fd region comprising a sequence having at least 90%, 95% or 100% identity to SEQ ID NO: 155;
(ii) a second HC comprising a sequence having at least 90%, 95% or 100% identity to SEQ ID NO: 142 or 165; and
(iii) a first and a second light chain that each independently comprise a sequence having at least 90%, 95% or 100% identity to SEQ ID NO:54. In certain embodiments, the first and/or the second polypeptide linker are a linker described herein, such as a Gly- and/or Ser-rich linker.

In certain embodiments, the molecule comprises:
(i) a first heavy chain (HC) linked to the second binding region (SBR), which comprises from N-terminus to C-terminus: 1) the first heavy chain comprising or consisting of the amino acid sequence set forth in SEQ ID NO: 168; 2) a first polypeptide linker; and 3) the SBR, which comprises from N-terminus to C-terminus: a) a SBR light chain comprising or consisting of the amino acid sequence set forth in SEQ ID NO: 150; b) a second polypeptide linker; and c) a Fd region comprising or consisting of the amino acid sequence set forth in SEQ ID NO: 155;
(ii) a second HC that comprises or consists of the amino acid sequence set forth in SEQ ID NO: 142; and
(iii) a first and a second light chain (LC) that each comprises or consists of the amino acid sequence set forth in SEQ ID NO:54. In certain embodiments, the first and/or the second polypeptide linker are a linker described herein, such as a Gly- and/or Ser-rich linker.

In certain embodiments, the molecule comprises:
(i) a first heavy chain (HC) linked to the second binding region (SBR), which comprises from N-terminus to C-terminus: 1) the first heavy chain comprising or consisting of the amino acid sequence set forth in SEQ ID NO:168; 2) a first polypeptide linker; and 3) the SBR, which comprises from N-terminus to C-terminus: a) a SBR light chain comprising or consisting of the amino acid sequence set forth in SEQ ID NO: 150; b) a second polypeptide linker; and c) a Fd region comprising or consisting of the amino acid sequence set forth in SEQ ID NO: 155;
(ii) a second HC that comprises or consists of the amino acid sequence set forth in SEQ ID NO: 165; and
(iii) a first and a second light chain (LC) that each comprise or consist of the amino acid sequence set forth in SEQ ID NO:54. In certain embodiments, the first and/or the second polypeptide linker are a linker described herein, such as a Gly- and/or Ser-rich linker.

In certain embodiments, the molecule comprises:
(i) a first heavy chain (HC) linked to the second binding region that comprises a sequence having at least 90%, 95% or 100% identity to SEQ ID NO:171;
(ii) a second HC that comprises a sequence having at least 90%, 95% or 100% identity to SEQ ID NO: 142 or 165; and
(iii) a first and a second light chain (LC) that each independently comprise a sequence having at least 90%, 95% or 100% identity to SEQ ID NO:54.

In certain embodiments, the molecule comprises:
(i) a first heavy chain (HC) linked to the second binding region that comprises or consists of SEQ ID NO:171;
(ii) a second HC that comprises or consists of the amino acid sequence set forth in SEQ ID NO: 142; and
(iii) a first and a second light chain (LC) that each comprise or consist of the amino acid sequence set forth in SEQ ID NO:54.

In certain embodiments, the molecule comprises:
(i) a heavy chain (HC) linked to the second binding region that comprises or consists of SEQ ID NO:171;
(ii) a second HC that comprises or consists of the amino acid sequence set forth in SEQ ID NO: 165; and
(iii) a first and a second light chain (LC) that each comprise or consist of the amino acid sequence set forth in SEQ ID NO:54.

Certain embodiments provide isolated nucleic acids comprising a nucleic acid sequence encoding such bivalent molecules as described herein, or a polypeptide chain(s) comprised within such a molecule, (e.g., any one or more of the CDRs, Fc polypeptides, heavy chain variable regions, and light chain variable regions described herein); vectors comprising such nucleic acids; and host cells into which the nucleic acids are introduced that are used to replicate the molecule-encoding nucleic acids and/or to express the molecules.

Bivalent molecules described herein may also be incorporated into a pharmaceutical composition as described herein and may also be used in a method as described herein.

### X. Examples

The present disclosure will be described in greater detail by way of specific examples. The following examples are offered for illustrative purposes only, and are not intended to limit the disclosure in any manner.

### Example 1. Generation of Anti-TREM2 Antibodies Having Modified Fc Polypeptides

### Generation of Recombinant Antibodies

The standard bivalent IgG anti-TREM2 (BV) antibody containing the effector knock-out substitutions was generated by cloning the variable domains of the TREM2 antibody into the human IgG1 L234A/L235A (LALA), and human kappa chain expression vectors. The standard monovalent IgG anti-TREM2 (MV) antibody was similarly generated but contained L234A/L235A/P329G, N297G and M428L; N434S mutations. Additionally, the Fc linked to the Fab region comprised a “knob” (T366W) mutation; and the sequence encoding the other Fc polypeptide comprised “hole” (T366S/L368A/Y407V) mutations. To generate ATV:TREM2 in either BV or monovalent MV Fab format, an Fc domain was replaced by the engineered Fc sequence (Kariolis et al., Sci Transl Med. (2020) 12(545):eaay1359) with the effector knock-out substitutions. Additionally, the sequence encoding the Fc polypeptide engineered to bind TfR also contained a “knob” (T366W) mutation; and the sequence encoding the other Fc polypeptide that lacked TfR binding mutations comprised “hole” (T366S/L368A/Y407V) mutations. These coding sequences also contained “LS” (M428L; N434S) mutations, and the MV format further contained N297G and P329G mutations. The light chain, heavy chain and Fc sequences for the various formats are set forth in Table 1.

TABLE 1
Anti-TREM2 and ATV:TREM2 Sequences

| | | Second Heavy Chain or | |
|---|---|---|---|
| Antibody | Heavy Chain | Fc Polypeptide | Light Chain |
| anti-TREM2 (BV) | SEQ ID NO: 83 | SEQ ID NO: 83 | SEQ ID NO: 54 |
| anti-TREM2 MV | SEQ ID NO: 174 or 175 | SEQ ID NO: 88 or 100 | SEQ ID NO: 54 |
| ATV:TREM2 (BV) | SEQ ID NO: 48 or 69 | SEQ ID NO: 53 or 73 | SEQ ID NO: 54 |
| ATV:TREM2 MV | SEQ ID NO: 94 or 106 | SEQ ID NO: 88 or 100 | SEQ ID NO: 54 |

The first 10 amino acids of SEQ ID NOS:88 and 100 are a portion of an IgG1 hinge region. The CH2 domain sequence starts at position 11 of SEQ ID NOs:88 and 100, respectively.

The heavy chain and/or Fc polypeptide of the antibodies, including anti-TREM2 (BV), may be further processed during cell culture production, such that the C-terminal Lys residue is removed (i.e., the Lys residue at position 447, according to the EU numbering scheme). For example, as used herein, the term ATV:TREM2 MV may be used may be used to refer to monovalent antibodies having unprocessed sequences (i.e., SEQ ID NOs:94 and 88); monovalent antibodies comprising one or more processed sequences (i.e., selected from SEQ ID NOs: 106 and 100); or to a composition comprising a mixture of processed and unprocessed monovalent antibodies.

### Expression and Purification of Antibodies

Anti-TREM2 antibody, ATV:TREM2 and isotype controls were expressed via transient transfection of Expi293 cell line (Thermo Fisher Scientific) adapted to BalanCD HEK293 media (Irvine Scientific) according to manufacturer's instructions. Cultures were co-transfected with plasmids encoding for standard monoclonal antibody (2-chain): 1:1 Heavy Chain (HC):Light Chain (LC); and antibody transport vehicle (ATV) molecule: 1:1:2 Knob:Hole:Light Chain (LC).

Anti-TREM2 antibody, ATV:TREM2 and isotype controls were purified to homogeneity from serum-free BCD293 cultures by a series of chromatographic steps. Supernatants were loaded onto a 1×PBS equilibrated HiTrap MabSelect PrismA affinity column (Cytiva using an Akta Pure System), the column was then washed with 5 column volumes (CVs) of 1×PBS and 0.1% Triton X-100, followed by 10 CV of 1×PBS wash. Bound proteins were eluted using 0.1 M sodium citrate pH3.6 and 150 mM sodium chloride. Immediately after elution, Protein A eluate was neutralized to pH6.5 with 1M Tris pH8. Neutralized Protein A eluate was conditioned with 50 mM sodium acetate pH6.5 prior cation-exchange chromatography. A linear gradient with, 0.5M sodium chloride and 50 mM sodium acetate pH6.5 was used to elute proteins from SP HP resin (Cytiva). Final fractions with a high degree of purity, as assessed by analytical size-exclusion chromatography (SEC) and/or microcapillary electrophoresis (Caliper), were pooled, concentrated and dialyzed into formulation buffer of 10 mM sodium acetate pH5.5, 6% sucrose and/or 1×PBS for cell-based functional assay. Preparations were stored at 4° C. or −80° C. prior to use and routinely analyzed by SEC, SPR and for endotoxin content.

### Example 2. Characterization of Anti-TREM2 MV Antibody

### Results

TfR-TREM2 Interactions Mediated by ATV:TREM2 Enhance pSyk Signaling and Promotes TREM2 Internalization

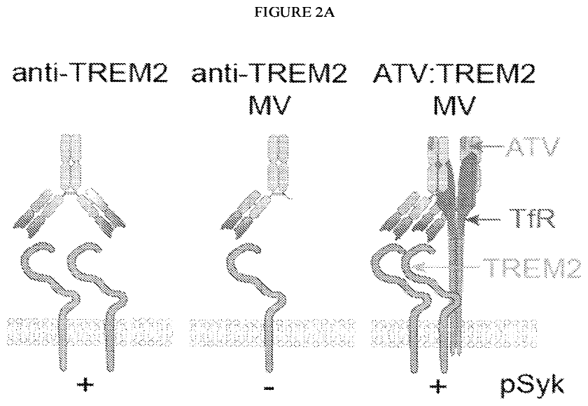

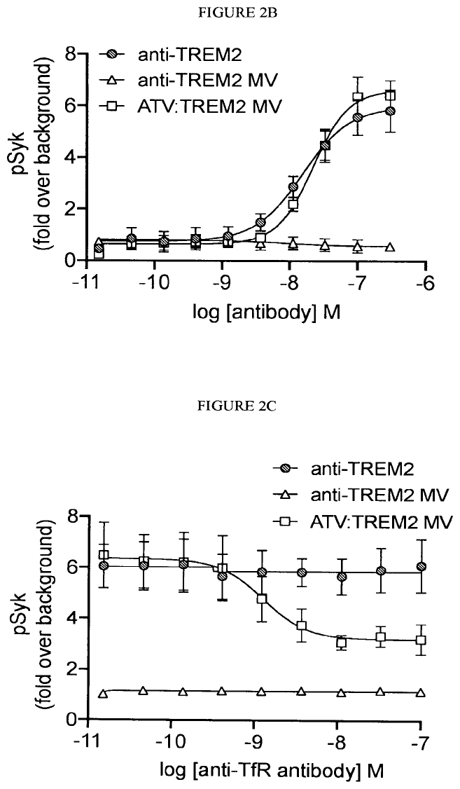

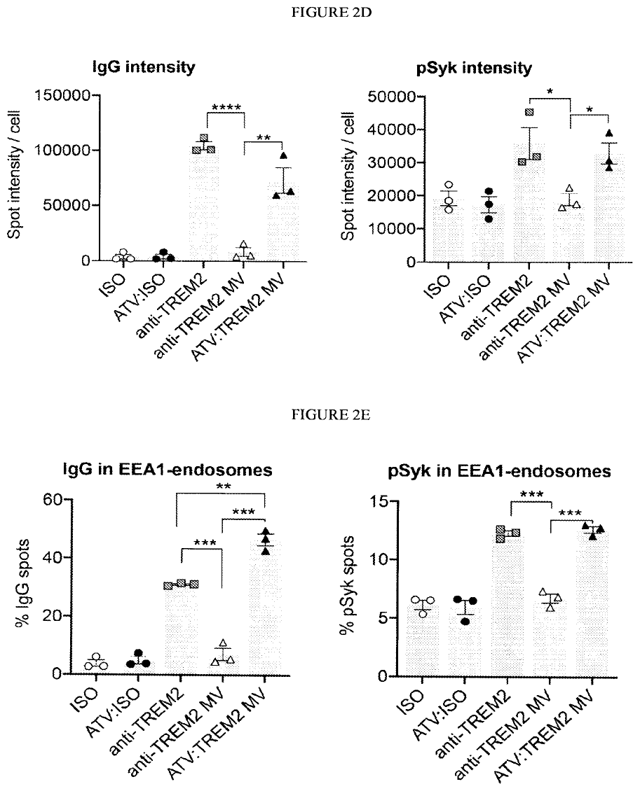

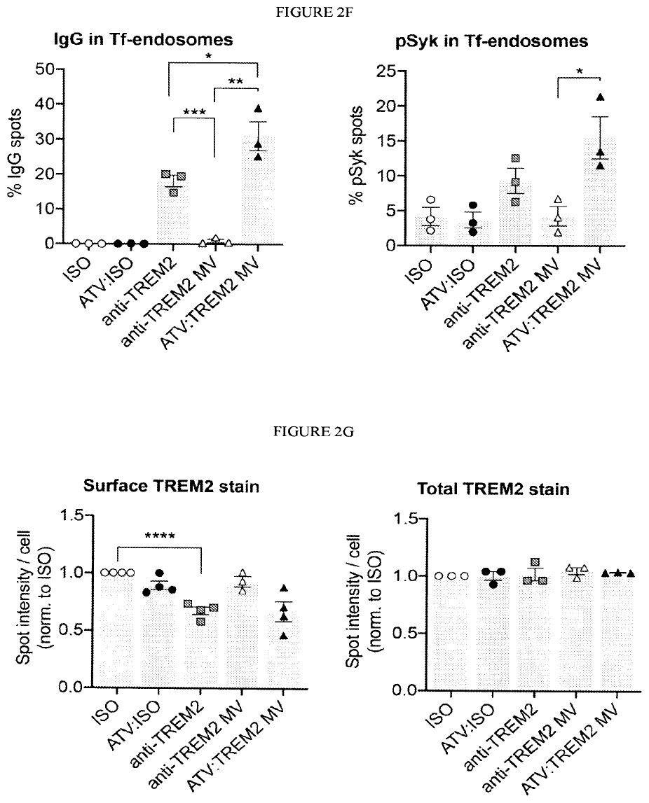

Previous studies demonstrated TREM2 activating antibodies require bivalent binding, whereas monovalent Fab displayed no activity (FIG. 2A, Schlepckow et al., EMBO Mol Med. (2020) 12(4):e11227; Ellwanger et al., Proc Natl Acad Sci USA. (2021) 118(3):e2017742118). The impact of bivalent (BV) and monovalent (MV) versions of anti-TREM2 and ATV:TREM2 on pSyk signaling was evaluated to determine whether MV or BV versions of ATV:TREM2 could display differential activity by binding of both TREM2 and TfR (see, Example 1 for antibody sequences).

Consistent with previous results, anti-TREM2 MV showed no activity (FIG. 2B; Table 2). Surprisingly, ATV:TREM2 MV displayed similar maximal response and potency as anti-TREM2 BV, and ATV:TREM2 BV showed improved maximal signaling and potency (FIG. 2B; Table 2), demonstrating that ATV enhances TREM2 Fab activity.

To confirm that the signaling impacts of TfR-TREM2 interactions were TfR dependent, a high affinity TfR apical domain antibody was used to compete with ATV binding. The anti-TfR antibody inhibited ATV:TREM2 MV and BV pSyk potentiation in a dose-dependent manner with no effect on anti-TREM2 BV or MV (FIG. 2C; Table 2). Thus, ATV-TfR binding is required for enhanced ATV:TREM2 signaling.

TABLE 2

| | | Emax pSyk | | Imax TfR |
|---|---|---|---|---|
| | EC50 pSyk | activation | IC50 TfR | blocking |
| Molecule | activation | (FOB) | blocking | (FOB) |
| Anti-TREM2 | 15.5 nM | 5.97 | No fit | 5.85 |
| Anti-TREM2 MV | No fit | 0.80 | No fit | 1.17 |
| ATV:TREM2 MV | 22.1 nM | 6.62 | 1.3 nM | 3.20 |
| ATV:TREM2 BV | 6.0 nM | 12.35 | 3.1 nM | 16.29 |

### ATV Driven Internalization of TREM2-Antibody Complex Results in Endosomal Signaling

Since TfR undergoes constitutive internalization and endosomal recycling, whether ATV affects intracellular trafficking or signaling localization was investigated. TREM2 was tracked by immunofluorescent (IF) imaging in hTREM2-DAP12 HEK293 cells and rapid downregulation of surface TREM2 was observed within 10 min of ATV:TREM2 BV treatment (FIG. 2G; Table 3). Total cellular TREM2 levels detected by IF upon permeabilization remained constant, suggesting reduced cell surface TREM2 reflects receptor internalization, and not degradation (FIG. 2G; Table 3).

TABLE 3

| | Surface TREM2 | Total TREM2 |
|---|---|---|
| Molecule | (vs ISO) | (vs ISO) |
| ISO | 1.00 | 1.00 |
| ATV:ISO | 0.89 | 1.00 |
| Anti-TREM2 | 0.67 | 1.02 |
| Anti-TREM2 MV | 0.93 | 1.05 |
| ATV:TREM2 MV | 0.67 | 1.04 |
| ATV:TREM2 BV | 0.29 | 1.06 |

Subcellular localization and trafficking of therapeutic TREM2 antibodies by IF was also investigated. Both ATV:TREM2 BV and MV were detected intracellularly, while anti-TREM2 showed surface localization. Quantification demonstrated increased total amount of ATV:TREM2 bound or internalized compared to anti-TREM2 in both BV and MV ATVs (FIG. 2D; Table 4). Consistent with the ELISA results (FIG. 2B), both ATV:TREM2 BV and MV increased pSyk compared to anti-TREM2 when compared respective to valency as detected by IF (FIG. 2D; Table 4). These data indicate that TfR binding causes internalization of the antibody due to endocytosis, as observed by IgG punctae that colocalize with EEA1 (an early endosomal marker), and TfR, a recycling endosomal receptor, which may contribute to enhanced signaling. These data suggest that ATV:TREM2 mediates enhanced activity not solely by increasing antibody bound and/or taken up by the cell, but also by affecting intracellular trafficking of TREM2.

TABLE 4

| | IgG intensity | pSyk intensity |
|---|---|---|
| Antibody | (spot intensity/cell) | (spot intensity/cell) |
| ISO | 3,868 | 19,203 |
| ATV:ISO | 4,167 | 17,314 |
| Anti-TREM2 | 104,476 | 35,829 |
| Anti-TREM2 MV | 5,320 | 18,834 |
| ATV:TREM2 MV | 73,178 | 32,829 |
| ATV:TREM2 BV | 156,606 | 53,317 |

To examine the relationship between TREM2 trafficking and signaling, antibody (IgG) and pSyk co-localization to EEA1 endosomes and TfR⁺ recycling endosomes were quantified using a mask-based imaging analysis (FIG. 2E, 2F, Table 5). ATV:TREM2 MV and pSyk were detected in EEA1⁺ and TfR⁺ endosomes compared to anti-TREM2 MV which was detected at levels similar to negative isotype controls (FIG. 2E, 2F, Table 5). Notably, pSyk was predominantly found in membrane localized puncta. The co-localization of pSyk signaling with EEA1 in a TfR-binding dependent manner suggested that signaling can originate from both endosomes as well as the plasma membrane. These data indicated that ATV:TREM2 can mediate antibody internalization and endosomal localization, which contributes in part to enhanced pSyk signaling. Taken together, these mechanistic studies demonstrate dual mechanisms of action in which ATV enhances TREM2 antibody function via TfR-TREM2 binding, as well as internalization and endosomal signaling, supporting differentiated mechanisms of action for ATV:TREM2 vs. anti-TREM2.

TABLE 5

| | % IgG in | % pSyk in | | |
|---|---|---|---|---|
| | EEA1 | EEA1 | % IgG in Tf- | % pSyk in Tf- |
| Antibody | endosomes | endosomes | endosomes | endosomes |
| ISO | 3.89 | 6.15 | 0.03 | 4.18 |
| ATV:ISO | 4.94 | 5.95 | 0.08 | 3.70 |
| Anti-TREM2 | 31.09 | 12.27 | 18.09 | 9.32 |
| Anti-TREM2 MV | 7.10 | 6.73 | 0.91 | 4.26 |
| ATV:TREM2 MV | 46.52 | 12.61 | 31.05 | 15.48 |
| ATV:TREM2 BV | 59.85 | 25.74 | 51.26 | 29.90 |

In summary, it was determined using cell-based signaling assay that ATV enhanced both BV and MV TREM2 Fab activity through two distinct, yet non-exclusive mechanisms: 1) TREM2 receptor signaling, in which ATV binding to TfR increases TREM2 MV Fab activation of TREM2; and 2) increased trafficking, in which TfR binding facilitates TREM2 internalization and promotes endosomal signaling.

### Materials and Methods

### Cell Culture and Generation of TREM2-DAP12 Overexpression HEK293 Stable Cell Lines

A HEK293 cell line stably expressing human or cynomolgus monkey TREM2/DAP12 was generated by transfecting the cells with the dual promoter pBudCE4.1vector driving expression of wild type TREM2 under the CMV promoter and DAP12 the under the EF1a promoter. Stable clones were isolated after zeocin selection (800 μg/mL for 10 days), and cell surface TREM2 was detected by flow cytometry (APC-conjugated rat anti-human/mouse-TREM2 monoclonal antibody, R&D, catalog no MAB17291). The clone showing the highest wild type TREM2 expression level was selected.

pSyk Signaling

### Dosing of Antibodies in Cells

The day before assay, hTREM2-DAP12 HEK293 cells were plated at 50,000 cells/well on a 96-well plate coated with poly-D-lysine. Antibodies were serially diluted into PBS starting at 300 nM and proceeding in a 10-point titration with 3-fold dilutions between points. For TfR blocking assay, TREM2 antibody was mixed with a custom-made monoclonal TfR antibody that binds the ATV epitope (KD ˜0.9 nM). The TfR antibody solution was serially diluted starting at 1 uM, following 3-fold dilution for 9 points. The antibody solution was added with TREM2 antibody at 100 nM and was mixed briefly before being incubated at 37° C. for 30 min. Before treatment with antibodies, the cells were washed 3 times with HBSS using a Biotek EL406 plate washer, after which 40 μL per well of the antibody/PBS solution was added using a Bravo liquid handler. The cell plate was then transferred to a 37° C. incubator for 5 minutes. The antibody solution was removed by flicking the plate, and 35 μL lysis buffer (Cell Signaling Technologies, CST) containing 1 μM PMSF was added using the liquid handler. The lysate was then frozen at −80° C.

pSyk AlphaLISA

Lysates were assayed for pSyk using the standard protocol for the Perkin Elmer pSyk Alpha Lisa kit. 10 μL of lysate/well was transferred to a white opaque 384 well Optiplate (Perkin Elmer). Next 5 μL of Acceptor Mix (containing the working solution of acceptor beads) was added per well followed by sealing of plates with foil seals and incubation 1 hour at room temperature. After this 5 μL of Donor Mix (containing the working solution of donor beads) was added to each well. Plates were again sealed and incubated 1 hour at room temperature, then plates were read using Alpha Lisa settings on a Perkin Elmer EnVision plate reader.

### TREM2 Receptor and ATV:TREM2 Trafficking Endosomal Signaling by Immunofluorescence Microscopy

Human hTREM2-DAP12 HEK293 cells were plated at 35,000 cells/well on 96-well poly-D-Lysine Biocoat plates (Corning) and allowed to adhere for 24 h. Cells were then serum-starved by media exchange to DMEM for 1 h, then antibody was spiked to achieve a final concentration of 10 nM and cells incubated at 37° C. for 10 min. For experiments including transferrin labeling, a Alexa647-transferrin conjugate (Invitrogen, T23366) was spiked just prior to antibody addition to achieve a final concentration of 20 ug/mL. Subsequently, the antibody-containing media was removed, and cells were fixed for 10 min with 4% paraformaldehyde in PBS. Cells were blocked and permeabilized for 30 min in 0.3% Triton/5% BSA in PBS, then stained overnight at 4° C. with 1:250 of rabbit anti-pSyk Tyr525/526 (Cell Signaling, #2711) and mouse anti-EEA1 (BD Biosciences, 610456, excluded for experiments using Alexa647-transferrin) in 0.06% Triton/1% BSA in PBS. Cells were then washed 3× with PBS and stained at 1:500 with Alexa 488 anti-human IgG (Jackson 109-545-003), Alexa 568 anti-rabbit (ThermoFisher, A10042), and Alexa 647 anti-mouse (ThermoFisher, A31572) in 0.06% Triton/1% BSA in PBS for 45 min at room temperature. Cells were stained with 1:1000 dilution of 1 mg/mL DAPI (ThermoFisher, 62248) in PBS for 10 min, then washed 2× with PBS and imaged using an Opera Phoenix High Content Imager (Perkin Elmer) at 63× magnification. Quantification of images was performed using the Harmony software (Perkin Elmer) using a spot identification algorithm to quantify the fluorescence intensity of spots identified in each channel. A masking algorithm was implemented in the Harmony software to quantify the percent of IgG or pSyk spots localized within EEA1-positive spots (early endosomes) or Tf-positive spots (recycling endosomes).

### Example 3. Characterization of Anti-TREM2 MV Antibodies Having Alternative Formats for TfR Binding

Anti-TREM2 molecules that bind to a TfR using different formats were evaluated based on their effects on sSYK signaling. In particular, bivalent (BV) and monovalent (MV) anti-TREM2 antibodies were compared, where the antibodies comprised either 1) a modified Fc polypeptide that specifically binds to a TfR; or 2) an scFab fragment that specifically binds to a TfR, wherein the scFab fragment was bound to the C-terminus of an Fc polypeptide (see, FIGS. 1A-1B).

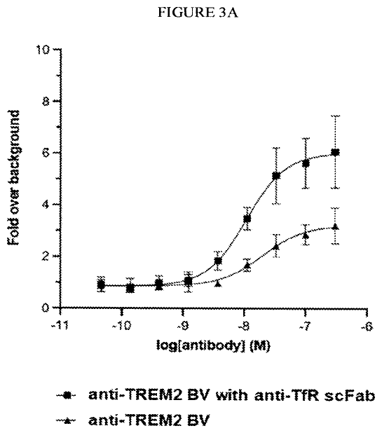

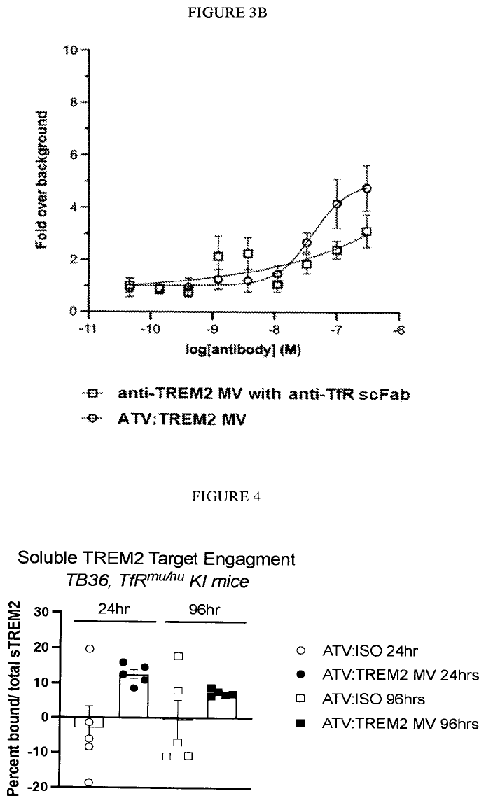

As shown in FIG. 3A and Table 8, hTREM2 pSYK signaling was evaluated with the following bivalent molecules: anti-TREM2 BV linked to an anti-TfR scFab and anti-TREM2 BV (non-TfR binding). These data indicate that TfR binding enhances hTREM2 pSYK signaling produced by the anti-TREM2 antibodies.

The effects of two monovalent anti-TREM2 antibodies on pSYK signaling were also investigated (FIG. 3B; Table 6). Specifically, an anti-TREM2 antibody linked to an anti-TfR scFab and an ATV:TREM2 MV antibody were compared. This evaluation demonstrated that TfR-binding could enhance the activity of a monovalent anti-TREM2 antibody, and this enhancement can be obtained with different TfR binding region formats.

TABLE 6
hTREM2 pSYK Signaling

| Molecules | EC50 (nM) | E Max |
|---|---|---|
| anti-TREM2 BV with anti-TfR scFab | 10.9 | 6.0 |
| anti-TREM2 BV | 19.8 | 3.2 |
| ATV:TREM2 MV | 40.7 | 5.0 |
| anti-TREM2 MV with anti-TfR scFab | >300 | ND |

### Materials and Methods

The recombinant antibodies were generated and expressed using methods similar to those described in Example 1. The light chain, heavy chain and Fc sequences for the various formats are set forth below in Table 7.

TABLE 7
Anti-TREM2 and ATV:TREM2 Sequences

| | Heavy Chain, | | |
|---|---|---|---|
| | optionally linked to | Second Heavy Chain | |
| Antibody | scFab | or Fc Polypeptide | Light Chain |
| anti-TREM2 BV | SEQ ID NO: 83 | SEQ ID NO: 83 | SEQ ID NO: 54 |
| anti-TREM2 BV with anti- | SEQ ID NO: 171 | SEQ ID NO: 142 or | SEQ ID NO: 54 |
| TfR scFab | | 165 | |
| ATV:TREM2 MV | SEQ ID NO: 94 or 106 | SEQ ID NO: 88 or 100 | SEQ ID NO: 54 |
| anti-TREM2 MV with anti- | SEQ ID NO: 171 | SEQ ID NO: 141 or | SEQ ID NO: 54 |
| TfR scFab | | 164 | |

The heavy chain and/or Fc polypeptide, including anti-TREM2 (BV), may be further processed during cell culture production, such that the C-terminal Lys residue is removed (i.e., the Lys residue at position 447, according to the EU numbering scheme). For example, as used herein, the term ATV:TREM2 MV may be used may be used to refer to monovalent antibodies having unprocessed sequences (i.e., SEQ ID NOs:94 and 88); monovalent antibodies comprising one or more processed sequences (i.e., selected from SEQ ID NOs: 106 and 100); or to a composition comprising a mixture of processed and unprocessed monovalent antibodies.

The hTREM2 pSYK signaling assays were performed using similar methods and materials as described in Example 2.

### Example 4. Characterization of Anti-TREM2 MV Antibody in Mouse Expressing Human TREM2

To assess the effect of the ATV:TREM2 MV antibody on microglia response, mice expressing human TREM2 and human/mouse chimeric TfR (TB36; TfR^{mu/hu} KI mice) were dosed with ATV:TREM2 MV (6.56 mg/kg), ATV:TREM2 (10 mg/kg), and isotype control ATV:ISO (10 mg/kg) via IV injection.

Bound and total sTREM2 levels in brain lysates from the mice were evaluated. ATV:TREM2 MV administration resulted in increased bound/total sTREM2 ratio in the brains as compared to the isotype control, thus demonstrating target engagement of ATV:TREM2 MV (FIG. 4; Table 8).

TABLE 8
Soluble TREM2 Target Engagement Percent
bound/total sTREM2 Mean (SEM)

| Timepoint | ATV:ISO | ATV:TREM2 | ATV:TREM2 MV |
|---|---|---|---|
| 24 h | −3.015 (6.327) | 63.19 (7.124) | 12.51 (1.298) |
| 96 h | −0.6816 (5.751) | 27.28 (3.405) | 7.266 (0.4324) |

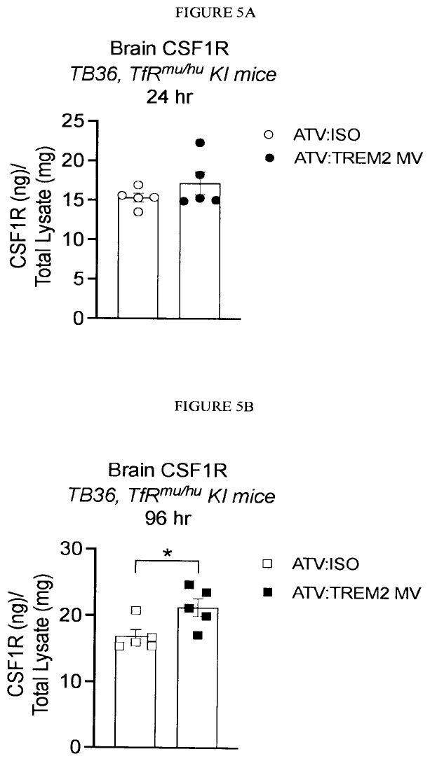

Additionally, ATV:TREM2 MV was found to elevate CSF1R and two cytokines (IP-10 and MCP-5) in vivo. CSF1R is expressed on microglia and is essential for survival. Therefore, CSF1R may be used as a potential pharmacodynamic biomarker to reflect microglia response. ATV:TREM2 MV was found to increase CSF1R 96 hr post dose but not 24 hr post dose; the effect of ATV:TREM2 MV on CSF1R appears to be milder compared to that of ATV:TREM2 (FIG. 5A-5B; Table 9).

TABLE 9
Brain CSF1R (ng)/Total Lysate (mg) Mean (SEM)

| Timepoint | ATV:ISO | ATV:TREM2 | ATV:TREM2 MV |
|---|---|---|---|
| 24 h | 15.32 (0.5401) | 23.86 (1.244) | 17.13 (1.429) |
| 96 h | 16.83 (1.008) | 31.97 (1.200) | 21.27 (1.340) |

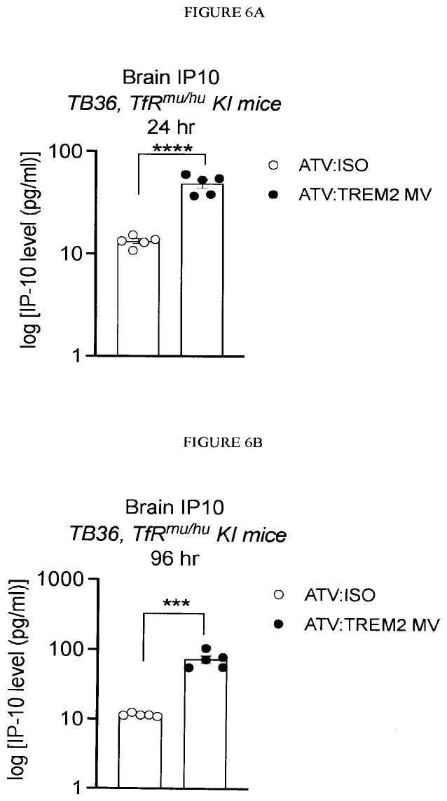

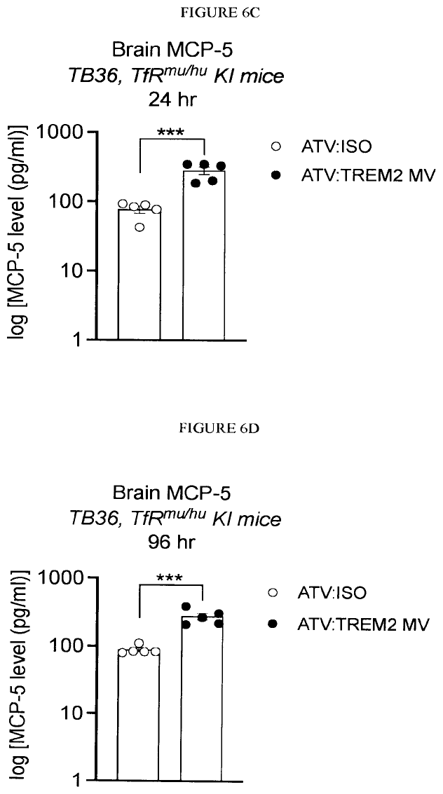

ATV:TREM2 MV was also found to significantly increase IP-10 at 24 hr and 96 hr post single dose as compared to the isotype control, but the effect of ATV:TREM2 MV was milder as compared to ATV:TREM2 (FIG. 6A-6B; Table 10). Similarly, ATV:TREM2 MV also significantly increased MCP-5 at 24 hr and 96 hr post single dose as compared to the isotype control, but the effect of ATV:TREM2 MV on cytokine elevation as compared to ATV:TREM2 was milder (FIG. 6C-6D; Table 11). Of note, ATV:TREM2 MV showed a similar magnitude increase in the cytokines at both 24 hr and 96 hr post single dose as compared to ATV:ISO, whereas ATV:TREM2 BV resulted in a cytokine spike at 24 hours post single dose.

TABLE 10
Log (IP10 level (pg/mL)) Mean (SEM)

| Timepoint | ATV:ISO | ATV:TREM2 | ATV:TREM2 MV |
|---|---|---|---|
| 24 h | 13.15 (0.7058) | 1262 (296.1) | 48.17 (4.563) |
| 96 h | 11.55 (0.2524) | 121.6 (13.42) | 74.03 (9.068) |

TABLE 11
Log (MCP-5 level (pg/mL)) Mean (SEM)

| Timepoint | ATV:ISO | ATV:TREM2 | ATV:TREM2 MV |
|---|---|---|---|
| 24 h | 77.13 (9.033) | 3378 (654.9) | 282.3 (36.37) |
| 96 h | 87.64 (5.686) | 429.3 (38.59) | 276.1 (32.34) |

These in vivo biomarker induction results are consistent with the in vitro data presented in the previous Examples above and demonstrate that TfR-binding can surprisingly confer activity to inactive forms of anti-TREM2 MV Fabs. As noted above, ATV:TREM2 MV resulted in a milder cytokine response as compared to ATV:TREM2, which may suggest the possibility that the lower activity of the MV format could potentially broaden the window between efficacy and safety. Here, the TfR binding acts to improve biodistribution in the brain, while also conferring agonist activity of the monovalent Fab.

### Materials and Methods

### Recombinant Antibodies

The recombinant antibodies (ATV:TREM2 (BV), ATV:TREM2 MV, and an isotype control) were generated and expressed using methods similar to those described in Example 1.

### Generation of TB36, TfR^{mu/hu} KI Mice

A human Trem2 BAC transgenic (tg) mouse model was generated by introduction of engineered BAC DNA CTD-2210D2 into the pronucleus of fertilized mouse eggs from C57BL/6J mice. The engineered BAC DNA CTD-2210D2 clone covers the entire human Trem2 coding region and its regulatory elements with deletion of the exon 1 from TREML1 and exon 3 from TREML2 to abolish the expression of TREML1 and TREML2. Human Trem2 BAC tg mice were backcrossed to C57BL/6J mice for three rounds and maintained as hemizygous. TfR^{mu/hu}KI mice were generated as described in International Patent Publication No. WO 2018/152285 using CRISPR/Cas9 technology to express human Tfrc apical domain within the murine Tfrc gene; the resulting chimeric TfR was expressed in vivo under the control of the endogenous promoter. The Human Trem2 BAC tg mice were further bred with the TfR^{mu/hu}KI mice to generate TB36, TfR^{mu/hu}KI mice for in vivo studies. Animals were housed in standard conditions with ad libitum access to food and water.

### Sample Preparation and Evaluation

Wild type mice expressing human TREM2 and human/mouse chimeric TfR (TB36; TfR^{mu/hu} KI mice) were dosed with ATV:TREM2 MV (6.56 mg/kg), ATV:TREM2 (10 mg/kg), and isotype control ATV:ISO (10 mg/kg) via IV injection. The doses were selected to ensure similar molar concentrations across the molecules. Mice were terminated 24 hours or 96 hours post a single dose and brains were dissected after PBS perfusion for target engagement and biomarker analysis.

The brains were homogenized in Cell Signaling lysis buffer (cat #9803) and supernatants were used for biochemical analysis. Bound and total sTREM2 levels in brain lysates were evaluated by using the following method. 96 well MSD small spot streptavidin plates (MSD—Meso Scale Discovery) were blocked with MSD Blocker-A (MSD) for 1 hour at RT. Plates were then washed 3× with TBST, then coated with biotinylated anti-TREM2 polyclonal antibody (R&D Systems BAF1828 for human, BAF1729 for mouse) at 1 μg/mL at RT for 1 hour. Standards (recombinant human or mouse TREM2 ECD prepared in house) were prepared in Assay Buffer (25% MSD Blocker-A in TBST) and serially diluted 1:4 in Assay Buffer. For cell-based samples, samples and standards were heated to 95 C for 5 min in an SDS-containing buffer. Samples were diluted 1:20 for mouse CSF and 1:10 for cell supernatant. Plates were washed 3× with TBST, then 30 μL of the samples or standards were added to the plates and incubated for one hour. Subsequently, for mouse samples, primary antibody 4D9 at 110 ug/mL prepared in house was directly added to the plate (3.3 μL/well), and incubated another hour at RT. Then, plates were washed 3× with TBST, and the primary detection antibody, sulfo-tagged goat anti-human TREM2 (R&D Systems AF1828 sulfo-tagged as per the MSD Gold Sulfo-tag NHS-ester kit protocol (MesoScale, R3122-A) or sulfo-tagged goat anti-human IgG (MSD, R32AJ-1) for mouse TREM2 detection, was diluted to 0.5 μg/mL in Assay Buffer, added to the plates, and incubated for one hour at room temperature. After washing with TBST, the MSD plates were developed using 2× MSD read buffer T, followed by detection using an MSD Sector plate reader (Methodical Mind, V1.0.38). MSD values were converted to absolute concentrations of TREM2.

CSF1R levels in brain homogenates were measured by a commercial ELISA assay (Abcam, ab240681) after a 40-fold dilution. Cytokines and chemokines, including IP-10 and MCP-5, were also measured in brain lysates using the Mouse Cytokine Array/Chemokine Array 44-Plex (MD44; Eve Technologies (Canada)).

TABLE 12
Informal Sequence Listing

| SEQ ID | | |
|---|---|---|
| NO | Sequence | Description |
| 1 | MEPLRLLILLFVTELSGAHNTTVFQGVAGQSLQVSCPYDSMKH | Human TREM2 protein |
| | WGRRKAWCRQLGEKGPCQRVVSTHNLWLLSFLRRWNGSTAIT | |
| | DDTLGGTLTITLRNLQPHDAGLYQCQSLHGSEADTLRKVL VEVL | |
| | ADPLDHRDAGDLWFPGESESFEDAHVEHSISRSLLEGEIPFPPTSI | |
| | LLLLACIFLIKILAASALWAAAWHGQKPGTHPPSELDCGHDPGY | |
| | QLQTLPGLRDT | |
| 2 | EVKLLDSGGGLVQAGGSLRLSCAGSGFTFTDFYMSWIRQPPGKA | CL0020306 V_{H} |
| | PEWLGVIRNKANGYTAGYNPSVKGRFTISRDNTQNILYLQMNTL | |
| | RAEDTAIYYCARLSYGFDYWGQGVMVTVSS | |
| 3 | DIVMTQGALPNPVPSGESASITCQSSKSLLHSNGKTYLNWYLQR | CL0020306 V_{L} |
| | PGQSPQLLIYWMSTRASGVSDRFSGSGSGTDFTLKISSVEAEDVG | |
| | VYYCQQFLEFPFTFGSGTKLEIK | |
| 4 | GFTFTDFYMS | CL0020306 CDR-H1; |
| | | CL0020164 CDR-H1; CDR-H1 |
| | | for CL0020188 and variants |
| | | CL0020188-1, CL0020188-2, |
| | | CL0020188-3, CL0020188-4, |
| | | CL0020188-5, CL0020188-6, |
| | | CL0020188-7, and |
| | | CL0020188-8 |
| 5 | VIRNKANGYTAGYNPSVKG | CL0020306 CDR-H2; CDR-H2 |
| | | for CL0020188 and variants |
| | | CL0020188-1, CL0020188-2, |
| | | CL0020188-3, and |
| | | CL0020188-4 |
| 6 | ARLSYGFDY | CL0020306 CDR-H3 |
| 7 | QSSKSLLHSNGKTYLN | CL0020306 CDR-L1; |
| | | CL0020164 CDR-L1; |
| | | CL0020307 CDR-L1; |
| | | CL0020307-1 CDR-L1; |
| | | CDR-L1 for CL0020188 and |
| | | variants CL0020188-1, |
| | | CL0020188-2, CL0020188-5, |
| | | and CL0020188-6 |
| 8 | WMSTRAS | CL0020306 CDR-L2; |
| | | CL0020307 CDR-L2; |
| | | CL0020307-1 CDR-L2; |
| | | CL0020164 CDR-L2; CDR-L2 |
| | | for CL0020188 and variants |
| | | CL0020188-1, CL0020188-2, |
| | | CL0020188-3, CL0020188-4, |
| | | CL0020188-5, CL0020188-6, |
| | | CL0020188-7, and |
| | | CL0020188-8 |
| 9 | QQFLEFPFT | CL0020306 CDR-L3; |
| | | CL0020307 CDR-L3; |
| | | CL0020307-1 CDR-L3 |
| 10 | EVKLLESGGGLVQPGGSLRLSCAASGFTFTNFYMSWIRQPPGRA | CL0020307 V_{H} |
| | PEWLGVIRNRPNGYTTDYNPSVKGRFTISRDNTQNILYLQMSTL | |
| | RADDTAFYYCTRLTYGFDYWGQGVMVTVSS | |
| 11 | DIVMTQGALPNPVPSGESASITCQSSKSLLHSNGKTYLNWYLQR | CL0020307 V_{L} |
| | PGQSPQLLIYWMSTRASGVSDRFSGSGSGTDFTLKISSVEAEVVG | |
| | VYYCQQFLEFPFTFGSGTKLEIK | |
| 12 | GFTFTNFYMS | CL0020307 CDR-H1 |
| 13 | VIRNRPNGYTTDYNPSVKG | CL0020307 CDR-H2 |
| 14 | TRLTYGFDY | CL0020307 CDR-H3 |
| 15 | EVKLLDSGGGLVQAGGSLRLSCAGSGFTFTDFYMSWIRQPPGKA | CL0020188 V_{H} |
| | PEWLGVIRNKANGYTAGYNPSVKGRFTISRDNTQNILYLQMNTL | |
| | RAEDTAIYYCARLTYGFDYWGQGVMVTVSS | |
| 16 | DIVMTQGALPNPVPSGESASITCQSSKSLLHSNGKTYLNWYLQR | CL0020188 V_{L} |
| | PGQSPQLLIYWMSTRASGVSDRFSGSGSGTDFTLKISSVEAEDVG | |
| | VYYCQQFLEYPFTFGSGTKLEIK | |
| 17 | ARLTYGFDY | CDR-H3 for CL0020188 and |
| | | variants CL0020188-1, |
| | | CL0020188-2, CL0020188-3, |
| | | CL0020188-4, CL0020188-5, |
| | | CL0020188-6, CL0020188-7, |
| | | and CL0020188-8; CL0020164 |
| | | CDR-H3 |
| 18 | QQFLEYPFT | CDR-L3 for CL0020188 and |
| | | variants CL0020188-1, |
| | | CL0020188-2, CL0020188-3, |
| | | CL0020188-4, CL0020188-5, |
| | | CL0020188-6, CL0020188-7, |
| | | and CL0020188-8 |
| 19 | EVQLVESGGGLVQPGGSLRLSCAASGFTFTDFYMSWVRQAPGK | CL0020188-1 V_{H}; |
| | GLEWVSVIRNKANGYTAGYNPSVKGRFTISRDNSKNTLYLQMN | CL0020188-3 V_{H} |
| | SLRAEDTAVYYCARLTYGFDYWGQGTLVTVSS | |
| 20 | DIVMTQTPLSLPVTPGEPASISCQSSKSLLHSNGKTYLNWYLQKP | CL0020188-1 V_{L}; CL0020188- |
| | GQSPQLLIYWMSTRASGVPDRFSGSGSGTDFTLKISRVEAEDVG | 2 V_{L}; |
| | VYYCQQFLEYPFTFGQGTKVEIK | CL0020188-5 V_{L}; |
| | | CL0020188-6 V_{L} |
| 21 | EVQLVESGGGLVQPGGSLRLSCAGSGFTFTDFYMSWVRQAPGK | CL0020188-2 V_{H}; |
| | GLEWVSVIRNKANGYTAGYNPSVKGRFTISRDNSKNTLYLQMN | CL0020188-4 V_{H} |
| | SLRAEDTAVYYCARLTYGFDYWGQGTLVTVSS | |
| 22 | DIVMTQTPLSLPVTPGEPASISCQSSKSLLHSTGKTYLNWYLQKP | CL0020188-3 V_{L}; |
| | GQSPQLLIYWMSTRASGVPDRFSGSGSGTDFTLKISRVEAEDVG | CL0020188-4 V_{L}; |
| | VYYCQQFLEYPFTFGQGTKVEIK | CL0020188-7 V_{L}; |
| | | CL0020188-8 V_{L} |
| 23 | QSSKSLLHSTGKTYLN | CDR-L1 for variants |
| | | CL0020188-3, CL0020188-4, |
| | | CL0020188-7, and |
| | | CL0020188-8 |
| 24 | EVQLVESGGGLVQPGGSLRLSCAASGFTFTDFYMSWVRQAPGK | CL0020188-5 V_{H}; |
| | GLEWVSVIRNKANAYTAGYNPSVKGRFTISRDNSKNTLYLQMN | CL0020188-7 V_{H} |
| | SLRAEDTAVYYCARLTYGFDYWGQGTLVTVSS | |
| 25 | VIRNKANAYTAGYNPSVKG | CDR-H2 for variants |
| | | CL0020188-5, CL0020188-6, |
| | | CL0020188-7, and |
| | | CL0020188-8 |
| 26 | EVQLVESGGGLVQPGGSLRLSCAGSGFTFTDFYMSWVRQAPGK | CL0020188-6 V_{H}; |
| | GPEWLSVIRNKANAYTAGYNPSVKGRFTISRDNSKNTLYLQMN | CL0020188-8 V_{H} |
| | SLRAEDTAVYYCARLTYGFDYWGQGTLVTVSS | |
| 27 | DIVMTQSPDSLAVSLGERATINCQSSKSLLHSNGKTYLNWYQQK | CL0020307-1 V_{L} |
| | PGQPPKLLIYWMSTRASGVPDRFSGSGSGTDFTLTISSLQAEDVA | |
| | VYYCQQFLEFPFTFGQGTKVEIK | |
| 28 | G-F-T-F-T-α₆-F-Y-M-S, wherein α₆ is D or N | CDR-H1 consensus sequence |
| 29 | V-I-R-N-β5-β6-N-β8-Y-T-β11-12-Y-N-P-S-V-K-G, | CDR-H2 consensus sequence |
| | wherein β5 is K or R; β6 is A or P; β8 is G or | |
| | A; β11 is A or T; and β12 is G or D | |
| 30 | γ₁-R-L-γ₄-Y-G-F-D-Y, wherein γ₁ is A or T; and γ₄ | CDR-H3 consensus sequence |
| | is T or S | |
| 31 | Q-S-S-K-S-L-L-H-S-δ₁₀-G-K-T-Y-L-N, wherein δ₁₀ is | CDR-L1 consensus sequence |
| | N or T | |
| 32 | Q-Q-F-L-E-ϕ₆-P-F-T, wherein ϕ₆ is Y or F | CDR-L3 consensus sequence |
| 33 | 000 | |
| 34 | GGGGS | Linker sequence |
| 35 | HHHHHH | 6X-His tag |
| 36 | ASTKGPSVFPLAPSSKSTSGGTAALGCLVKDYFPEPVTVSWNSG | Heavy chain constant domain 1 |
| | ALTSGVHTFPAVLQSSGLYSLSSVVTVPSSSLGTQTYICNVNHKP | (CH1) with human IgG1 hinge |
| | SNTKVDKKVEPKSCDKTHTCPPCP | amino acid sequence |
| 37 | RTVAAPSVFIFPPSDEQLKSGTASVVCLLNNFYPREAKVQWKVD | light chain constant domain |
| | NALQSGNSQESVTEQDSKDSTYSLSSTLTLSKADYEKHKVYACE | (CL) |
| | VTHQGLSSPVTKSFNRGEC | |
| 38 | APELLGGPSVFLFPPKPKDTLMISRTPEVTCVVVDVSHEDPEVKF | Wild-type human Fc sequence |
| | NWYVDGVEVHNAKTKPREEQYNSTYRVVSVLTVLHQDWLNG | positions 231-447 EU index |
| | KEYKCKVSNKALPAPIEKTISKAKGQPREPQVYTLPPSRDELTKN | numbering |
| | QVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| 39 | APEAAGGPSVFLFPPKPKDTLMISRTPEVTCVVVDVSHEDPEVKF | Fc polypeptide with hole, |
| | NWYVDGVEVHNAKTKPREEQYNSTYRVVSVLTVLHQDWLNG | LALA, and LS mutations |
| | KEYKCKVSNKALPAPIEKTISKAKGQPREPQVYTLPPSRDELTKN | |
| | QVSLSCAVKGFYPSDIAVEWESNGQPENNYKTTPPVLDSDGSFF | |
| | LVSKLTVDKSRWQQGNVFSCSVLHEALHSHYTQKSLSLSPGK | |
| 40 | APELLGGPSVFLFPPKPKDTLMISRTPEVTCVVVDVSHEDPEVKF | Clone 35.21 |
| | NWYVDGVEVHNAKTKPREEQYNSTYRVVSVLTVLHQDWLNG | |
| | KEYKCKVSNKALPAPIEKTISKAKGQPREPQVYTLPPSRDELTKN | |
| | QVSLTCLVKGFYPSDIAVWWESYGTEWSSYKTTPPVLDSDGSFF | |
| | LYSKLTVTKEEWQQGFVFSCSVMHEALHNHYTQKSLSLSPGK | |
| 41 | APEAAGGPSVFLFPPKPKDTLMISRTPEVTCVVVDVSHEDPEVKF | Clone CH3C.35.21 Fc |
| | NWYVDGVEVHNAKTKPREEQYNSTYRVVSVLTVLHQDWLNG | polypeptide with knob, LALA, |
| | KEYKCKVSNKALPAPIEKTISKAKGQPREPQVYTLPPSRDELTKN | and LS mutations |
| | QVSLWCLVKGFYPSDIAVWWESYGTEWSSYKTTPPVLDSDGSF | |
| | FLYSKLTVTKEEWQQGFVFSCSVLHEALHSHYTQKSLSLSPGK | |
| 42 | EVQLVESGGGLVQPGGSLRLSCAASGFTFTDFYMSWVRQAPGK | anti-TREM2/CH35.21 heavy |
| | GLEWVSVIRNKANAYTAGYNPSVKGRFTISRDNSKNTLYLQMN | chain with knob, LALA and LS |
| | SLRAEDTAVYYCARLTYGFDYWGQGTLVTVSSASTKGPSVFPL | mutations |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLWCLVKGFYPSDIAVWWESYGTEWSS | |
| | YKTTPPVLDSDGSFFLYSKLTVTKEEWQQGFVFSCSVLHEALHS | |
| | HYTQKSLSLSPGK | |
| 43 | APELLGGPSVFLFPPKPKDTLMISRTPEVTCVVVDVSHEDPEVKF | Clone CH3C.35.23.1.1 |
| | NWYVDGVEVHNAKTKPREEQYNSTYRVVSVLTVLHQDWLNG | |
| | KEYKCKVSNKALPAPIEKTISKAKGQPREPQVYTLPPSRDELTKN | |
| | QVSLTCLVKGFYPSDIAVEWESFGTEWSNYKTTPPVLDSDGSFF | |
| | LYSKLTVSKEEWQQGFVFSCSVMHEALHNHYTQKSLSLSPGK | |
| 44 | APEAAGGPSVFLFPPKPKDTLMISRTPEVTCVVVDVSHEDPEVKF | Clone CH3C.35.23.1.1 Fc |
| | NWYVDGVEVHNAKTKPREEQYNSTYRVVSVLTVLHQDWLNG | polypeptide with knob, LALA, |
| | KEYKCKVSNKALPAPIEKTISKAKGQPREPQVYTLPPSRDELTKN | and LS mutations |
| | QVSLWCLVKGFYPSDIAVEWESFGTEWSNYKTTPPVLDSDGSFF | |
| | LYSKLTVSKEEWQQGFVFSCSVLHEALHSHYTQKSLSLSPGK | |
| 45 | EVQLVESGGGLVQPGGSLRLSCAASGFTFTDFYMSWVRQAPGK | anti-TREM2/CH35.23.1.1 |
| | GLEWVSVIRNKANAYTAGYNPSVKGRFTISRDNSKNTLYLQMN | heavy chain with knob, LALA |
| | SLRAEDTAVYYCARLTYGFDYWGQGTLVTVSSASTKGPSVFPL | and LS mutations |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLWCLVKGFYPSDIAVEWESFGTEWSNY | |
| | KTTPPVLDSDGSFFLYSKLTVSKEEWQQGFVFSCSVLHEALHSH | |
| | YTQKSLSLSPGK | |
| 46 | APELLGGPSVFLFPPKPKDTLMISRTPEVTCVVVDVSHEDPEVKF | Clone CH3C.35.23.3 |
| | NWYVDGVEVHNAKTKPREEQYNSTYRVVSVLTVLHQDWLNG | |
| | KEYKCKVSNKALPAPIEKTISKAKGQPREPQVYTLPPSRDELTKN | |
| | QVSLTCLVKGFYPSDIAVEWESYGTEWVNYKTTPPVLDSDGSFF | |
| | LYSKLTVTKEEWQQGFVFSCSVMHEALHNHYTQKSLSLSPGK | |
| 47 | APEAAGGPSVFLFPPKPKDTLMISRTPEVTCVVVDVSHEDPEVKF | CH3C.35.23.3 Fc polypeptide |
| | NWYVDGVEVHNAKTKPREEQYNSTYRVVSVLTVLHQDWLNG | with knob, LALA, and LS |
| | KEYKCKVSNKALPAPIEKTISKAKGQPREPQVYTLPPSRDELTKN | mutations |
| | QVSLWCLVKGFYPSDIAVEWESYGTEWVNYKTTPPVLDSDGSF | |
| | FLYSKLTVTKEEWQQGFVFSCSVLHEALHSHYTQKSLSLSPGK | |
| 48 | EVQLVESGGGLVQPGGSLRLSCAASGFTFTDFYMSWVRQAPGK | anti-TREM2/CH35.23.3 |
| | GLEWVSVIRNKANAYTAGYNPSVKGRFTISRDNSKNTLYLQMN | heavy chain with knob, LALA |
| | SLRAEDTAVYYCARLTYGFDYWGQGTLVTVSSASTKGPSVFPL | and LS mutations |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLWCLVKGFYPSDIAVEWESYGTEWVN | |
| | YKTTPPVLDSDGSFFLYSKLTVTKEEWQQGFVFSCSVLHEALHS | |
| | HYTQKSLSLSPGK | |
| 49 | APELLGGPSVFLFPPKPKDTLMISRTPEVTCVVVDVSHEDPEVKF | Clone CH3C.35.23.4 |
| | NWYVDGVEVHNAKTKPREEQYNSTYRVVSVLTVLHQDWLNG | |
| | KEYKCKVSNKALPAPIEKTISKAKGQPREPQVYTLPPSRDELTKN | |
| | QVSLTCLVKGFYPSDIAVEWESYGTEWSNYKTTPPVLDSDGSFF | |
| | LYSKLTVSKEEWQQGFVFSCSVMHEALHNHYTQKSLSLSPGK | |
| 50 | APEAAGGPSVFLFPPKPKDTLMISRTPEVTCVVVDVSHEDPEVKF | CH3C.35.23.4 Fc polypeptide |
| | NWYVDGVEVHNAKTKPREEQYNSTYRVVSVLTVLHQDWLNG | with knob, LALA, and LS |
| | KEYKCKVSNKALPAPIEKTISKAKGQPREPQVYTLPPSRDELTKN | mutations |
| | QVSLWCLVKGFYPSDIAVEWESYGTEWSNYKTTPPVLDSDGSFF | |
| | LYSKLTVSKEEWQQGFVFSCSVLHEALHSHYTQKSLSLSPGK | |
| 51 | EVQLVESGGGLVQPGGSLRLSCAASGFTFTDFYMSWVRQAPGK | anti-TREM2/CH35.23.4 |
| | GLEWVSVIRNKANAYTAGYNPSVKGRFTISRDNSKNTLYLQMN | heavy chain with knob, LALA |
| | SLRAEDTAVYYCARLTYGFDYWGQGTLVTVSSASTKGPSVFPL | and LS mutations |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLWCLVKGFYPSDIAVEWESYGTEWSN | |
| | YKTTPPVLDSDGSFFLYSKLTVSKEEWQQGFVFSCSVLHEALHS | |
| | HYTQKSLSLSPGK | |
| 52 | EVQLVESGGGLVQPGGSLRLSCAASGFTFTDFYMSWVRQAPGK | anti-TREM2 heavy chain with |
| | GLEWVSVIRNKANAYTAGYNPSVKGRFTISRDNSKNTLYLQMN | hole and LS mutations |
| | SLRAEDTAVYYCARLTYGFDYWGQGTLVTVSSASTKGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPELLGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLSCAVKGFYPSDIAVEWESNGQPENNY | |
| | KTTPPVLDSDGSFFLVSKLTVDKSRWQQGNVFSCSVLHEALHSH | |
| | YTQKSLSLSPGK | |
| 53 | EVQLVESGGGLVQPGGSLRLSCAASGFTFTDFYMSWVRQAPGK | anti-TREM2 heavy chain with |
| | GLEWVSVIRNKANAYTAGYNPSVKGRFTISRDNSKNTLYLQMN | hole, LALA and LS mutations |
| | SLRAEDTAVYYCARLTYGFDYWGQGTLVTVSSASTKGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLSCAVKGFYPSDIAVEWESNGQPENNY | |
| | KTTPPVLDSDGSFFLVSKLTVDKSRWQQGNVFSCSVLHEALHSH | |
| | YTQKSLSLSPGK | |
| 54 | DIVMTQTPLSLPVTPGEPASISCQSSKSLLHSTGKTYLNWYLQKP | anti-TREM2 light chain |
| | GQSPQLLIYWMSTRASGVPDRFSGSGSGTDFTLKISRVEAEDVG | |
| | VYYCQQFLEYPFTFGQGTKVEIKRTVAAPSVFIFPPSDEQLKSGT | |
| | ASVVCLLNNFYPREAKVQWKVDNALQSGNSQESVTEQDSKDST | |
| | YSLSSTLTLSKADYEKHKVYACEVTHQGLSSPVTKSFNRGEC | |
| 55 | NSVIIVDKNGRLVYLVENPGGYVAYSKAATVTGKLVHANFGTK | Human TfR apical domain |
| | KDFEDLYTPVNGSIVIVRAGKITFAEKVANAESLNAIGVLIYMDQ | |
| | TKFPIVNAELSFFGHAHLGTGDPYTPGFPSFNHTQFPPSRSSGLPN | |
| | IPVQTISRAAAEKLFGNMEGDCPSDWKTDSTCRMVTSESKNVKL | |
| | TVS | |
| 56 | EPKSCDKTHTCPPCP | Human IgG1 hinge amino acid |
| | | sequence |
| 57 | DKTHTCPPCP | Portion of human IgG1 hinge |
| | | sequence |
| 58 | YxTEWSS | CH3 motif (TfR-binding) |
| 59 | TxxExxxxF | CH3 motif (TfR-binding) |
| 60 | MMDQARSAFSNLFGGEPLSYTRFSLARQVDGDNSHVEMKLGV | Cyno TfR |
| | DEEENTDNNTKANGTKPKRCGGNICYGTIAVIIFFLIGFMIGYLG | |
| | YCKGVEPKTECERLAGTESPAREEPEEDFPAAPRLYWDDLKRKL | |
| | SEKLDTTDFTSTIKLLNENLYVPREAGSQKDENLALYIENQFREF | |
| | KLSKVWRDQHFVKIQVKDSAQNSVIIVDKNGGLVYLVENPGGY | |
| | VAYSKAATVTGKLVHANFGTKKDFEDLDSPVNGSIVIVRAGKIT | |
| | FAEKVANAESLNAIGVLIYMDQTKFPIVKADLSFFGHAHLGTGD | |
| | PYTPGFPSFNHTQFPPSQSSGLPNIPVQTISRAAAEKLFGNMEGDC | |
| | PSDWKTDSTCKMVTSENKSVKLTVSNVLKETKILNIFGVIKGFV | |
| | EPDHYVVVGAQRDAWGPGAAKSSVGTALLLKLAQMFSDMVLK | |
| | DGFQPSRSIIFASWSAGDFGSVGATEWLEGYLSSLHLKAFTYINL | |
| | DKAVLGTSNFKVSASPLLYTLIEKTMQDVKHPVTGRSLYQDSN | |
| | WASKVEKLTLDNAAFPFLAYSGIPAVSFCFCEDTDYPYLGTTMD | |
| | TYKELVERIPELNKVARAAAEVAGQFVIKLTHDTELNLDYERYN | |
| | SQLLLFLRDLNQYRADVKEMGLSLQWLYSARGDFFRATSRLTT | |
| | DFRNAEKRDKFVMKKLNDRVMRVEYYFLSPYVSPKESPFRHVF | |
| | WGSGSHTLSALLESLKLRRQNNSAFNETLFRNQLALATWTIQGA | |
| | ANALSGDVWDIDNEF | |
| 61 | APELLGGPSVFLFPPKPKDTLMISRTPEVTCVVVDVSHEDPEVKF | Fc polypeptide with hole and |
| | NWYVDGVEVHNAKTKPREEQYNSTYRVVSVLTVLHQDWLNG | LS mutations |
| | KEYKCKVSNKALPAPIEKTISKAKGQPREPQVYTLPPSRDELTKN | |
| | QVSLSCAVKGFYPSDIAVEWESNGQPENNYKTTPPVLDSDGSFF | |
| | LVSKLTVDKSRWQQGNVFSCSVLHEALHSHYTQKSLSLSPGK | |
| 62 | MMDQARSAFSNLFGGEPLSYTRFSLARQVDGDNSHVEMKLAV | Human transferrin receptor |
| | DEEENADNNTKANVTKPKRCSGSICYGTIAVIVFFLIGFMIGYLG | protein 1 (TFR1) |
| | YCKGVEPKTECERLAGTESPVREEPGEDFPAARRLYWDDLKRK | |
| | LSEKLDSTDFTGTIKLLNENSYVPREAGSQKDENLALYVENQFR | |
| | EFKLSKVWRDQHFVKIQVKDSAQNSVIIVDKNGRLVYLVENPG | |
| | GYVAYSKAATVTGKLVHANFGTKKDFEDLYTPVNGSIVIVRAG | |
| | KITFAEKVANAESLNAIGVLIYMDQTKFPIVNAELSFFGHAHLGT | |
| | GDPYTPGFPSFNHTQFPPSRSSGLPNIPVQTISRAAAEKLFGNMEG | |
| | DCPSDWKTDSTCRMVTSESKNVKLTVSNVLKEIKILNIFGVIKGF | |
| | VEPDHYVVVGAQRDAWGPGAAKSGVGTALLLKLAQMFSDMV | |
| | LKDGFQPSRSIIFASWSAGDFGSVGATEWLEGYLSSLHLKAFTYI | |
| | NLDKAVLGTSNFKVSASPLLYTLIEKTMQNVKHPVTGQFLYQDS | |
| | NWASKVEKLTLDNAAFPFLAYSGIPAVSFCFCEDTDYPYLGTTM | |
| | DTYKELIERIPELNKVARAAAEVAGQFVIKLTHDVELNLDYERY | |
| | NSQLLSFVRDLNQYRADIKEMGLSLQWLYSARGDFFRATSRLTT | |
| | DFGNAEKTDRFVMKKLNDRVMRVEYHFLSPYVSPKESPFRHVF | |
| | WGSGSHTLPALLENLKLRKQNNGAFNETLFRNQLALATWTIQG | |
| | AANALSGDVWDIDNEF | |
| 63 | APEAAGGPSVFLFPPKPKDTLMISRTPEVTCVVVDVSHEDPEVKF | Fc polypeptide with hole, |
| | NWYVDGVEVHNAKTKPREEQYNSTYRVVSVLTVLHQDWLNG | LALA, and LS mutations, |
| | KEYKCKVSNKALPAPIEKTISKAKGQPREPQVYTLPPSRDELTKN | truncated |
| | QVSLSCAVKGFYPSDIAVEWESNGQPENNYKTTPPVLDSDGSFF | |
| | LVSKLTVDKSRWQQGNVFSCSVLHEALHSHYTQKSLSLSPG | |
| 64 | APEAAGGPSVFLFPPKPKDTLMISRTPEVTCVVVDVSHEDPEVKF | Clone CH3C.35.21 Fc |
| | NWYVDGVEVHNAKTKPREEQYNSTYRVVSVLTVLHQDWLNG | polypeptide with knob, LALA, |
| | KEYKCKVSNKALPAPIEKTISKAKGQPREPQVYTLPPSRDELTKN | and LS mutations, truncated |
| | QVSLWCLVKGFYPSDIAVWWESYGTEWSSYKTTPPVLDSDGSF | |
| | FLYSKLTVTKEEWQQGFVFSCSVLHEALHSHYTQKSLSLSPG | |
| 65 | EVQLVESGGGLVQPGGSLRLSCAASGFTFTDFYMSWVRQAPGK | anti-TREM2/CH35.21 heavy |
| | GLEWVSVIRNKANAYTAGYNPSVKGRFTISRDNSKNTLYLQMN | chain with knob, LALA and LS |
| | SLRAEDTAVYYCARLTYGFDYWGQGTLVTVSSASTKGPSVFPL | mutations, truncated |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLWCLVKGFYPSDIAVWWESYGTEWSS | |
| | YKTTPPVLDSDGSFFLYSKLTVTKEEWQQGFVFSCSVLHEALHS | |
| | HYTQKSLSLSPG | |
| 66 | APEAAGGPSVFLFPPKPKDTLMISRTPEVTCVVVDVSHEDPEVKF | Clone CH3C.35.23.1.1 Fc |
| | NWYVDGVEVHNAKTKPREEQYNSTYRVVSVLTVLHQDWLNG | polypeptide with knob, LALA, |
| | KEYKCKVSNKALPAPIEKTISKAKGQPREPQVYTLPPSRDELTKN | and LS mutations, truncated |
| | QVSLWCLVKGFYPSDIAVEWESFGTEWSNYKTTPPVLDSDGSFF | |
| | LYSKLTVSKEEWQQGFVFSCSVLHEALHSHYTQKSLSLSPG | |
| 67 | EVQLVESGGGLVQPGGSLRLSCAASGFTFTDFYMSWVRQAPGK | anti-TREM2/CH35.23.1.1 |
| | GLEWVSVIRNKANAYTAGYNPSVKGRFTISRDNSKNTLYLQMN | heavy chain with knob, LALA |
| | SLRAEDTAVYYCARLTYGFDYWGQGTLVTVSSASTKGPSVFPL | and LS mutations, truncated |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLWCLVKGFYPSDIAVEWESFGTEWSNY | |
| | KTTPPVLDSDGSFFLYSKLTVSKEEWQQGFVFSCSVLHEALHSH | |
| | YTQKSLSLSPG | |
| 68 | APEAAGGPSVFLFPPKPKDTLMISRTPEVTCVVVDVSHEDPEVKF | CH3C.35.23.3 Fc polypeptide |
| | NWYVDGVEVHNAKTKPREEQYNSTYRVVSVLTVLHQDWLNG | with knob, LALA, and LS |
| | KEYKCKVSNKALPAPIEKTISKAKGQPREPQVYTLPPSRDELTKN | mutations, truncated |
| | QVSLWCLVKGFYPSDIAVEWESYGTEWVNYKTTPPVLDSDGSF | |
| | FLYSKLTVTKEEWQQGFVFSCSVLHEALHSHYTQKSLSLSPG | |
| 69 | EVQLVESGGGLVQPGGSLRLSCAASGFTFTDFYMSWVRQAPGK | anti-TREM2/CH35.23.3 |
| | GLEWVSVIRNKANAYTAGYNPSVKGRFTISRDNSKNTLYLQMN | heavy chain with knob, LALA |
| | SLRAEDTAVYYCARLTYGFDYWGQGTLVTVSSASTKGPSVFPL | and LS mutations, truncated |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLWCLVKGFYPSDIAVEWESYGTEWVN | |
| | YKTTPPVLDSDGSFFLYSKLTVTKEEWQQGFVFSCSVLHEALHS | |
| | HYTQKSLSLSPG | |
| 70 | APEAAGGPSVFLFPPKPKDTLMISRTPEVTCVVVDVSHEDPEVKF | CH3C.35.23.4 Fc polypeptide |
| | NWYVDGVEVHNAKTKPREEQYNSTYRVVSVLTVLHQDWLNG | with knob, LALA, and LS |
| | KEYKCKVSNKALPAPIEKTISKAKGQPREPQVYTLPPSRDELTKN | mutations, truncated |
| | QVSLWCLVKGFYPSDIAVEWESYGTEWSNYKTTPPVLDSDGSFF | |
| | LYSKLTVSKEEWQQGFVFSCSVLHEALHSHYTQKSLSLSPG | |
| 71 | EVQLVESGGGLVQPGGSLRLSCAASGFTFTDFYMSWVRQAPGK | anti-TREM2/CH35.23.4 |
| | GLEWVSVIRNKANAYTAGYNPSVKGRFTISRDNSKNTLYLQMN | heavy chain with knob, LALA |
| | SLRAEDTAVYYCARLTYGFDYWGQGTLVTVSSASTKGPSVFPL | and LS mutations, truncated |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLWCLVKGFYPSDIAVEWESYGTEWSN | |
| | YKTTPPVLDSDGSFFLYSKLTVSKEEWQQGFVFSCSVLHEALHS | |
| | HYTQKSLSLSPG | |
| 72 | EVQLVESGGGLVQPGGSLRLSCAASGFTFTDFYMSWVRQAPGK | anti-TREM2 heavy chain with |
| | GLEWVSVIRNKANAYTAGYNPSVKGRFTISRDNSKNTLYLQMN | hole and LS mutations, |
| | SLRAEDTAVYYCARLTYGFDYWGQGTLVTVSSASTKGPSVFPL | truncated |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPELLGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLSCAVKGFYPSDIAVEWESNGQPENNY | |
| | KTTPPVLDSDGSFFLVSKLTVDKSRWQQGNVFSCSVLHEALHSH | |
| | YTQKSLSLSPG | |
| 73 | EVQLVESGGGLVQPGGSLRLSCAASGFTFTDFYMSWVRQAPGK | anti-TREM2 heavy chain with |
| | GLEWVSVIRNKANAYTAGYNPSVKGRFTISRDNSKNTLYLQMN | hole, LALA and LS mutations, |
| | SLRAEDTAVYYCARLTYGFDYWGQGTLVTVSSASTKGPSVFPL | truncated |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLSCAVKGFYPSDIAVEWESNGQPENNY | |
| | KTTPPVLDSDGSFFLVSKLTVDKSRWQQGNVFSCSVLHEALHSH | |
| | YTQKSLSLSPG | |
| 83 | EVQLVESGGGLVQPGGSLRLSCAASGFTFTDFYMSWVRQAPGK | anti-TREM2 heavy chain with |
| | GLEWVSVIRNKANAYTAGYNPSVKGRFTISRDNSKNTLYLQMN | LALA mutations |
| | SLRAEDTAVYYCARLTYGFDYWGQGTLVTVSSASTKGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| 84 | APELLGGPSVFLFPPKPKDTLMISRTPEVTCVVVDVSHEDPEVKF | Fc polypeptide with hole and |
| | NWYVDGVEVHNAKTKPREEQYNSTYRVVSVLTVLHQDWLNG | LS mutations, truncated |
| | KEYKCKVSNKALPAPIEKTISKAKGQPREPQVYTLPPSRDELTKN | |
| | QVSLSCAVKGFYPSDIAVEWESNGQPENNYKTTPPVLDSDGSFF | |
| | LVSKLTVDKSRWQQGNVFSCSVLHEALHSHYTQKSLSLSPG | |
| 85 | DKTHTCPPCPAPELLGGPSVFLFPPKPKDTLMISRTPEVTCVVVD | Fc polypeptide with hole and |
| | VSHEDPEVKFNWYVDGVEVHNAKTKPREEQYNSTYRVVSVLT | LS mutations and portion of |
| | VLHQDWLNGKEYKCKVSNKALPAPIEKTISKAKGQPREPQVYT | human IgG1 hinge sequence |
| | LPPSRDELTKNQVSLSCAVKGFYPSDIAVEWESNGQPENNYKTT | |
| | PPVLDSDGSFFLVSKLTVDKSRWQQGNVFSCSVLHEALHSHYTQ | |
| | KSLSLSPGK | |
| 86 | DKTHTCPPCPAPEAAGGPSVFLFPPKPKDTLMISRTPEVTCVVVD | Fc polypeptide with hole, |
| | VSHEDPEVKFNWYVDGVEVHNAKTKPREEQYNSTYRVVSVLT | LALA, and LS mutations, and |
| | VLHQDWLNGKEYKCKVSNKALPAPIEKTISKAKGQPREPQVYT | portion of human IgG1 hinge |
| | LPPSRDELTKNQVSLSCAVKGFYPSDIAVEWESNGQPENNYKTT | sequence |
| | PPVLDSDGSFFLVSKLTVDKSRWQQGNVFSCSVLHEALHSHYTQ | |
| | KSLSLSPGK | |
| 87 | APEAAGGPSVFLFPPKPKDTLMISRTPEVTCVVVDVSHEDPEVKF | Fc polypeptide with hole, |
| | NWYVDGVEVHNAKTKPREEQYGSTYRVVSVLTVLHQDWLNG | LALAPG, N297G and LS |
| | KEYKCKVSNKALGAPIEKTISKAKGQPREPQVYTLPPSRDELTK | mutations |
| | NQVSLSCAVKGFYPSDIAVEWESNGQPENNYKTTPPVLDSDGSF | |
| | FLVSKLTVDKSRWQQGNVFSCSVLHEALHSHYTQKSLSLSPGK | |
| 88 | DKTHTCPPCPAPEAAGGPSVFLFPPKPKDTLMISRTPEVTCVVVD | Fc polypeptide with hole, |
| | VSHEDPEVKFNWYVDGVEVHNAKTKPREEQYGSTYRVVSVLT | LALAPG, N297G and LS |
| | VLHQDWLNGKEYKCKVSNKALGAPIEKTISKAKGQPREPQVYT | mutations and portion of human |
| | LPPSRDELTKNQVSLSCAVKGFYPSDIAVEWESNGQPENNYKTT | IgG1 hinge |
| | PPVLDSDGSFFLVSKLTVDKSRWQQGNVFSCSVLHEALHSHYTQ | |
| | KSLSLSPGK | |
| 89 | APEAAGGPSVFLFPPKPKDTLMISRTPEVTCVVVDVSHEDPEVKF | Clone CH3C.35.21 Fc |
| | NWYVDGVEVHNAKTKPREEQYGSTYRVVSVLTVLHQDWLNG | polypeptide with knob, |
| | KEYKCKVSNKALGAPIEKTISKAKGQPREPQVYTLPPSRDELTK | LALAPG, N297G, and LS |
| | NQVSLWCLVKGFYPSDIAVWWESYGTEWSSYKTTPPVLDSDGS | mutations |
| | FFLYSKLTVTKEEWQQGFVFSCSVLHEALHSHYTQKSLSLSPGK | |
| 90 | EVQLVESGGGLVQPGGSLRLSCAASGFTFTDFYMSWVRQAPGK | anti-TREM2/CH35.21 heavy |
| | GLEWVSVIRNKANAYTAGYNPSVKGRFTISRDNSKNTLYLQMN | chain with knob, LALAPG, |
| | SLRAEDTAVYYCARLTYGFDYWGQGTLVTVSSASTKGPSVFPL | N297G, and LS mutations |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYGSTYRVVS | |
| | VLTVLHQDWLNGKEYKCKVSNKALGAPIEKTISKAKGQPREPQ | |
| | VYTLPPSRDELTKNQVSLWCLVKGFYPSDIAVWWESYGTEWSS | |
| | YKTTPPVLDSDGSFFLYSKLTVTKEEWQQGFVFSCSVLHEALHS | |
| | HYTQKSLSLSPGK | |
| 91 | APEAAGGPSVFLFPPKPKDTLMISRTPEVTCVVVDVSHEDPEVKF | Clone CH3C.35.23.1.1 Fc |
| | NWYVDGVEVHNAKTKPREEQYGSTYRVVSVLTVLHQDWLNG | polypeptide with knob, |
| | KEYKCKVSNKALGAPIEKTISKAKGQPREPQVYTLPPSRDELTK | LALAPG, N297G, and LS |
| | NQVSLWCLVKGFYPSDIAVEWESFGTEWSNYKTTPPVLDSDGSF | mutations |
| | FLYSKLTVSKEEWQQGFVFSCSVLHEALHSHYTQKSLSLSPGK | |
| 92 | EVQLVESGGGLVQPGGSLRLSCAASGFTFTDFYMSWVRQAPGK | anti-TREM2/CH35.23.1.1 |
| | GLEWVSVIRNKANAYTAGYNPSVKGRFTISRDNSKNTLYLQMN | heavy chain with knob, |
| | SLRAEDTAVYYCARLTYGFDYWGQGTLVTVSSASTKGPSVFPL | LALAPG, N297G, and LS |
| | APSSKSTSGGTAALGCLVKDYFPEPVTVSWNSGALTSGVHTFPA | mutations |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYGSTYRVVS | |
| | VLTVLHQDWLNGKEYKCKVSNKALGAPIEKTISKAKGQPREPQ | |
| | VYTLPPSRDELTKNQVSLWCLVKGFYPSDIAVEWESFGTEWSNY | |
| | KTTPPVLDSDGSFFLYSKLTVSKEEWQQGFVFSCSVLHEALHSH | |
| | YTQKSLSLSPGK | |
| 93 | APEAAGGPSVFLFPPKPKDTLMISRTPEVTCVVVDVSHEDPEVKF | CH3C.35.23.3 Fc polypeptide |
| | NWYVDGVEVHNAKTKPREEQYGSTYRVVSVLTVLHQDWLNG | with knob, LALAPG, N297G, |
| | KEYKCKVSNKALGAPIEKTISKAKGQPREPQVYTLPPSRDELTK | and LS mutations |
| | NQVSLWCLVKGFYPSDIAVEWESYGTEWVNYKTTPPVLDSDGS | |
| | FFLYSKLTVTKEEWQQGFVFSCSVLHEALHSHYTQKSLSLSPGK | |
| 94 | EVQLVESGGGLVQPGGSLRLSCAASGFTFTDFYMSWVRQAPGK | anti-TREM2/CH35.23.3 |
| | GLEWVSVIRNKANAYTAGYNPSVKGRFTISRDNSKNTLYLQMN | heavy chain with knob, |
| | SLRAEDTAVYYCARLTYGFDYWGQGTLVTVSSASTKGPSVFPL | LALAPG, N297G, and LS |
| | APSSKSTSGGTAALGCLVKDYFPEPVTVSWNSGALTSGVHTFPA | mutations |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYGSTYRVVS | |
| | VLTVLHQDWLNGKEYKCKVSNKALGAPIEKTISKAKGQPREPQ | |
| | VYTLPPSRDELTKNQVSLWCLVKGFYPSDIAVEWESYGTEWVN | |
| | YKTTPPVLDSDGSFFLYSKLTVTKEEWQQGFVFSCSVLHEALHS | |
| | HYTQKSLSLSPGK | |
| 95 | APEAAGGPSVFLFPPKPKDTLMISRTPEVTCVVVDVSHEDPEVKF | CH3C.35.23.4 Fc polypeptide |
| | NWYVDGVEVHNAKTKPREEQYGSTYRVVSVLTVLHQDWLNG | with knob, LALAPG, N297G, |
| | KEYKCKVSNKALGAPIEKTISKAKGQPREPQVYTLPPSRDELTK | and LS mutations |
| | NQVSLWCLVKGFYPSDIAVEWESYGTEWSNYKTTPPVLDSDGS | |
| | FFLYSKLTVSKEEWQQGFVFSCSVLHEALHSHYTQKSLSLSPGK | |
| 96 | EVQLVESGGGLVQPGGSLRLSCAASGFTFTDFYMSWVRQAPGK | anti-TREM2/CH35.23.4 |
| | GLEWVSVIRNKANAYTAGYNPSVKGRFTISRDNSKNTLYLQMN | heavy chain with knob, |
| | SLRAEDTAVYYCARLTYGFDYWGQGTLVTVSSASTKGPSVFPL | LALAPG, N297G, and LS |
| | APSSKSTSGGTAALGCLVKDYFPEPVTVSWNSGALTSGVHTFPA | mutations |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYGSTYRVVS | |
| | VLTVLHQDWLNGKEYKCKVSNKALGAPIEKTISKAKGQPREPQ | |
| | VYTLPPSRDELTKNQVSLWCLVKGFYPSDIAVEWESYGTEWSN | |
| | YKTTPPVLDSDGSFFLYSKLTVSKEEWQQGFVFSCSVLHEALHS | |
| | HYTQKSLSLSPGK | |
| 97 | DKTHTCPPCPAPELLGGPSVFLFPPKPKDTLMISRTPEVTCVVVD | Fc polypeptide with hole and |
| | VSHEDPEVKFNWYVDGVEVHNAKTKPREEQYNSTYRVVSVLT | LS mutations and portion of |
| | VLHQDWLNGKEYKCKVSNKALPAPIEKTISKAKGQPREPQVYT | human IgG1 hinge sequence, |
| | LPPSRDELTKNQVSLSCAVKGFYPSDIAVEWESNGQPENNYKTT | truncated |
| | PPVLDSDGSFFLVSKLTVDKSRWQQGNVFSCSVLHEALHSHYTQ | |
| | KSLSLSPG | |
| 98 | DKTHTCPPCPAPEAAGGPSVFLFPPKPKDTLMISRTPEVTCVVVD | Fc polypeptide with hole, |
| | VSHEDPEVKFNWYVDGVEVHNAKTKPREEQYNSTYRVVSVLT | LALA, and LS mutations, and |
| | VLHQDWLNGKEYKCKVSNKALPAPIEKTISKAKGQPREPQVYT | portion of human IgG1 hinge |
| | LPPSRDELTKNQVSLSCAVKGFYPSDIAVEWESNGQPENNYKTT | sequence, truncated |
| | PPVLDSDGSFFLVSKLTVDKSRWQQGNVFSCSVLHEALHSHYTQ | |
| | KSLSLSPG | |
| 99 | APEAAGGPSVFLFPPKPKDTLMISRTPEVTCVVVDVSHEDPEVKF | Fc polypeptide with hole, |
| | NWYVDGVEVHNAKTKPREEQYGSTYRVVSVLTVLHQDWLNG | LALAPG, N297G and LS |
| | KEYKCKVSNKALGAPIEKTISKAKGQPREPQVYTLPPSRDELTK | mutations, truncated |
| | NQVSLSCAVKGFYPSDIAVEWESNGQPENNYKTTPPVLDSDGSF | |
| | FLVSKLTVDKSRWQQGNVFSCSVLHEALHSHYTQKSLSLSPG | |
| 100 | DKTHTCPPCPAPEAAGGPSVFLFPPKPKDTLMISRTPEVTCVVVD | Fc polypeptide with hole, |
| | VSHEDPEVKFNWYVDGVEVHNAKTKPREEQYGSTYRVVSVLT | LALAPG, N297G and LS |
| | VLHQDWLNGKEYKCKVSNKALGAPIEKTISKAKGQPREPQVYT | mutations and portion of human |
| | LPPSRDELTKNQVSLSCAVKGFYPSDIAVEWESNGQPENNYKTT | IgG1 hinge, truncated |
| | PPVLDSDGSFFLVSKLTVDKSRWQQGNVFSCSVLHEALHSHYTQ | |
| | KSLSLSPG | |
| 101 | APEAAGGPSVFLFPPKPKDTLMISRTPEVTCVVVDVSHEDPEVKF | Clone CH3C.35.21 Fc |
| | NWYVDGVEVHNAKTKPREEQYGSTYRVVSVLTVLHQDWLNG | polypeptide with knob, |
| | KEYKCKVSNKALGAPIEKTISKAKGQPREPQVYTLPPSRDELTK | LALAPG, N297G, and LS |
| | NQVSLWCLVKGFYPSDIAVWWESYGTEWSSYKTTPPVLDSDGS | mutations, truncated |
| | FFLYSKLTVTKEEWQQGFVFSCSVLHEALHSHYTQKSLSLSPG | |
| 102 | EVQLVESGGGLVQPGGSLRLSCAASGFTFTDFYMSWVRQAPGK | anti-TREM2/CH35.21 heavy |
| | GLEWVSVIRNKANAYTAGYNPSVKGRFTISRDNSKNTLYLQMN | chain with knob, LALAPG, |
| | SLRAEDTAVYYCARLTYGFDYWGQGTLVTVSSASTKGPSVFPL | N297G, and LS mutations, |
| | APSSKSTSGGTAALGCLVKDYFPEPVTVSWNSGALTSGVHTFPA | truncated |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYGSTYRVVS | |
| | VLTVLHQDWLNGKEYKCKVSNKALGAPIEKTISKAKGQPREPQ | |
| | VYTLPPSRDELTKNQVSLWCLVKGFYPSDIAVWWESYGTEWSS | |
| | YKTTPPVLDSDGSFFLYSKLTVTKEEWQQGFVFSCSVLHEALHS | |
| | HYTQKSLSLSPG | |
| 103 | APEAAGGPSVFLFPPKPKDTLMISRTPEVTCVVVDVSHEDPEVKF | Clone CH3C.35.23.1.1 Fc |
| | NWYVDGVEVHNAKTKPREEQYGSTYRVVSVLTVLHQDWLNG | polypeptide with knob, |
| | KEYKCKVSNKALGAPIEKTISKAKGQPREPQVYTLPPSRDELTK | LALAPG, N297G, and LS |
| | NQVSLWCLVKGFYPSDIAVEWESFGTEWSNYKTTPPVLDSDGSF | mutations, truncated |
| | FLYSKLTVSKEEWQQGFVFSCSVLHEALHSHYTQKSLSLSPG | |
| 104 | EVQLVESGGGLVQPGGSLRLSCAASGFTFTDFYMSWVRQAPGK | anti-TREM2/CH35.23.1.1 |
| | GLEWVSVIRNKANAYTAGYNPSVKGRFTISRDNSKNTLYLQMN | heavy chain with knob, |
| | SLRAEDTAVYYCARLTYGFDYWGQGTLVTVSSASTKGPSVFPL | LALAPG, N297G, and LS |
| | APSSKSTSGGTAALGCLVKDYFPEPVTVSWNSGALTSGVHTFPA | mutations, truncated |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYGSTYRVVS | |
| | VLTVLHQDWLNGKEYKCKVSNKALGAPIEKTISKAKGQPREPQ | |
| | VYTLPPSRDELTKNQVSLWCLVKGFYPSDIAVEWESFGTEWSNY | |
| | KTTPPVLDSDGSFFLYSKLTVSKEEWQQGFVFSCSVLHEALHSH | |
| | YTQKSLSLSPG | |
| 105 | APEAAGGPSVFLFPPKPKDTLMISRTPEVTCVVVDVSHEDPEVKF | CH3C.35.23.3 Fc polypeptide |
| | NWYVDGVEVHNAKTKPREEQYGSTYRVVSVLTVLHQDWLNG | with knob, LALAPG, N297G, |
| | KEYKCKVSNKALGAPIEKTISKAKGQPREPQVYTLPPSRDELTK | and LS mutations, truncated |
| | NQVSLWCLVKGFYPSDIAVEWESYGTEWVNYKTTPPVLDSDGS | |
| | FFLYSKLTVTKEEWQQGFVFSCSVLHEALHSHYTQKSLSLSPG | |
| 106 | EVQLVESGGGLVQPGGSLRLSCAASGFTFTDFYMSWVRQAPGK | anti-TREM2/CH35.23.3 |
| | GLEWVSVIRNKANAYTAGYNPSVKGRFTISRDNSKNTLYLQMN | heavy chain with knob, |
| | SLRAEDTAVYYCARLTYGFDYWGQGTLVTVSSASTKGPSVFPL | LALAPG, N297G, and LS |
| | APSSKSTSGGTAALGCLVKDYFPEPVTVSWNSGALTSGVHTFPA | mutations, truncated |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYGSTYRVVS | |
| | VLTVLHQDWLNGKEYKCKVSNKALGAPIEKTISKAKGQPREPQ | |
| | VYTLPPSRDELTKNQVSLWCLVKGFYPSDIAVEWESYGTEWVN | |
| | YKTTPPVLDSDGSFFLYSKLTVTKEEWQQGFVFSCSVLHEALHS | |
| | HYTQKSLSLSPG | |
| 107 | APEAAGGPSVFLFPPKPKDTLMISRTPEVTCVVVDVSHEDPEVKF | CH3C.35.23.4 Fc polypeptide |
| | NWYVDGVEVHNAKTKPREEQYGSTYRVVSVLTVLHQDWLNG | with knob, LALAPG, N297G, |
| | KEYKCKVSNKALGAPIEKTISKAKGQPREPQVYTLPPSRDELTK | and LS mutations, truncated |
| | NQVSLWCLVKGFYPSDIAVEWESYGTEWSNYKTTPPVLDSDGS | |
| | FFLYSKLTVSKEEWQQGFVFSCSVLHEALHSHYTQKSLSLSPG | |
| 108 | EVQLVESGGGLVQPGGSLRLSCAASGFTFTDFYMSWVRQAPGK | anti-TREM2/CH35.23.4 |
| | GLEWVSVIRNKANAYTAGYNPSVKGRFTISRDNSKNTLYLQMN | heavy chain with knob, |
| | SLRAEDTAVYYCARLTYGFDYWGQGTLVTVSSASTKGPSVFPL | LALAPG, N297G, and LS |
| | APSSKSTSGGTAALGCLVKDYFPEPVTVSWNSGALTSGVHTFPA | mutations, truncated |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYGSTYRVVS | |
| | VLTVLHQDWLNGKEYKCKVSNKALGAPIEKTISKAKGQPREPQ | |
| | VYTLPPSRDELTKNQVSLWCLVKGFYPSDIAVEWESYGTEWSN | |
| | YKTTPPVLDSDGSFFLYSKLTVSKEEWQQGFVFSCSVLHEALHS | |
| | HYTQKSLSLSPG | |
| 109 | EIVMTQSPATLSVSPGERATLSCRASQSVSSNLAWFQQKPGQAP | Clone A V_{L} |
| | RLLIYGASTRATGIPARFSGSGSGTEFTLTISSLQPEDFAVYYCLQ | |
| | DNNFPPTFGQGTKVDIK | |
| 110 | RASQSVSSNLA | Clone A CDR-L1 |
| 111 | GASTRAT | Clone A CDR-L2 |
| 112 | LQDNNFPPT | Clone A CDR-L3 |
| 113 | EVQLVQSGAEVKKPGESLKISCKGSGYSFTSYWIGWVRQMPGK | Clone A V_{H} |
| | GLEWMGIIYPGDADARYSPSFQGQVTISADKSISTAYLQWSSLK | |
| | ASDTAMYFCARRRQGIFGDALDFWGQGTLVTVSS | |
| 114 | SYWIG | Clone A CDR-H1 |
| 115 | IIYPGDADARYSPSFQG | Clone A CDR-H2 |
| 116 | RRQGIFGDALDF | Clone A CDR-H3 |
| 117 | DIQMTQSPSSVSASVGDRVTITCRASQGISNWLAWYQQKPGKAP | Clone B V_{L} |
| | KLLIYAASSLQVGVPLRFSGSGSGTDFTLTISSLQPEDFATYYCQ | |
| | QADSFPRNFGQGTKLEIK | |
| 118 | RASQGISNWLA | Clone B CDR-L1 |
| 119 | AASSLQV | Clone B CDR-L2 |
| 120 | QQADSFPRN | Clone B CDR-L3 |
| 121 | EVQLVQSGAEVKKPGESLKISCKGSGHSFTNYWIAWVRQMPGK | Clone B V_{H} |
| | GLEWMGIIYPGDSDTRYSPSFQGQVTISADKSISTAYLQWSSLKA | |
| | SDTAVYFCARQRTFYYDSSGYFDYWGQGTLVTVSS | |
| 122 | NYWIA | Clone B CDR-H1 |
| 123 | IIYPGDSDTRYSPSFQG | Clone B and Clone C CDR-H2 |
| 124 | QRTFYYDSSGYFDY | Clone B CDR-H3 |
| 125 | DIQMTQSPSSVSASVGDRVTITCRASQGISSWLAWYQQKPGKAP | Clone C V_{L} |
| | KLLIYAASSLQNGVPSRFSGSGSGTDFTLTISSLQPEDFATYFCQQ | |
| | ADSFPRTFGQGTKLEIK | |
| 126 | RASQGISSWLA | Clone C CDR-L1 |
| 127 | AASSLQN | Clone C CDR-L2 |
| 128 | QQADSFPRT | Clone C CDR-L3 |
| 129 | EVQLVQSGAEVKKPGESLKISCKGSGYSFTSYWIAWVRQMPGK | Clone C V_{H} |
| | GLEWMGIIYPGDSDTRYSPSFQGQVTISADKSISTAYLQWSSLKA | |
| | SDTAMYFCARQRTFYYDSSDYFDYWGQGTLVTVSS | |
| 130 | SYWIA | Clone C CDR-H1 |
| 131 | QRTFYYDSSDYFDY | Clone C CDR-H3 |
| 132 | DVVMTQSPDSLAVSLGERATINCRSSQSLVHSNRYTYLHWYQQ | Clone D V_{L} |
| | KPGQSPKLLIYKVSNRFSGVPDRFSGSGSGTDFTLKISRVEAEDV | |
| | GVYYCSQSTRVPYTFGQGTKLEIK | |
| 133 | RSSQSLVHSNRYTYLH | Clone D CDR-L1 |
| 134 | KVSNRFS | Clone D CDR-L2 |
| 135 | SQSTRVPYT | Clone D CDR-L3 |
| 136 | QVQLVQSGAEVKKPGASVKVSCKASGYAFSSQWMNWVRQAP | Clone D V_{H} |
| | GQRLEWIGRIYPGGGDTNYAGKFQGRVTITADTSASTAYMELSS | |
| | LRSEDTAVYYCARLLRNQPGESYAMDYWGQGTLVTVSS | |
| 137 | YAFSSQWMN | Clone D CDR-H1 |
| 138 | RIYPGGGDTNYAGKFQG | Clone D CDR-H2 |
| 139 | ARLLRNQPGESYAMDY | Clone D CDR-H3 |
| 140 | APELLGGPSVFLFPPKPKDTLMISRTPEVTCVVVDVSHEDPEVKF | Fc polypeptide with hole |
| | NWYVDGVEVHNAKTKPREEQYNSTYRVVSVLTVLHQDWLNG | |
| | KEYKCKVSNKALPAPIEKTISKAKGQPREPQVYTLPPSRDELTKN | |
| | QVSLSCAVKGFYPSDIAVEWESNGQPENNYKTTPPVLDSDGSFF | |
| | LVSKLTVDKSRWQQGNVFSCSVMHEALHNHYTQKSLSLSPGK | |
| 141 | DKTHTCPPCPAPELLGGPSVFLFPPKPKDTLMISRTPEVTCVVVD | Fc polypeptide with hole and |
| | VSHEDPEVKFNWYVDGVEVHNAKTKPREEQYNSTYRVVSVLT | portion of human IgG1 hinge |
| | VLHQDWLNGKEYKCKVSNKALPAPIEKTISKAKGQPREPQVYT | sequence |
| | LPPSRDELTKNQVSLSCAVKGFYPSDIAVEWESNGQPENNYKTT | |
| | PPVLDSDGSFFLVSKLTVDKSRWQQGNVFSCSVMHEALHNHYT | |
| | QKSLSLSPGK | |
| 142 | EVQLVESGGGLVQPGGSLRLSCAASGFTFTDFYMSWVRQAPGK | Anti-TREM2 heavy chain with |
| | GLEWVSVIRNKANAYTAGYNPSVKGRFTISRDNSKNTLYLQMN | hole |
| | SLRAEDTAVYYCARLTYGFDYWGQGTLVTVSSASTKGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPELLGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLSCAVKGFYPSDIAVEWESNGQPENNY | |
| | KTTPPVLDSDGSFFLVSKLTVDKSRWQQGNVFSCSVMHEALHN | |
| | HYTQKSLSLSPGK | |
| 143 | APELLGGPSVFLFPPKPKDTLMISRTPEVTCVVVDVSHEDPEVKF | Fc polypeptide with knob |
| | NWYVDGVEVHNAKTKPREEQYNSTYRVVSVLTVLHQDWLNG | |
| | KEYKCKVSNKALPAPIEKTISKAKGQPREPQVYTLPPSRDELTKN | |
| | QVSLWCLVKGFYPSDIAVEWESNGQPENNYKTTPPVLDSDGSFF | |
| | LYSKLTVDKSRWQQGNVFSCSVMHEALHNHYTQKSLSLSPGK | |
| 144 | DKTHTCPPCPAPELLGGPSVFLFPPKPKDTLMISRTPEVTCVVVD | Fc polypeptide with knob and |
| | VSHEDPEVKFNWYVDGVEVHNAKTKPREEQYNSTYRVVSVLT | portion of human IgG1 hinge |
| | VLHQDWLNGKEYKCKVSNKALPAPIEKTISKAKGQPREPQVYT | sequence |
| | LPPSRDELTKNQVSLWCLVKGFYPSDIAVEWESNGQPENNYKTT | |
| | PPVLDSDGSFFLYSKLTVDKSRWQQGNVFSCSVMHEALHNHYT | |
| | QKSLSLSPGK | |
| 145 | EVQLVESGGGLVQPGGSLRLSCAASGFTFTDFYMSWVRQAPGK | Anti-TREM2 heavy chain with |
| | GLEWVSVIRNKANAYTAGYNPSVKGRFTISRDNSKNTLYLQMN | knob |
| | SLRAEDTAVYYCARLTYGFDYWGQGTLVTVSSASTKGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPELLGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLWCLVKGFYPSDIAVEWESNGQPENN | |
| | YKTTPPVLDSDGSFFLYSKLTVDKSRWQQGNVFSCSVMHEALH | |
| | NHYTQKSLSLSPGK | |
| 146 | APELLGGPSVFLFPPKPKDTLMISRTPEVTCVVVDVSHEDPEVKF | Clone CH3C.35.23.3, knob |
| | NWYVDGVEVHNAKTKPREEQYNSTYRVVSVLTVLHQDWLNG | |
| | KEYKCKVSNKALPAPIEKTISKAKGQPREPQVYTLPPSRDELTKN | |
| | QVSLWCLVKGFYPSDIAVEWESYGTEWVNYKTTPPVLDSDGSF | |
| | FLYSKLTVTKEEWQQGFVFSCSVMHEALHNHYTQKSLSLSPGK | |
| 147 | APELLGGPSVFLFPPKPKDTLMISRTPEVTCVVVDVSHEDPEVKF | Clone CH3C.35.23.4, knob |
| | NWYVDGVEVHNAKTKPREEQYNSTYRVVSVLTVLHQDWLNG | |
| | KEYKCKVSNKALPAPIEKTISKAKGQPREPQVYTLPPSRDELTKN | |
| | QVSLWCLVKGFYPSDIAVEWESYGTEWSNYKTTPPVLDSDGSFF | |
| | LYSKLTVSKEEWQQGFVFSCSVMHEALHNHYTQKSLSLSPGK | |
| 148 | GGSGGGGSGGGGSGGGGS | Linker |
| 149 | GGGGSGGGGSGGGGSGGGGSGGGGSGGGGSGG | Linker |
| 150 | AIQLTQSPSSLSASVGDRVTITCRASQSISSYLAWYQQKPGKAPK | Anti-TfR Light Chain |
| | LLIYRASTLASGVPSRFSGSGSGTDFTLTISSLQPEDFATYYCQQN | |
| | YASSNVDNTFGGGTKVEIKRTVAAPSVFIFPPSDEQLKSGTASVV | |
| | CLLNNFYPREAKVQWKVDNALQSGNSQESVTEQDSKDSTYSLS | |
| | STLTLSKADYEKHKVYACEVTHQGLSSPVTKSFNRGEC | |
| 151 | AIQLTQSPSSLSASVGDRVTITCRASQSISSYLAWYQQKPGKAPK | Anti-TfR VL |
| | LLIYRASTLASGVPSRFSGSGSGTDFTLTISSLQPEDFATYYCQQN | |
| | YASSNVDNTFGGGTKVEIK | |
| 152 | RASQSISSYLA | Anti-TfR CDR-L1 |
| 153 | YRASTLAS | Anti-TfR CDR-L2 |
| 154 | QQNYASSNVDNT | Anti-TfR CDR-L3 |
| 155 | QSMQESGPGLVKPSQTLSLTCTVSGFSLSSYAMSWIRQHPGKGL | Anti-TfR Fd |
| | EWIGYIWSGGSTDYASWAKGRVTISKTSTTVSLKLSSVTAADTA | |
| | VYYCARRYGTSYPDYGDAQGFDPWGQGTLVTVSSASTKGPSVF | |
| | PLAPSSKSTSGGTAALGCLVKDYFPEPVTVSWNSGALTSGVHTF | |
| | PAVLQSSGLYSLSSVVTVPSSSLGTQTYICNVNHKPSNTKVDKK | |
| | VEPKSC | |
| 156 | QSMQESGPGLVKPSQTLSLTCTVSGFSLSSYAMSWIRQHPGKGL | Anti-TfR V_{H} |
| | EWIGYIWSGGSTDYASWAKGRVTISKTSTTVSLKLSSVTAADTA | |
| | VYYCARRYGTSYPDYGDAQGFDPWGQGTLVTVSS | |
| 157 | TVSGFSLSSYAMS | Anti-TfR CDR-H1 |
| 158 | YIWSGGSTD | Anti-TfR CDR-H2 |
| 159 | ARRYGTSYPDYGDAQGFDP | Anti-TfR CDR-H3 |
| 160 | APELLGGPSVFLFPPKPKDTLMISRTPEVTCVVVDVSHEDPEVKF | Wild-type human Fc sequence |
| | NWYVDGVEVHNAKTKPREEQYNSTYRVVSVLTVLHQDWLNG | truncated |
| | KEYKCKVSNKALPAPIEKTISKAKGQPREPQVYTLPPSRDELTKN | |
| | QVSLTCLVKGFYPSDIAVEWESNGQPENNYKTTPPVLDSDGSFF | |
| | LYSKLTVDKSRWQQGNVFSCSVMHEALHNHYTQKSLSLSPG | |
| 161 | APELLGGPSVFLFPPKPKDTLMISRTPEVTCVVVDVSHEDPEVKF | Clone CH3C.35.23.3, truncated |
| | NWYVDGVEVHNAKTKPREEQYNSTYRVVSVLTVLHQDWLNG | |
| | KEYKCKVSNKALPAPIEKTISKAKGQPREPQVYTLPPSRDELTKN | |
| | QVSLTCLVKGFYPSDIAVEWESYGTEWVNYKTTPPVLDSDGSFF | |
| | LYSKLTVTKEEWQQGFVFSCSVMHEALHNHYTQKSLSLSPG | |
| 162 | APELLGGPSVFLFPPKPKDTLMISRTPEVTCVVVDVSHEDPEVKF | Clone CH3C.35.23.4, truncated |
| | NWYVDGVEVHNAKTKPREEQYNSTYRVVSVLTVLHQDWLNG | |
| | KEYKCKVSNKALPAPIEKTISKAKGQPREPQVYTLPPSRDELTKN | |
| | QVSLTCLVKGFYPSDIAVEWESYGTEWSNYKTTPPVLDSDGSFF | |
| | LYSKLTVSKEEWQQGFVFSCSVMHEALHNHYTQKSLSLSPG | |
| 163 | APELLGGPSVFLFPPKPKDTLMISRTPEVTCVVVDVSHEDPEVKF | Fc polypeptide with hole, |
| | NWYVDGVEVHNAKTKPREEQYNSTYRVVSVLTVLHQDWLNG | truncated |
| | KEYKCKVSNKALPAPIEKTISKAKGQPREPQVYTLPPSRDELTKN | |
| | QVSLSCAVKGFYPSDIAVEWESNGQPENNYKTTPPVLDSDGSFF | |
| | LVSKLTVDKSRWQQGNVFSCSVMHEALHNHYTQKSLSLSPG | |
| 164 | DKTHTCPPCPAPELLGGPSVFLFPPKPKDTLMISRTPEVTCVVVD | Fc polypeptide with hole and |
| | VSHEDPEVKFNWYVDGVEVHNAKTKPREEQYNSTYRVVSVLT | portion of human IgG1 hinge |
| | VLHQDWLNGKEYKCKVSNKALPAPIEKTISKAKGQPREPQVYT | sequence, truncated |
| | LPPSRDELTKNQVSLSCAVKGFYPSDIAVEWESNGQPENNYKTT | |
| | PPVLDSDGSFFLVSKLTVDKSRWQQGNVFSCSVMHEALHNHYT | |
| | QKSLSLSPG | |
| 165 | EVQLVESGGGLVQPGGSLRLSCAASGFTFTDFYMSWVRQAPGK | Anti-TREM2 heavy chain with |
| | GLEWVSVIRNKANAYTAGYNPSVKGRFTISRDNSKNTLYLQMN | hole, truncated |
| | SLRAEDTAVYYCARLTYGFDYWGQGTLVTVSSASTKGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPELLGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLSCAVKGFYPSDIAVEWESNGQPENNY | |
| | KTTPPVLDSDGSFFLVSKLTVDKSRWQQGNVFSCSVMHEALHN | |
| | HYTQKSLSLSPG | |
| 166 | APELLGGPSVFLFPPKPKDTLMISRTPEVTCVVVDVSHEDPEVKF | Fc polypeptide with knob, |
| | NWYVDGVEVHNAKTKPREEQYNSTYRVVSVLTVLHQDWLNG | truncated |
| | KEYKCKVSNKALPAPIEKTISKAKGQPREPQVYTLPPSRDELTKN | |
| | QVSLWCLVKGFYPSDIAVEWESNGQPENNYKTTPPVLDSDGSFF | |
| | LYSKLTVDKSRWQQGNVFSCSVMHEALHNHYTQKSLSLSPG | |
| 167 | DKTHTCPPCPAPELLGGPSVFLFPPKPKDTLMISRTPEVTCVVVD | Fc polypeptide with knob and |
| | VSHEDPEVKFNWYVDGVEVHNAKTKPREEQYNSTYRVVSVLT | portion of human IgG1 hinge |
| | VLHQDWLNGKEYKCKVSNKALPAPIEKTISKAKGQPREPQVYT | sequence, truncated |
| | LPPSRDELTKNQVSLWCLVKGFYPSDIAVEWESNGQPENNYKTT | |
| | PPVLDSDGSFFLYSKLTVDKSRWQQGNVFSCSVMHEALHNHYT | |
| | QKSLSLSPG | |
| 168 | EVQLVESGGGLVQPGGSLRLSCAASGFTFTDFYMSWVRQAPGK | Anti-TREM2 heavy chain with |
| | GLEWVSVIRNKANAYTAGYNPSVKGRFTISRDNSKNTLYLQMN | knob, truncated |
| | SLRAEDTAVYYCARLTYGFDYWGQGTLVTVSSASTKGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPELLGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLWCLVKGFYPSDIAVEWESNGQPENN | |
| | YKTTPPVLDSDGSFFLYSKLTVDKSRWQQGNVFSCSVMHEALH | |
| | NHYTQKSLSLSPG | |
| 169 | APELLGGPSVFLFPPKPKDTLMISRTPEVTCVVVDVSHEDPEVKF | Clone CH3C.35.23.3, knob, |
| | NWYVDGVEVHNAKTKPREEQYNSTYRVVSVLTVLHQDWLNG | truncated |
| | KEYKCKVSNKALPAPIEKTISKAKGQPREPQVYTLPPSRDELTKN | |
| | QVSLWCLVKGFYPSDIAVEWESYGTEWVNYKTTPPVLDSDGSF | |
| | FLYSKLTVTKEEWQQGFVFSCSVMHEALHNHYTQKSLSLSPG | |
| 170 | APELLGGPSVFLFPPKPKDTLMISRTPEVTCVVVDVSHEDPEVKF | Clone CH3C.35.23.4, knob, |
| | NWYVDGVEVHNAKTKPREEQYNSTYRVVSVLTVLHQDWLNG | truncated |
| | KEYKCKVSNKALPAPIEKTISKAKGQPREPQVYTLPPSRDELTKN | |
| | QVSLWCLVKGFYPSDIAVEWESYGTEWSNYKTTPPVLDSDGSFF | |
| | LYSKLTVSKEEWQQGFVFSCSVMHEALHNHYTQKSLSLSPG | |
| 171 | EVQLVESGGGLVQPGGSLRLSCAASGFTFTDFYMSWVRQAPGK | Anti-TREM2 heavy chain with |
| | GLEWVSVIRNKANAYTAGYNPSVKGRFTISRDNSKNTLYLQMN | knob, linked through a |
| | SLRAEDTAVYYCARLTYGFDYWGQGTLVTVSSASTKGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPELLGGPSVFLFPPKPKDTLMISRTPEVTCV | |
| | VVDVSHEDPEVKFNWYVDGVEVHNAKTKPREEQYNSTYRVVS | |
| | VLTVLHQDWLNGKEYKCKVSNKALPAPIEKTISKAKGQPREPQ | |
| | VYTLPPSRDELTKNQVSLWCLVKGFYPSDIAVEWESNGQPENN | |
| | YKTTPPVLDSDGSFFLYSKLTVDKSRWQQGNVFSCSVMHEALH | |
| | NHYTQKSLSLSPGGGSGGGGSGGGGSGGGGSAIQLTQSPSSLSAS | |
| | VGDRVTITCRASQSISSYLAWYQQKPGKAPKLLIYRASTLASGVP | |
| | SRFSGSGSGTDFTLTISSLQPEDFATYYCQQNYASSNVDNTFGGG | |
| | TKVEIKRTVAAPSVFIFPPSDEQLKSGTASVVCLLNNFYPREAKV | polypeptide linker (italics) |
| | QWKVDNALQSGNSQESVTEQDSKDSTYSLSSTLTLSKADYEKH | to an anti-TfR scFab |
| | KVYACEVTHQGLSSPVTKSFNRGECGGGGSGGGGSGGGGSGGG | (underlined) |
| | GSGGGGSGGGGSGGQSMQESGPGLVKPSQTLSLTCTVSGFSLSS | |
| | YAMSWIRQHPGKGLEWIGYIWSGGSTDYASWAKGRVTISKTST | |
| | TVSLKLSSVTAADTAVYYCARRYGTSYPDYGDAQGFDPWGQG | |
| | TLVTVSSASTKGPSVFPLAPSSKSTSGGTAALGCLVKDYFPEPVT | |
| | VSWNSGALTSGVHTFPAVLQSSGLYSLSSVVTVPSSSLGTQTYIC | |
| | NVNHKPSNTKVDKKVEPKSC | |
| 172 | G₄S | Linker |
| 173 | (G₄S)_{z1} | Linker, wherein z1 is 1 to 10 |
| 174 | EVQLVESGGGLVQPGGSLRLSCAASGFTFTDFYMSWVRQAPGK | Anti-TREM2 heavy chain with |
| | GLEWVSVIRNKANAYTAGYNPSVKGRFTISRDNSKNTLYLQMN | knob, LALAPG, N297G, and |
| | SLRAEDTAVYYCARLTYGFDYWGQGTLVTVSSASTKGPSVFPL | LS mutations |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYGSTYRVVS | |
| | VLTVLHQDWLNGKEYKCKVSNKALGAPIEKTISKAKGQPREPQ | |
| | VYTLPPSRDELTKNQVSLWCLVKGFYPSDIAVEWESNGQPENN | |
| | YKTTPPVLDSDGSFFLYSKLTVDKSRWQQGNVFSCSVLHEALHS | |
| | HYTQKSLSLSPGK | |
| 175 | EVQLVESGGGLVQPGGSLRLSCAASGFTFTDFYMSWVRQAPGK | Anti-TREM2 heavy chain with |
| | GLEWVSVIRNKANAYTAGYNPSVKGRFTISRDNSKNTLYLQMN | knob, LALAPG, N297G, and |
| | SLRAEDTAVYYCARLTYGFDYWGQGTLVTVSSASTKGPSVFPL | LS mutations, truncated |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCDKTHTCPPCPAPEAAGGPSVFLFPPKPKDTLMISRTPEVTCV | |
| | VVDVSHEDPEVKFNWYVDGVEVHNAKTKPREEQYGSTYRVVS | |
| | VLTVLHQDWLNGKEYKCKVSNKALGAPIEKTISKAKGQPREPQ | |
| | VYTLPPSRDELTKNQVSLWCLVKGFYPSDIAVEWESNGQPENN | |
| | YKTTPPVLDSDGSFFLYSKLTVDKSRWQQGNVFSCSVLHEALHS | |
| | HYTQKSLSLSPG | |

All publications, patents, and patent documents are incorporated by reference herein, as though individually incorporated by reference. The present disclosure has been described with reference to various specific and preferred embodiments and techniques. However, it should be understood that many variations and modifications may be made while remaining within the spirit and scope of the invention.

What is claimed is:

1. A molecule comprising:
(a) a first binding region that specifically binds to TREM2; and
(b) a second binding region that specifically binds to a transferrin receptor (TfR);
wherein the molecule is monovalent for binding to TREM2.

2. The molecule of claim 1, wherein the first binding region and/or the second binding region comprises an antibody variable region sequence.

3. The molecule of claim 2, wherein the first binding region and/or the second binding region comprises or consists of a Fv region.

4. The molecule of claim 3, wherein the first binding region and/or the second binding region is a single chain Fv (scFV) or a disulfide-stabilized Fv (dsFv).

5. The molecule of claim 2, wherein the first binding region and/or the second binding region comprises or consists of a Fab fragment.

6. The molecule of claim 5, wherein the first binding region and/or the second binding region is a Fab fragment.

7. The molecule of claim 5, wherein the first binding region is a Fab fragment.

8. The molecule of claim 5, wherein the first binding region and/or the second binding region is a single chain Fab fragment (scFab).

9. The molecule of claim 8, wherein the second binding region is a scFab.

10. The molecule of any one of claims 1-9, which further comprises a first Fc polypeptide.

11. The molecule of claim 10, which further comprises a second Fc polypeptide, wherein the second Fc polypeptide forms an Fc dimer with the first Fc polypeptide.

12. The molecule of claim 11, wherein the first binding region is linked to the first or the second Fc polypeptide.

13. The molecule of claim 12, wherein the first binding region is linked to the N-terminus of the first or the second Fc polypeptide.

14. The molecule of any one of claims 11-13, wherein the second binding region is linked to the first or the second Fc polypeptide.

15. The molecule of claim 14, wherein the second binding region is linked to the N-terminus of the first or the second Fc polypeptide.

16. The molecule of claim 14, wherein the second binding region is linked to the C-terminus of the first or the second Fc polypeptide.

17. The molecule of any one of claims 11-13, wherein the second binding region is comprised within the first or the second Fc polypeptide.

18. The molecule of any one of claims 1-17, which further comprises a third binding region that specifically binds to a target protein.

19. The molecule of claim 18, wherein the third binding region comprises an antibody variable region sequence.

20. The molecule of claim 19, wherein the third binding region comprises or consists of a Fv region.

21. The molecule of claim 20, wherein the third binding region is a scFV or a dsFv.

22. The molecule of claim 19, wherein the third binding region comprises or consists of a Fab fragment.

23. The molecule of claim 22, wherein the third binding region is a Fab fragment.

24. The molecule of claim 22, wherein the third binding region is a scFab.

25. The molecule of any one of claims 18-24, wherein the third binding region is linked to the first or the second Fc polypeptide.

26. The molecule of claim 25, wherein the third binding region is linked to the N-terminus of the first or the second Fc polypeptide.

27. The molecule of any one of claims 1-17, which does not comprise a third binding region.

28. The molecule of claim 11, wherein the first binding region is linked to the N-terminus of the first Fc polypeptide, and wherein the second binding region is linked to the C-terminus of the first Fc polypeptide.

29. The molecule of claim 11, wherein the first binding region is linked to the N-terminus of the first Fc polypeptide, and wherein the second binding region is comprised within the first or the second Fc polypeptide.

30. The molecule of claim 28 or 29, further comprising a third binding region that specifically binds to a target protein, wherein the third binding region is linked to the N-terminus of the second Fc polypeptide.

31. The molecule of claim 28 or 29, which does not comprise a third binding region.

32. The molecule of any one of claims 1-31, which is monovalent for binding to TfR.

33. The molecule of any one of claims 1-32, which is capable of modulating TREM2 activity to a greater degree than a corresponding molecule that does not bind to a transferrin receptor.

34. The molecule of claim 33, which is capable of enhancing TREM2 activity to a greater degree than a corresponding molecule that does not bind to a transferrin receptor.

35. The molecule of claim 34, which is capable of enhancing TREM2 activity by at least about 2-fold more than a corresponding molecule that does not bind to a transferrin receptor.

36. The molecule of any one of claims 1-35, which is capable of modulating TREM2 activity (e.g., enhancing TREM2 activity) by at least the same degree as a corresponding molecule that is bivalent for TREM2 binding and that does not bind to a transferrin receptor.

37. The molecule of any one of claims 1-36, which has an increased avidity to TREM2 as compared to a corresponding molecule that does not bind to a transferrin receptor.

38. The molecule of any one of claims 1-37, which is capable of enhancing signaling of TREM2 on a cell surface as compared to a corresponding molecule that does not bind to a transferrin receptor.

39. The molecule of any one of claims 1-38, which is capable of enhancing Syk phosphorylation as compared to a corresponding molecule that does not bind to a transferrin receptor.

40. The molecule of any one of claims 1-39, which has enhanced internalization and/or endosomal localization as compared to a corresponding molecule that does not bind to a transferrin receptor.

41. The molecule of claim any one of claims 1-40, wherein the first binding region comprises:
i. a CDR-H1 sequence comprising the sequence of G-F-T-F-T-α₆-F-Y-M-S(SEQ ID NO:28), wherein α₆ is D or N;
ii. a CDR-H2 sequence comprising the sequence of V-I-R-N-β₅-β₆-N-β₈-Y-T-β₁₁-β₁₂-Y-N-P-S-V-K-G (SEQ ID NO:29), wherein β₅ is K or R; β₆ is A or P; β₈ is A or G; β₁₁ is A or T; and β₁₂ is G or D;
iii. a CDR-H3 sequence comprising the sequence of γ₁-R-L-γ₄-Y-G-F-D-Y (SEQ ID NO:30), wherein γ₁ is A or T; and γ₄ is T or S;
iv. a CDR-L1 sequence comprising the sequence of Q-S-S-K-S-L-L-H-S-δ₁₀-G-K-T-Y-L-N(SEQ ID NO:31), wherein δ₁₀ is T or N;
v. a CDR-L2 sequence comprising the sequence of WMSTRAS (SEQ ID NO:8); and
vi. a CDR-L3 sequence comprising the sequence of Q-Q-F-L-E-ϕ₆-P-F-T (SEQ ID NO:32), wherein ϕ₆ is Y or F.

42. The molecule of claim 41, wherein the CDR-H1 sequence is SEQ ID NO:4 or 12.

43. The molecule of claim 41 or 42, wherein the CDR-H2 sequence is SEQ ID NO:25, 5, or 13.

44. The molecule of any one of claims 41-43, wherein the CDR-H3 sequence is SEQ ID NO:17, 6, or 14.

45. The molecule of any one of claims 41-44, wherein the CDR-L1 sequence is SEQ ID NO:23 or 7.

46. The molecule of any one of claims 41-45, wherein the CDR-L3 sequence is SEQ ID NO:18 or 9.

47. The molecule of any one of claims 41-46, wherein the first binding region comprises:
(a) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO:17, a CDR-L1 comprising the amino acid sequence of SEQ ID NO:23, a CDR-L2 comprising the amino acid sequence of SEQ ID NO:8, and a CDR-L3 comprising the amino acid sequence of SEQ ID NO: 18; or
(b) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:18; or
(c) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:17, a CDR-L1 comprising the amino acid sequence of SEQ ID NO:23, a CDR-L2 comprising the amino acid sequence of SEQ ID NO:8, and a CDR-L3 comprising the amino acid sequence of SEQ ID NO:18; or
(d) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO:17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:18; or
(e) a CDR-H1 comprising the amino acid sequence of SEQ ID NO:4, a CDR-H2 comprising the amino acid sequence of SEQ ID NO:5, a CDR-H3 comprising the amino acid sequence of SEQ ID NO:6, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9; or
(f) a CDR-H1 comprising the amino acid sequence of SEQ ID NO:12, a CDR-H2 comprising the amino acid sequence of SEQ ID NO:13, a CDR-H3 comprising the amino acid sequence of SEQ ID NO:14, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9; or
(g) a CDR-H1 comprising the amino acid sequence of SEQ ID NO:4, a CDR-H2 comprising the amino acid sequence of SEQ ID NO:25, a CDR-H3 comprising the amino acid sequence of SEQ ID NO:17, a CDR-L1 comprising the amino acid sequence of SEQ ID NO:7, a CDR-L2 comprising the amino acid sequence of SEQ ID NO:8, and a CDR-L3 comprising the amino acid sequence of SEQ ID NO:9.

48. The molecule of any one of claims 41-47, wherein the first binding region comprises a V_{H} sequence comprising a sequence that has at least 85% sequence identity to any one of SEQ ID NOS:24, 2, 10, 15, 19, 21, and 26.

49. The molecule of claim 48, wherein the V_{H} sequence comprises a sequence that has at least 90% or 95% sequence identity to SEQ ID NO:24.

50. The molecule of claim 49, wherein the V_{H} sequence comprises SEQ ID NO:24.

51. The molecule of claim 48, wherein the V_{H} sequence comprises a sequence that has at least 90% or 95% sequence identity to SEQ ID NO:15.

52. The molecule of claim 51, wherein the V_{H} sequence comprises SEQ ID NO:15.

53. The molecule of any one of claims 41-52, wherein the first binding region comprises a V_{L} sequence comprising a sequence that has at least 85% sequence identity to any one of SEQ ID NOS:22, 3, 11, 16, 20, and 27.

54. The molecule of claim 53, wherein the V_{L} sequence comprises a sequence that has at least 90% or 95% sequence identity to SEQ ID NO:22.

55. The molecule of claim 54, wherein the V_{L} sequence comprises SEQ ID NO:22.

56. The molecule of claim 53, wherein the V_{L} sequence comprises a sequence that has at least 90% or 95% sequence identity to SEQ ID NO:16.

57. The molecule of claim 56, wherein the V_{L} sequence comprises SEQ ID NO:16.

58. The molecule of claim 53, wherein the V_{L} sequence comprises a sequence that has at least 90% or 95% sequence identity to SEQ ID NO:27.

59. The molecule of claim 58, wherein the V_{L} sequence comprises SEQ ID NO:27.

60. The molecule of any one of claims 41-59, wherein the first binding region comprises:
(a) a V_{H} sequence comprising SEQ ID NO:24 and a V_{L} sequence comprising SEQ ID NO:22; or
(b) a V_{H} sequence comprising SEQ ID NO:15 and a V_{L} sequence comprising SEQ ID NO:16; or
(c) a V_{H} sequence comprising SEQ ID NO:19 and a V_{L} sequence comprising SEQ ID NO:20; or
(d) a V_{H} sequence comprising SEQ ID NO:21 and a V_{L} sequence comprising SEQ ID NO:20; or
(e) a V_{H} sequence comprising SEQ ID NO:19 and a V_{L} sequence comprising SEQ ID NO:22; or
(f) a V_{H} sequence comprising SEQ ID NO:21 and a V_{L} sequence comprising SEQ ID NO:22; or
(g) a V_{H} sequence comprising SEQ ID NO:24 and a V_{L} sequence comprising SEQ ID NO:20; or
(h) a V_{H} sequence comprising SEQ ID NO:26 and a V_{L} sequence comprising SEQ ID NO:20; or
(i) a V_{H} sequence comprising SEQ ID NO:26 and a V_{L} sequence comprising SEQ ID NO:22; or
(j) a V_{H} sequence comprising SEQ ID NO:2 and a V_{L} sequence comprising SEQ ID NO:3; or
(k) a V_{H} sequence comprising SEQ ID NO:10 and a V_{L} sequence comprising SEQ ID NO:11; or
(1) a V_{H} sequence comprising SEQ ID NO:24 and a V_{L} sequence comprising SEQ ID NO:27.

61. The molecule of any one of claims 1-40, wherein the first binding region comprises:
i. a CDR-H1 sequence comprising the sequence of SYWIG (SEQ ID NO: 114);
ii. a CDR-H2 sequence comprising the sequence of IIYPGDADARYSPSFQG (SEQ ID NO:115);
iii. a CDR-H3 sequence comprising the sequence of RRQGIFGDALDF (SEQ ID NO:116);
iv. a CDR-L1 sequence comprising the sequence of RASQSVSSNLA (SEQ ID NO:110);
v. a CDR-L2 sequence comprising the sequence of GASTRAT (SEQ ID NO:111); and
vi. a CDR-L3 sequence comprising the sequence of LQDNNFPPT (SEQ ID NO:112).

62. The molecule of claim 61, wherein the first binding region comprises a V_{H} sequence comprising a sequence that has at least 85%, 90%, or 95% sequence identity to SEQ ID NO:113.

63. The molecule of claim 62, wherein the V_{H} sequence comprises SEQ ID NO:113.

64. The molecule of any one of claims 61-63, wherein the first binding region comprises a V_{L} sequence comprising a sequence that has at least 85%, 90%, or 95% sequence identity to SEQ ID NO:109.

65. The molecule of claim 64, wherein the V_{L} sequence comprises SEQ ID NO:109.

66. The molecule of any one of claims 61-65, wherein the first binding region comprises a V_{H} sequence comprising SEQ ID NO:113 and a V_{L} sequence comprising SEQ ID NO:109.

67. The molecule of any one of claims 1-40, wherein the first binding region comprises:
i. a CDR-H1 sequence comprising the sequence of NYWIA (SEQ ID NO: 122);
ii. a CDR-H2 sequence comprising the sequence of IIYPGDSDTRYSPSFQG (SEQ ID NO:123);
iii. a CDR-H3 sequence comprising the sequence of QRTFYYDSSGYFDY (SEQ ID NO:124);
iv. a CDR-L1 sequence comprising the sequence of RASQGISNWLA (SEQ ID NO:118);
v. a CDR-L2 sequence comprising the sequence of AASSLQV (SEQ ID NO:119); and
vi. a CDR-L3 sequence comprising the sequence of QQADSFPRN (SEQ ID NO:120).

68. The molecule of claim 67, wherein the first binding region comprises a V_{H} sequence comprising a sequence that has at least 85%, 90%, or 95% sequence identity to SEQ ID NO:121.

69. The molecule of claim 68, wherein the V_{H} sequence comprises SEQ ID NO:121.

70. The molecule of any one of claims 67-69, wherein the first binding region comprises a V_{L} sequence comprising a sequence that has at least 85%, 90%, or 95% sequence identity to SEQ ID NO: 117.

71. The molecule of claim 70, wherein the V_{L} sequence comprises SEQ ID NO:117.

72. The molecule of any one of claims 67-71, wherein the first binding region comprises a V_{H} sequence comprising SEQ ID NO:121 and a V_{L} sequence comprising SEQ ID NO:117.

73. The molecule of claim of any one of claims 1-40, wherein the first binding region comprises:
i. a CDR-H1 sequence comprising the sequence of SYWIA (SEQ ID NO:130);
ii. a CDR-H2 sequence comprising the sequence of IIYPGDSDTRYSPSFQG (SEQ ID NO: 123);
iii. a CDR-H3 sequence comprising the sequence of QRTFYYDSSDYFDY (SEQ ID NO:131);
iv. a CDR-L1 sequence comprising the sequence of RASQGISSWLA (SEQ ID NO:126);
v. a CDR-L2 sequence comprising the sequence of AASSLQN (SEQ ID NO:127); and
vi. a CDR-L3 sequence comprising the sequence of QQADSFPRT (SEQ ID NO:128).

74. The molecule of claim 73, wherein the first binding region comprises a V_{H} sequence comprising a sequence that has at least 85%, 90%, or 95% sequence identity to SEQ ID NO: 129.

75. The molecule of claim 74, wherein the V_{H} sequence comprises SEQ ID NO:129.

76. The molecule of any one of claims 73-75, wherein the first binding region comprises a V_{L} sequence comprising a sequence that has at least 85%, 90%, or 95% sequence identity to SEQ ID NO:125.

77. The molecule of claim 76, wherein the V_{L} sequence comprises SEQ ID NO:125.

78. The molecule of any one of claims 73-77, wherein the first binding region comprises a V_{H} sequence comprising SEQ ID NO:129 and a V_{L} sequence comprising SEQ ID NO:125.

79. The molecule of any one of claims 1-40, wherein the first binding region comprises:
i. a CDR-H1 sequence comprising the sequence of YAFSSQWMN (SEQ ID NO:137);
ii. a CDR-H2 sequence comprising the sequence of RIYPGGGDTNYAGKFQG (SEQ ID NO:138);
iii. a CDR-H3 sequence comprising the sequence of ARLLRNQPGESYAMDY (SEQ ID NO:139);
iv. a CDR-L1 sequence comprising the sequence of RSSQSLVHSNRYTYLH (SEQ ID NO:133);
v. a CDR-L2 sequence comprising the sequence of KVSNRFS (SEQ ID NO:134); and
vi. a CDR-L3 sequence comprising the sequence of SQSTRVPYT (SEQ ID NO: 135).

80. The molecule of claim 79, wherein the first binding region comprises a V_{H} sequence comprising a sequence that has at least 85%, 90%, or 95% sequence identity to SEQ ID NO:136.

81. The molecule of claim 80, wherein the V_{H} sequence comprises SEQ ID NO:136.

82. The molecule of any one of claims 79-81, wherein the first binding region comprises a V_{L} sequence comprising a sequence that has at least 85%, 90%, or 95% sequence identity to SEQ ID NO:132.

83. The molecule of claim 82, wherein the V_{L} sequence comprises SEQ ID NO:132.

84. The molecule of any one of claims 79-83, wherein the first binding region comprises a V_{H} sequence comprising SEQ ID NO:136 and a V_{L} sequence comprising SEQ ID NO:132.

85. The molecule of any one of claims 11-84, wherein the first Fc polypeptide and the second Fc polypeptide each contain modifications that promote heterodimerization, and optionally, wherein the first Fc polypeptide and/or the second Fc polypeptide further comprise one or more modifications that reduce effector function and/or extend serum half-life.

86. The molecule of claim 85, wherein the first Fc polypeptide has a T366W substitution and the second Fc polypeptide has T366S, L368A, and Y407V substitutions, according to EU numbering.

87. The molecule of claim 85, wherein the first Fc polypeptide has T366S, L368A, and Y407V substitutions and the second Fc polypeptide has a T366W substitution, according to EU numbering.

88. The molecule of any one of claims 11-87, wherein the first Fc polypeptide and/or the second Fc polypeptide comprise one or more substitutions independently selected from the group consisting of L234A, L235A, R292C, N297G, V302C, P329G, P331S, D356E, L358M, M428L, E430G, and N434S, according to EU numbering.

89. The molecule of claim 88, wherein the first Fc polypeptide and/or the second Fc polypeptide comprise L234A and L235A substitutions, according to EU numbering; or comprise L234A, L235A and P329G substitutions, according to EU numbering; or comprise L234A, L235A, N297G, and P329G substitutions, according to EU numbering.

90. The molecule of claim 88 or 89, wherein the first Fc polypeptide and/or the second Fc polypeptide comprise M428L and N434S substitutions, according to EU numbering.

91. The molecule of any one of claims 17-27 and 29-90, wherein the second binding region is comprised within the first or the second Fc polypeptide, and wherein the first Fc polypeptide or the second polypeptide comprises: Trp, Leu, or Glu at position 380; Tyr or Phe at position 384; Thr at position 386; Glu at position 387; Trp at position 388; Ser, Ala, or Val at position 389; Ser or Asn at position 390; Thr or Ser at position 413; Glu or Ser at position 415; Glu at position 416; and Phe at position 421, according to EU numbering.

92. The molecule of claim 91, wherein the first Fc polypeptide comprises: Trp, Leu, or Glu at position 380; Tyr or Phe at position 384; Thr at position 386; Glu at position 387; Trp at position 388; Ser, Ala, or Val at position 389; Ser or Asn at position 390; Thr or Ser at position 413; Glu or Ser at position 415; Glu at position 416; and Phe at position 421, according to EU numbering.

93. The molecule of claim 92, wherein the first Fc polypeptide 1) comprises a sequence that has at least 80%, 85%, 90%, or 95% sequence identity to a sequence selected from the group consisting of SEQ ID NOS:47, 68, 38, 39, 46, 49, 50, 61, 63, 70, 84, 87, 93, 95, 99, 105, 107, 140, 143, 146, 147, 160-163, 166, 169, and 170; and 2) comprises: Trp, Leu, or Glu at position 380; Tyr or Phe at position 384; Thr at position 386; Glu at position 387; Trp at position 388; Ser, Ala, or Val at position 389; Ser or Asn at position 390; Thr or Ser at position 413; Glu or Ser at position 415; Glu at position 416; and Phe at position 421, according to EU numbering.

94. The molecule of claim 92, wherein the first Fc polypeptide 1) comprises a sequence that has at least 90% or 95% sequence identity to a sequence selected from the group consisting of SEQ ID NOS: 47, 68, 46, 49, 50, 70, 93, 95, 105, 107, 146, 147, 161, 162, 169, and 170; and 2) comprises: Trp, Leu, or Glu at position 380; Tyr or Phe at position 384; Thr at position 386; Glu at position 387; Trp at position 388; Ser, Ala, or Val at position 389; Ser or Asn at position 390; Thr or Ser at position 413; Glu or Ser at position 415; Glu at position 416; and Phe at position 421, according to EU numbering.

95. The molecule of claim 92, wherein the first Fc polypeptide 1) comprises a sequence that has at least 90% or 95% sequence identity to a sequence selected from the group consisting of SEQ ID NOS: 47, 68, 50, 70, 93, 95, 105, 107, 146, 147, 169, and 170; and 2) comprises: Trp, Leu, or Glu at position 380; Tyr or Phe at position 384; Thr at position 386; Glu at position 387; Trp at position 388; Ser, Ala, or Val at position 389; Ser or Asn at position 390; Thr or Ser at position 413; Glu or Ser at position 415; Glu at position 416; and Phe at position 421, according to EU numbering.

96. The molecule of any one of claims 92-95, wherein the second Fc polypeptide comprises a sequence that has at least 90% or 95% sequence identity to a sequence selected from the group consisting of SEQ ID NOS: 39, 63, 38, 61, 84, 87, 99, 140, 143, 160, 163, and 166.

97. The molecule of any one of claims 92-95, wherein the second Fc polypeptide comprises a sequence that has at least 90% or 95% sequence identity to a sequence selected from the group consisting of SEQ ID NOS: 39, 63, 61, 84, 87, 99, 140, and 163.

98. The molecule of any one of claims 95-97, wherein the first Fc polypeptide comprises a sequence selected from the group consisting of SEQ ID NOS:47, 68, 93, and 105, and the second Fc polypeptide comprises a sequence selected from the group consisting of SEQ ID NOS:39, 63, 87, 99, 61 and 84.

99. The molecule of claim 98, comprising:
(i) a heavy chain (HC) comprising 1) a V_{H} comprising a sequence selected from the group consisting of SEQ ID NOS:24, 113, 121, 129, and 136; and 2) the first Fc polypeptide comprising SEQ ID NO:47;
(ii) the second Fc polypeptide comprising SEQ ID NO:39 or 61; and
(iii) a light chain comprising a V_{L} comprising a sequence selected from the group consisting of SEQ ID NOS:22, 109, 117, 125, and 132.

100. The molecule of claim 98 comprising:
(i) a heavy chain (HC) comprising 1) a V_{H} comprising a sequence selected from the group consisting of SEQ ID NOS:24, 113, 121, 129, and 136; and 2) the first Fc polypeptide comprising SEQ ID NO:68;
(ii) the second Fc polypeptide comprising SEQ ID NO:63 or 84; and
(iii) a light chain comprising a V_{L} comprising a sequence selected from the group consisting of SEQ ID NOS:22, 109, 117, 125, and 132.

101. The molecule of claim 98, comprising:
(i) a heavy chain (HC) comprising 1) a V_{H} comprising a sequence selected from the group consisting of SEQ ID NOS:24, 113, 121, 129, and 136; and 2) the first Fc polypeptide comprising SEQ ID NO:93;
(ii) the second Fc polypeptide comprising SEQ ID NO:87; and
(iii) a light chain comprising a V_{L} comprising a sequence selected from the group consisting of SEQ ID NOS:22, 109, 117, 125, and 132.

102. The molecule of claim 98, comprising:
(i) a heavy chain (HC) comprising 1) a V_{H} comprising a sequence selected from the group consisting of SEQ ID NOS:24, 113, 121, 129, and 136; and 2) the first Fc polypeptide comprising SEQ ID NO:105;
(ii) the second Fc polypeptide comprising SEQ ID NO:99; and
(iii) a light chain comprising a V_{L} comprising a sequence selected from the group consisting of SEQ ID NOS:22, 109, 117, 125, and 132.

103. The molecule of any one of claims 99-102, wherein:
(a) the V_{H} comprises SEQ ID NO:24 and the V_{L} comprises SEQ ID NO:22;
(b) the V_{H} comprises SEQ ID NO:113 and the V_{L} comprises SEQ ID NO: 109;
(c) the V_{H} comprises SEQ ID NO:121 and the V_{L} comprises SEQ ID NO:117;
(d) the V_{H} comprises SEQ ID NO:129 and the V_{L} comprises SEQ ID NO:125; or
(e) the V_{H} comprises SEQ ID NO:136 and the V_{L} comprises SEQ ID NO:132.

104. The molecule of claim 103, comprising:
(i) a heavy chain (HC) that comprises or consists of the amino acid sequence set forth in SEQ ID NO:48;
(ii) the second Fc polypeptide that comprises or consists of the amino acid sequence set forth in SEQ ID NO:86 or 85; and
(iii) a light chain (LC) that comprises or consists of the amino acid sequence set forth in SEQ ID NO:54.

105. The molecule of claim 103, comprising:
(i) a heavy chain (HC) that comprises or consists of the amino acid sequence set forth in SEQ ID NO:69;
(ii) the second Fc polypeptide that comprises or consists of the amino acid sequence set forth in SEQ ID NO:98 or 97; and
(iii) a light chain (LC) that comprises or consists of the amino acid sequence set forth in SEQ ID NO:54.

106. The molecule of claim 103, comprising:
(i) a heavy chain (HC) that comprises or consists of the amino acid sequence set forth in SEQ ID NO:94;
(ii) the second Fc polypeptide that comprises or consists of the amino acid sequence set forth in SEQ ID NO:88; and
(iii) a light chain (LC) that comprises or consists of the amino acid sequence set forth in SEQ ID NO:54.

107. The molecule of claim 103, comprising:
(i) a heavy chain (HC) that comprises or consists of the amino acid sequence set forth in SEQ ID NO:106;
(ii) the second Fc polypeptide that comprises or consists of the amino acid sequence set forth in SEQ ID NO: 100; and
(iii) a light chain (LC) that comprises or consists of the amino acid sequence set forth in SEQ ID NO:54.

108. The molecule of any one of claims 95-97, wherein the first Fc polypeptide comprises a sequence selected from the group consisting of SEQ ID NOS:50, 95, 70, and 107, and the second Fc polypeptide comprises a sequence selected from the group consisting of SEQ ID NO:39, 87, 63, and 99.

109. The molecule of claim 108, comprising:
(i) a heavy chain (HC) comprising 1) a V_{H} comprising a sequence selected from the group consisting of SEQ ID NOS:24, 113, 121, 129, and 136; and 2) the first Fc polypeptide comprising SEQ ID NO:50;
(ii) the second Fc polypeptide comprising SEQ ID NO:39; and
(iii) a light chain comprising a V_{L} comprising a sequence selected from the group consisting of SEQ ID NOS:22, 109, 117, 125, and 132.

110. The molecule of claim 108, comprising:
(i) a heavy chain (HC) comprising 1) a V_{H} comprising a sequence selected from the group consisting of SEQ ID NOS:24, 113, 121, 129, and 136; and 2) the first Fc polypeptide comprising SEQ ID NO:70;
(ii) the second Fc polypeptide comprising SEQ ID NO:63; and
(iii) a light chain comprising a V_{L} comprising a sequence selected from the group consisting of SEQ ID NOS:22, 109, 117, 125, and 132.

111. The molecule of claim 108, comprising:
(i) a heavy chain (HC) comprising 1) a V_{H} comprising a sequence selected from the group consisting of SEQ ID NOS:24, 113, 121, 129, and 136; and 2) the first Fc polypeptide comprising SEQ ID NO:95;
(ii) the second Fc polypeptide comprising SEQ ID NO:87; and
(iii) a light chain comprising a V_{L} comprising a sequence selected from the group consisting of SEQ ID NOS:22, 109, 117, 125, and 132.

112. The molecule of claim 108, comprising:
(i) a heavy chain (HC) comprising 1) a V_{H} comprising a sequence selected from the group consisting of SEQ ID NOS:24, 113, 121, 129, and 136; and 2) the first Fc polypeptide comprising SEQ ID NO:107;
(ii) the second Fc polypeptide comprising SEQ ID NO:99; and
(iii) a light chain comprising a V_{L} comprising a sequence selected from the group consisting of SEQ ID NOS:22, 109, 117, 125, and 132.

113. The molecule of any one of claims 109-112, wherein:
(a) the V_{H} comprises SEQ ID NO:24 and the V_{L} comprises SEQ ID NO:22;
(b) the V_{H} comprises SEQ ID NO:113 and the V_{L} comprises SEQ ID NO:109;
(c) the V_{H} comprises SEQ ID NO:121 and the V_{L} comprises SEQ ID NO:117;
(d) the V_{H} comprises SEQ ID NO:129 and the V_{L} comprises SEQ ID NO: 125; or
(e) the V_{H} comprises SEQ ID NO:136 and the V_{L} comprises SEQ ID NO:132.

114. The molecule of claim 113, comprising:
(i) a heavy chain (HC) that comprises or consists of the amino acid sequence set forth in SEQ ID NO:51;
(ii) the second Fc polypeptide that comprises or consists of the amino acid sequence set forth in SEQ ID NO:86; and
(iii) a light chain (LC) that comprises or consists of the amino acid sequence set forth in SEQ ID NO:54.

115. The molecule of claim 113, comprising:
(i) a heavy chain (HC) that comprises or consists of the amino acid sequence set forth in SEQ ID NO:71;
(ii) the second Fc polypeptide that comprises or consists of the amino acid sequence set forth in SEQ ID NO:98; and
(iii) a light chain (LC) that comprises or consists of the amino acid sequence set forth in SEQ ID NO:54.

116. The molecule of claim 113, comprising:
(i) a heavy chain (HC) that comprises or consists of the amino acid sequence set forth in SEQ ID NO:96;
(ii) the second Fc polypeptide that comprises or consists of the amino acid sequence set forth in SEQ ID NO:88; and
(iii) a light chain (LC) that comprises or consists of the amino acid sequence set forth in SEQ ID NO:54.

117. The molecule of claim 113, comprising:
(i) a heavy chain (HC) that comprises or consists of the amino acid sequence set forth in SEQ ID NO:108;
(ii) the second Fc polypeptide that comprises or consists of the amino acid sequence set forth in SEQ ID NO: 100; and
(iii) a light chain (LC) that comprises or consists of the amino acid sequence set forth in SEQ ID NO:54.

118. The molecule of any one of claims 14-16, 18-28 and 30-90, wherein the second binding region is linked to the first or the second Fc polypeptide, and wherein the second binding region comprises:
i. a CDR-H1 sequence comprising the sequence of TVSGFSLSSYAMS (SEQ ID NO:157);
ii. a CDR-H2 sequence comprising the sequence of YIWSGGSTD (SEQ ID NO:158);
iii. a CDR-H3 sequence comprising the sequence of ARRYGTSYPDYGDAQGFDP (SEQ ID NO:159);
iv. a CDR-L1 sequence comprising the sequence of RASQSISSYLA (SEQ ID NO:152);
v. a CDR-L2 sequence comprising the sequence YRASTLAS (SEQ ID NO:153); and
vi. a CDR-L3 sequence comprising the sequence of QQNYASSNVDNT (SEQ ID NO:154).

119. The molecule of claim 118, wherein the second binding region comprises a V_{H} sequence comprising a sequence that has at least 85%, 90%, or 95% sequence identity to SEQ ID NO: 156.

120. The molecule of claim 119, wherein the V_{H} sequence comprises SEQ ID NO:156.

121. The molecule of any one of claims 118-120, wherein the second binding region comprises a V_{L} sequence comprising a sequence that has at least 85%, 90%, or 95% sequence identity to SEQ ID NO:151.

122. The molecule of claim 121, wherein the V_{L} sequence comprises SEQ ID NO:151.

123. The molecule of any one of claims 118-122, wherein the second binding region comprises a V_{H} sequence comprising SEQ ID NO:156 and a V_{L} sequence comprising SEQ ID NO:151.

124. The molecule of any one of claims 118-123, wherein the second binding region is linked to the C-terminus of the first or the second Fc polypeptide.

125. The molecule of any one of claims 118-123, wherein the second binding region is linked to the C-terminus of the first Fc polypeptide.

126. The molecule of any one of claims 118-125, wherein the first and/or second Fc polypeptide comprises a sequence that has at least 90% or 95% sequence identity to a sequence selected from the group consisting of SEQ ID NOS: 38, 140, 143, 160, 163, and 166.

127. The molecule of claim 126, wherein the first Fc polypeptide comprises a sequence that has at least 90% or 95% sequence identity to a sequence selected from the group consisting of SEQ ID NOS: 143 and 166.

128. The molecule of claim 127, wherein the second Fc polypeptide comprises a sequence that has at least 90% or 95% sequence identity to a sequence selected from the group consisting of SEQ ID NOS: 140 and 163.

129. The molecule of any one of claims 118-128, wherein the first Fc polypeptide comprises SEQ ID NO: 143 or 166, and the second Fc polypeptide comprises SEQ ID NO:140 or 163.

130. The molecule of claim 129, comprising:
(i) a heavy chain (HC) linked to the second binding region (SBR) comprising 1) a V_{H} comprising a sequence selected from the group consisting of SEQ ID NOS:24, 113, 121, 129, and 136; 2) the first Fc polypeptide comprising SEQ ID NO:166; and 3) the SBR comprising a SBR V_{H} sequence comprising SEQ ID NO:156 and a SBR V_{L} sequence comprising SEQ ID NO:151;
(ii) the second Fc polypeptide comprising SEQ ID NO:140; and
(iii) a light chain comprising a V_{L} comprising a sequence selected from the group consisting of SEQ ID NOS:22, 109, 117, 125, and 132.

131. The molecule of claim 129, comprising:
(i) a heavy chain (HC) linked to the second binding region (SBR) comprising 1) a V_{H} comprising a sequence selected from the group consisting of SEQ ID NOS:24, 113, 121, 129, and 136; 2) the first Fc polypeptide comprising SEQ ID NO:166; and 3) the SBR comprising a SBR V_{H} sequence comprising SEQ ID NO:156 and a SBR V_{L} sequence comprising SEQ ID NO:151;
(ii) the second Fc polypeptide comprising SEQ ID NO:163; and
(iii) a light chain comprising a V_{L} comprising a sequence selected from the group consisting of SEQ ID NOS:22, 109, 117, 125, and 132.

132. The molecule of any one of claims 130-131, wherein:
(a) the V_{H} comprises SEQ ID NO:24 and the V_{L} comprises SEQ ID NO:22;
(b) the V_{H} comprises SEQ ID NO:113 and the V_{L} comprises SEQ ID NO: 109;
(c) the V_{H} comprises SEQ ID NO:121 and the V_{L} comprises SEQ ID NO:117;
(d) the V_{H} comprises SEQ ID NO:129 and the V_{L} comprises SEQ ID NO: 125; or
(e) the V_{H} comprises SEQ ID NO:136 and the V_{L} comprises SEQ ID NO:132.

133. The molecule of claim 132, comprising:
(i) the heavy chain (HC) linked to the second binding region (SBR), which comprises from N-terminus to C-terminus: 1) the heavy chain comprising or consisting of the amino acid sequence set forth in SEQ ID NO:168; 2) a first polypeptide linker; and 3) the SBR, which comprises from N-terminus to C-terminus: a) a SBR light chain comprising or consisting of the amino acid sequence set forth in SEQ ID NO: 150; b) a second polypeptide linker; and c) a Fd region comprising or consisting of the amino acid sequence set forth in SEQ ID NO: 155;
(ii) the second Fc polypeptide that comprises or consists of the amino acid sequence set forth in SEQ ID NO:141; and
(iii) the light chain (LC) that comprises or consists of the amino acid sequence set forth in SEQ ID NO:54.

134. The molecule of claim 132, comprising:
(i) the heavy chain (HC) linked to the second binding region (SBR), which comprises from N-terminus to C-terminus: 1) the heavy chain comprising or consisting of the amino acid sequence set forth in SEQ ID NO:168; 2) a first polypeptide linker; and 3) the SBR, which comprises from N-terminus to C-terminus: a) a SBR light chain comprising or consisting of the amino acid sequence set forth in SEQ ID NO: 150; b) a second polypeptide linker; and c) a Fd region comprising or consisting of the amino acid sequence set forth in SEQ ID NO: 155;
(ii) the second Fc polypeptide that comprises or consists of the amino acid sequence set forth in SEQ ID NO: 164; and
(iii) the light chain (LC) that comprises or consists of the amino acid sequence set forth in SEQ ID NO:54.

135. The molecule of claim 132, comprising:
(i) the heavy chain (HC) linked to the second binding region that comprises or consists of SEQ ID NO:171;
(ii) the second Fc polypeptide that comprises or consists of the amino acid sequence set forth in SEQ ID NO:141; and
(iii) the light chain (LC) that comprises or consists of the amino acid sequence set forth in SEQ ID NO:54.

136. The molecule of claim 132, comprising:
(i) the heavy chain (HC) linked to the second binding region that comprises or consists of SEQ ID NO:171;
(ii) the second Fc polypeptide that comprises or consists of the amino acid sequence set forth in SEQ ID NO: 164; and
(iii) the light chain (LC) that comprises or consists of the amino acid sequence set forth in SEQ ID NO:54.

137. A pharmaceutical composition comprising the molecule of any one of claims 1-136 and a pharmaceutically acceptable carrier.

138. A method of treating a neurodegenerative disease in a subject, comprising administering to the subject the molecule of any one of claims 1-136 or the pharmaceutical composition of claim 137.

139. The method of claim 138, wherein the neurodegenerative disease is selected from the group consisting of: Alzheimer's disease, primary age-related tauopathy, progressive supranuclear palsy (PSP), frontotemporal dementia, frontotemporal dementia with parkinsonism linked to chromosome 17, argyrophilic grain dementia, amyotrophic lateral sclerosis, amyotrophic lateral sclerosis/parkinsonism-dementia complex of Guam (ALS-PDC), corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, hereditary diffuse leukoencephalopathy with spheroids (HDLS), Huntington's disease, inclusion-body myositis, multiple system atrophy, myotonic dystrophy, Nasu-Hakola disease, neurofibrillary tangle-predominant dementia, Niemann-Pick disease type C, pallido-ponto-nigral degeneration, Parkinson's disease, Pick's disease, postencephalitic parkinsonism, prion protein cerebral amyloid angiopathy, progressive subcortical gliosis, subacute sclerosing panencephalitis, adult-onset leukoencephalopathy with axonal spheroids and pigmented glia (ALSP), and tangle only dementia.

140. A method of enhancing TREM2 activity in a subject having a neurodegenerative disease, comprising administering to the subject the molecule of any one of claims 1-136 or the pharmaceutical composition of claim 137.

141. An isolated polynucleotide comprising a nucleotide sequence encoding the molecule of any one of claims 1-136.

142. An isolated polynucleotide comprising a nucleotide sequence encoding a light chain, a heavy chain, and/or a Fc polypeptide of any one of claims 99-107, 109-117 and 130-136.

143. A plurality of polynucleotides, wherein the plurality comprises a polynucleotide comprising a nucleotide sequence encoding a light chain of any one of claims 99-107, 109-117 and 130-136, a polynucleotide comprising a nucleotide sequence encoding an Fc polypeptide of any one of claims 99-107, 109-117 and 130-136, and a polynucleotide comprising a nucleotide sequence encoding a heavy chain (e.g., a heavy chain comprising a Fc polypeptide capable of binding to a TfR; or a heavy chain linked to a second binding region) of any one of claims 99-107, 109-117 and 130-136.

144. A vector comprising the polynucleotide of claim 141 or 142 or the plurality of polynucleotides of claim 143.

145. One or more vectors comprising the plurality of polynucleotides of claim 143.

146. A host cell comprising the polynucleotide or plurality of polynucleotides of any one of claims 141-143 or a vector(s) of claim 144 or 145.

147. A method of expressing a molecule that specifically binds to TREM2, comprising: culturing the host cell of claim 146 under conditions suitable for expression of the molecule.

148. A method of enhancing the activity of a monovalent anti-TREM2 molecule, the method comprising modifying the molecule to specifically bind to a transferrin receptor.

149. A molecule as described in any one of claims 1-136 or a pharmaceutical composition as described in claim 137 for use in treating a neurodegenerative disease in a subject.

150. The use of a molecule as described in any one of claims 1-136 in the preparation of a medicament for treating a neurodegenerative disease in a subject.

151. A molecule as described in any one of claims 1-136 or a pharmaceutical composition as described in claim 137 for use in enhancing TREM2 activity in a subject having a neurodegenerative disease.

152. The use of a molecule as described in any one of claims 1-136 in the preparation of a medicament for enhancing TREM2 activity in a subject having a neurodegenerative disease.
